SEQUENCE LISTING (89 sequences)

SEQ ID NO: 1 - DNA, 129 bp, Artificial Sequence
  Oligonucleotide cassette for regulation of transcription.
tcgggaaagatctaagttagtgtattgacatgatagaagcactctactatattcctaggagatgctgcatataagcagctgctggtaccaagttcacgttaaaggaaacagaccatgacgcgtattacg

SEQ ID NO: 2 - DNA, 249 bp, Artificial Sequence
  Coding sequence for expression of cro protein of bacteriophage 434
aagcttataaattaaggaggttgtatgcaaactcttagcgaacgcctgaaaaaacgtcgcattgctcttaagatgacgcaaaccgagctcgcaaccaaagccggcgttaaacagcaaagcattcaactgattgaagccggggtaaccaaacgcccgcgcttcctgtttgaaattgctatggcgctgaactgtgatccggtttggctgcagtacggtactaaacgcggtaaagccgcttaataagaattc

SEQ ID NO: 3 - protein, 71 aa, Artificial Sequence
  Synthetic Construct
MQTLSERLKKRRIALKMTQTELATKAGVKQQSIQLIEAGVTKRPRFLFEIAMALNCDPVWLQYGTKRGKAA

SEQ ID NO: 4 - DNA, 3529 bp, Artificial Sequence
  Plasmid containing a promoter, HIV-derived target operator and reporter gene
tcgggaaagatctaagttagtgtattgacatgatagaagcactctactatattcctaggagatgctgcatataagcagctgctggtaccaagttcacgttaaaggaaacagaccatgacgcgtattacgtgctgcaggtcgacggatccggggaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgctcttccgcttcctcgctcactgactcgctgcgctcggtcgttcggctgcggcgagcggtatcagctcactcaaaggcggtaatacggttatccacagaatcaggggataacgcaggaaagaacatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatc

SEQ ID NO: 5 - protein, 149 aa, Artificial Sequence
  Synthetic Construct
MTRITCCRSTDPGNSLAVVLQRRDWENPGVTQLNRLAAHPPFASWRNSEEARTDRPSQQLRSLNGEWRSSASSLTDSLRSVVRLRRAVSAHSKAVIRLSTESGDNAGKNMVKTGAKKLSILATFKSKLVKLTQGLAETKNIFSINPLGK

SEQ ID NO: 6 - DNA, 3197 bp, Artificial Sequence
  Plasmid for expression of mutational libraries of 434 cro repressor
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttataaactaaggaggttgtatgcaaactcttagcgaacgcctgaaaaaacgtcgcattgctcttaagatgacgcaaaccgagctcgcaaccaaagccggcgttaaacagcaaagcattcaactgattgaagccggggtaaccaaacgcccgcgcttcctgtttgaaattgctatggcgctgaactgtgatccggtttggctgcagtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 7 - protein, 71 aa, Artificial Sequence; identical to SEQ ID NO: 3
  Synthetic Construct

SEQ ID NO: 8 - DNA, 18 bp, Artificial Sequence
  Cauliflower mosaic virus derived sequence
ataaggaagttcatttca

SEQ ID NO: 9 - DNA, 3660 bp, Artificial Sequence
  Plasmid containing a promoter, cauliflower mosaic virus-derived target operator and reporter gene and ompA-derived separator gene
ctaggataaggaagttcatttcaggtaccaagttcacgttaaaggaaacagaccatgacgcgtaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacgagctcagcgcttggcgtcacccgcagttcggtggctaacatcatcatcatcatcacggcggcgattataaagatgatgatgataaataagcaagttcacgttaaaggaaacagaccatgacgcgtattacgtgctgcaggtcgacggatccggggaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgctcttccgcttcctcgctcactgactcgctgcgctcggtcgttcggctgcggcgagcggtatcagctcactcaaaggcggtaatacggttatccacagaatcaggggataacgcaggaaagaacatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggacttgaaaagcacaaaagccagtctggaaacaggctggcttttttttgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatctcgggaaagatctaagttagtgtattgacatgatagaagcactctactatattc

SEQ ID NO: 10 - protein, 194 aa, Artificial Sequence
  Synthetic Construct
MTRKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNELSAWRHPQFGG

SEQ ID NO: 11 - protein, 149 aa, Artificial Sequence; identical to SEQ ID NO: 5
  Synthetic Construct

SEQ ID NO: 12 - DNA, 57 bp, Artificial Sequence
  oligonucleotide sequence for promoter - top strand
agatctaagttagtgtattgacatgatagaagcactctactatattcctaggtacca

SEQ ID NO: 13 - DNA, 61 bp, Artificial Sequence
  oligonucleotide sequence for promoter - bottom strand
agcttggtacctaggaatatagtagagtgcttctatcatgtcaatacactaacttagatct

SEQ ID NO: 14 - DNA, 50 bp, Artificial Sequence
  Oligonucleotide for nuclear localization sequence - top strand
agcttataaactaaggaggttgtatgcaaccaaaaaagaagagaaaggtc

SEQ ID NO: 15 - DNA, 37 bp, Artificial Sequence
  Oligonucleotide for nuclear localization sequence - top strand
actcttagcgaacgcctgaaaaaacgtcgcattgctc

SEQ ID NO: 16 - DNA, 36 bp, Artificial Sequence
  Oligonucleotide for nuclear localization sequence - bottom strand
ttcttttttggttgcatacaacctccttagtttata

SEQ ID NO: 17 - DNA, 51 bp, Artificial Sequence
  Oligonucleotide for nuclear localization sequence - bottom strand
ttaagagcaatgcgacgttttttcaggcgttcgctaagagtgacctttctc

SEQ ID NO: 18 - DNA, 3096 bp, Artificial Sequence
  Plasmid for expressing cro repressor variants
agcgcccaatacgcaaaccgcctctccccgcgcgttggccgattcattaatgcagagatctaagttagtgtattgacatgatagaagcactctactatattcctaggtaccaagcttataaactaaggaggttgtatgcaaccaaaaaagaagagaaaggtcactcttagcgaacgcctgaaaaaacgtcgcattgctcttaagatgacgcaaaccgagctcgcaaccaaagccggcgttaaacagcaaagcattcaactgattgaagccggggtaaccaaacgcccgcgcttcctgtttgaaattgctatggcgctgaactgtgatccggtttggctgcagtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 19 - protein, 78 aa, Artificial Sequence
  Synthetic Construct
MQPKKKRKVTLSERLKKRRIALKMTQTELATKAGVKQQSIQLIEAGVTKRPRFLFEIAMALNCDPVWLQYGTKRGKAA

SEQ ID NO: 20 - DNA, 27 bp, Artificial Sequence
  mutagenic cassette primer
cgggcgtttggttaccccggcttcaat

SEQ ID NO: 21 - DNA, 23 bp, Artificial Sequence
  mutagenic cassette primer 2
aacgccggctttggttgcgagct

SEQ ID NO: 22 - DNA, 52 bp, Artificial Sequence
  Mutagenic cassette oligonucleotide containing degeneracies for mutations.
cgcaaccaaagccggcgttnnsnnsnnsnnsattcaannsattgaagccggg

SEQ ID NO: 23 - DNA, 3630 bp, Artificial Sequence
  Plasmid derived from pComp containing a hexahisadine separator gene
ctaggataaggaagttcatttcaggtaccaagttcacgttaaaggaaacagaccatgacgcgtaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggtggccatcatcatcatcatcactaaggcggcgattataaagatgatgatgataaataagcaagttcacgttaaaggaaacagaccatgacgcgtattacgtgctgcaggtcgacggatccggggaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgctcttccgcttcctcgctcactgactcgctgcgctcggtcgttcggctgcggcgagcggtatcagctcactcaaaggcggtaatacggttatccacagaatcaggggataacgcaggaaagaacatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggacttgaaaagcacaaaagccagtctggaaacaggctggcttttttttgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatctcgggaaagatctaagttagtgtattgacatgatagaagcactctactatattc

SEQ ID NO: 24 - protein, 190 aa, Artificial Sequence
  Synthetic Construct
MTRKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGGHHHHHH

SEQ ID NO: 25 - protein, 149 aa, Artificial Sequence; identical to SEQ ID NO: 5
  Synthetic Construct

SEQ ID NO: 26 - DNA, 3603 bp, Artificial Sequence
  Plasmid derived from pComp containing a flag tag separator gene
ctaggataaggaagttcatttcaggtaccaagttcacgttaaaggaaacagaccatgacgcgtaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcggcgattataaagatgatgatgataaataagcaagttcacgttaaaggaaacagaccatgacgcgtattacgtgctgcaggtcgacggatccggggaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgctcttccgcttcctcgctcactgactcgctgcgctcggtcgttcggctgcggcgagcggtatcagctcactcaaaggcggtaatacggttatccacagaatcaggggataacgcaggaaagaacatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggacttgaaaagcacaaaagccagtctggaaacaggctggcttttttttgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatctcgggaaagatctaagttagtgtattgacatgatagaagcactctactatattc

SEQ ID NO: 27 - protein, 192 aa, Artificial Sequence
  Synthetic Construct
MTRKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGGDYKDDDDK

SEQ ID NO: 28 - protein, 149 aa, Artificial Sequence; identical to SEQ ID NO: 5
  Synthetic Construct

SEQ ID NO: 29 - DNA, 360 bp, Artificial Sequence
  repression domain from AtMYB4 encoding amino acid residues 163-282
tcaatggtcgtctcatcgcaacaaggtccatggtggttcccggcgaatacaactacgactaatcaaaactctgcgttttgctttagttcaagtaatactacaacggtttcagaccagatcgtatctttaatctcttcaatgtctacgtcatcatctccgacaccaatgacttcaaacttcagtcctgctccaaacaactgggaacaactcaactactgcaacacagtaccaagtcagagcaacagtatcttcagtgccttctttggtaatcaatacacagaagctagccaaaccatgaacaataataatccactagtagatcaacatcatcatcatcaagacatgaagtcatgggcatca

SEQ ID NO: 30 - protein, 120 aa, Artificial Sequence
  Synthetic Construct
SMVVSSQQGPWWFPANTTTTNQNSAFCFSSSNTTTVSDQIVSLISSMSTSSSPTPMTSNFSPAPNNWEQLNYCNTVPSQSNSIFSAFFGNQYTEASQTMNNNNPLVDQHHHHQDMKSWAS

SEQ ID NO: 31 - DNA, 465 bp, Artificial Sequence
  repression domain from Oshox1 gene encoding amino acid residues 1-155
atggagatgatggttcatgggaggagagacgagcagtatggcgggctcgggctcgggcttgggcttgggctcagcctcggcgtcgccggtggtgcagccgacgacgagcagccgccgccgcgccgtggtgccgccccgccgccgcagcagcagctgtgcggctggaacggcggcggtctcttctcctcgtcttcctccgatcatcgggggaggtcggcgatgatggcgtgccacgacgtcatcgagatgccgttcctacgggggatcgacgtgaaccgtgcgccggcggcagagacgaccacgacgacggcgagggggcccagctgcagcgaggaagacgaggagcccggcgcgtcctcccccaacagcacgctctccagcctcagcggcaagcgcggcgcaccatctgccgccaccgccgccgccgccgccgccagcgacgacgaggactccggcggcggatcc

SEQ ID NO: 32 - protein, 155 aa, Artificial Sequence
  Synthetic Construct
MEMMVHGRRDEQYGGLGLGLGLGLSLGVAGGAADDEQPPPRRGAAPPPQQQLCGWNGGGLFSSSSSDHRGRSAMMACHDVIEMPFLRGIDVNRAPAAETTTTTARGPSCSEEDEEPGASSPNSTLSSLSGKRGAPSAATAAAAAASDDEDSGGGS

SEQ ID NO: 33 - DNA, 330 bp, Artificial Sequence
  Activation domain from GBF-1 protein
atgggaacgagcgaagacaagatgccatttaagactaccaaaccaacatcttcggctcaggaagttcctcccacaccgtatccagattggcaaaattcaatgcaggcttattatggcggaggaggatctccaaatccttttttcccatccccagttggatctcctagtcctcacccctatatgtggggtgctcaacaccatatgatgccgccttatggcaccccagttccgtacccagcaatgtatcccccgggggcagtctatgctcatcctagcatgcccatgcctcctaattctggtcctaccaacaaggagcctgcgaaggaccaa

SEQ ID NO: 34 - protein, 110 aa, Artificial Sequence
  Synthetic Construct
MGTSEDKMPFKTTKPTSSAQEVPPTPYPDWQNSMQAYYGGGGSPNPFFPSPVGSPSPHPYMWGAQHHMMPPYGTPVPYPAMYPPGAVYAHPSMPMPPNSGPTNKEPAKDQ

SEQ ID NO: 35 - DNA, 153 bp, Artificial Sequence
  Opaque2 activation domain
atcgtcgtcggcagtgtcatagacgttgctgctgctggtcatggtgacggggacatgatggatcagcagcacgccacagagtggacctttgagaggttactagaagaggaggctctgacgacaagcacaccgccgccggtggtggtggtgccg

SEQ ID NO: 36 - protein, 51 aa, Artificial Sequence
  Synthetic Construct
IVVGSVIDVAAAGHGDGDMMDQQHATEWTFERLLEEEALTTSTPPPVVVVP

SEQ ID NO: 37 - DNA, 225 bp, Artificial Sequence
  Activation genome
accgatgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatatggccgacttcgagtttgagcagatgtttaccgatgcccttggaattgacgagtacggtggg

SEQ ID NO: 38 - protein, 75 aa, Artificial Sequence
  Synthetic Construct
TDVSLGDELHLDGEDVAMAHADALDDFDLDMLGDGDSPGPGFTPHDSAPYGALDMADFEFEQMFTDALGIDEYGG

SEQ ID NO: 39 - DNA, 333 bp, Artificial Sequence
  activation domain from GAL4 protein
cgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaaccc

SEQ ID NO: 40 - protein, 110 aa, Artificial Sequence
  Synthetic Construct
ANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPN

SEQ ID NO: 41 - DNA, 5996 bp, Artificial Sequence
  Reporter plasmid containing promoter, bacteriophage-derived target operator and nearly full-length LacZ reporter gene
tcgggaaagatctaagttagtgtattgacatgatagaagcactctactatattcctaggaacagtttttcttgtggtaccaagttcacgttaaaggaaacagaccatgacgcgtattacgtgctgcaggtcgacggatccggggaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataagaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatc

SEQ ID NO: 42 - protein, 1032 aa, Artificial Sequence
  Synthetic Construct
MTRITCCRSTDPGNSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 43 - DNA, 3828 bp, Artificial Sequence
  Plasmid for expression of vnk/NK-2 homeodomain DNA binding domain
agcttataaactaaggaggtcatatgtccgacggtctgccaaataagaaacggaagcgacgagtcctgttcaccaaggcgcaaacatatgagctggaacgtcggtttcgacaacaacgttacttgagtgccccggaacgcgagcacctggccagtttgatccgcctgacgccgacccaggtgaagatctggtttcaaaaccatcgctacaagacgaagcgggcgcaaaacgagaagggctacgagggtcatccttaaggatctcggcgtatatcaaagcgcgatcaacaaggccattcatgccggccgaaagatttttttaactataaacgctgatggaagcgtttatgcggaagaggtaaagcccttcccgagtaacaaaaaaacaacagcataaataaccccgctcttacacattccagccctgaaaaagggcatcaaattaaaccacacctatggtgtatgcatttatttgcatacattcaatcaattgttatctaaggaaatacttacatatggttcgtgcaaacaaacgcaacgaggctctacgaatcgagagtgcgttgcttaacaaaatcgcaatgcttggaactgagaagacagcggaagctgtgggcgttgataagtcgcagatcagcaggtggaagagggactggattccaaagttctcaatgctgcttgctgttcttgaatggggggtcgttgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcca

SEQ ID NO: 44 - protein, 77 aa, Artificial Sequence
  Synthetic Construct
MSDGLPNKKRKRRVLFTKAQTYELERRFRQQRYLSAPEREHLASLIRLTPTQVKIWFQNHRYKTKRAQNEKGYEGHP

SEQ ID NO: 45 - DNA, 3195 bp, Artificial Sequence
  Plasmid for expression of ATHB-1 HDLZ-fusion proteins
catgggccagctgccggaaaaaaaacgtcgtctgaccaccgaacaggtgcatctgctggaaaagagcttcgaaaccgaaaacaaactggaaccggaacgtaaaacccagctggcgaaaaaactgggcctgcaaccgcggcaggtggcggtgtggttccagaaccgtcgtgcgcgttggaaaaccaaacagctggaacgtgattatgatctgctgaaaagcacgtacgatcagctgctgagcaactatgatagcattgtgatggataacgataaactgcgtagcgaagtgaccagcctgtaactgcagtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagatctaagttagtgtattgacatgatagaagcactctactatattcctaggtaccaagcttataaactaaggaggttc

SEQ ID NO: 46 - protein, 97 aa, Artificial Sequence
  Synthetic Construct
QLPEKKRRLTTEQVHLLEKSFETENKLEPERKTQLAKKLGLQPRQVAVWFQNRRARWKTKQLERDYDLLKSTYDQLLSNYDSIVMDNDKLRSEVTSL

SEQ ID NO: 47 - DNA, 3287 bp, Artificial Sequence
  Plasmid for expression of zinc finger domains
catgggccatgaacgtccgtacgcgtgcccggtggaaagctgcgatcgtcgtttcagccgtagcgatgaactgacccgtcatattcgtatacataccggccagaaaccgttccagtgccgtatttgcatgcgtaacttcagccgtagcgatcatctgaccacccatattcgtacccataccggcgaaaaaccgttcgcgtgcgatatttgcggccgtaaattcgcgcgtagcgatgaacgtaaacgtcataccaaaattcatctgcgttaactgcagtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttataaactaaggaggttc

SEQ ID NO: 48 - protein, 89 aa, Artificial Sequence
  Synthetic Construct
MGHERPYACPVESCDRRFSRSDELTRHIRIHTGQKPFQCRICMRNFSRSDHLTTHIRTHTGEKPFACDICGRKFARSDERKRHTKIHLR

SEQ ID NO: 49 - DNA, 3189 bp, Artificial Sequence
  Plasmid for expression of zinc finger homeodomain fusion domains
catgggccatgaacgtccgtacgcgtgcccggtggaaagctgcgatcgtcgtttcagccgtagcgatgaactgacccgtcatattcgtatacataccggcggcggccgtaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacccataactgcagtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagatctaagttagtgtattgacatgatagaagcactctactatattcctaggtaccaagcttataaactaaggaggttc

SEQ ID NO: 50 - protein, 97 aa, Artificial Sequence
  Synthetic Construct
MGHERPYACPVESCDRRFSRSDELTRHIRIHTGGGRRRKKRTSIETNIRVALEKSFLENQKPTSEEITMIADQLNMEKEVIRVWFCNRRQKEKRINP

SEQ ID NO: 51 - DNA, 3285 bp, Artificial Sequence
  Plasmid for expression of zinc finger leucine zipper domain fusion
gtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagatctaagttagtgtattgacatgatagaagcactctactatattcctaggtaccaagcttataaactaaggaggttccatgggccatgaacgtccgtacgcgtgcccggtggaaagctgcgatcgtcgtttcagccgtagcgatgaactgacccgtcatattcgtatacataccggccagaaaccgttccagtgccgtatttgcatgcgtaacttcagccgtagcgatcatctgaccacccatattcgtacccataccggcgaaaaaccgttcgcgtgcgatatttgcggccgtaaattcgcgcgtagcgatgaacgtaaacgtcataccaaaattcatctgcgtggcggcggccagctggaacgtgattatgatctgctgaaaagcacgtacgatcagctgctgagcaactatgatagcattgtgatggataacgataaactgcgtagcgaagtgaccagcctgtaactgca

SEQ ID NO: 52 - protein, 129 aa, Artificial Sequence
  Synthetic Construct
MGHERPYACPVESCDRRFSRSDELTRHIRIHTGQKPFQCRICMRNFSRSDHLTTHIRTHTGEKPFACDICGRKFARSDERKRHTKIHLRGGGQLERDYDLLKSTYDQLLSNYDSIVMDNDKLRSEVTSL

SEQ ID NO: 53 - DNA, 3294 bp, Artificial Sequence
  Plasmid for expression of zinc finger homeodomain leucine zipper domains
catgggccatgaacgtccgtacgcgtgcccggtggaaagctgcgatcgtcgtttcagccgtagcgatgaactgacccgtcatattcgtatacataccggcggcggccagctgccggaaaaaaaacgtcgtctgaccaccgaacaggtgcatctgctggaaaagagcttcgaaaccgaaaacaaactggaaccggaacgtaaaacccagctggcgaaaaaactgggcctgcaaccgcggcaggtggcggtgtggttccagaaccgtcgtgcgcgttggaaaaccaaacagctggaacgtgattatgatctgctgaaaagcacgtacgatcagctgctgagcaactatgatagcattgtgatggataacgataaactgcgtagcgaagtgaccagcctgtaactgcagtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagatctaagttagtgtattgacatgatagaagcactctactatattcctaggtaccaagcttataaactaaggaggttc

SEQ ID NO: 54 - protein, 132 aa, Artificial Sequence
  Synthetic Construct
MGHERPYACPVESCDRRFSRSDELTRHIRIHTGGGQLPEKKRRLTTEQVHLLEKSFETENKLEPERKTQLAKKLGLQPRQVAVWFQNRRARWKTKQLERDYDLLKSTYDQLLSNYDSIVMDNDKLRSEVTSL

SEQ ID NO: 55 - DNA, 4044 bp, Artificial Sequence
  Plasmid for expression of zinc finger progesterone-dependent dimerization domain fusions
gtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagatctaagttagtgtattgacatgatagaagcactctactatattcctaggtaccaagcttataaactaaggaggttccatgggccatgaacgtccgtacgcgtgcccggtggaaagctgcgatcgtcgtttcagccgtagcgatgaactgacccgtcatattcgtatacataccggccagaaaccgttccagtgccgtatttgcatgcgtaacttcagccgtagcgatcatctgaccacccatattcgtacccataccggcgaaaaaccgttcgcgtgcgatatttgcggccgtaaattcgcgcgtagcgatgaacgtaaacgtcataccaaaattcatctgcgtggcggcggcgtcagagttgtgagagcactggatgctgttgctctcccacagccattgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacatgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacagcttcatctgtactgcttgaatacatttatccagtcccgggcactgagtgttgaatttccagaaatgatgtctgaagttattgctgcacaattacccaagatattggcagggatggtgaaaccccttctctttcataaaaagtgataactgca

SEQ ID NO: 56 - protein, 381 aa, Artificial Sequence
  Synthetic Construct
MGHERPYACPVESCDRRFSRSDELTRHIRIHTGQKPFQCRICMRNFSRSDHLTTHIRTHTGEKPFACDICGRKFARSDERKRHTKIHLRGGGVRVVRALDAVALPQPLGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 57 - DNA, 4212 bp, Artificial Sequence
  Plasmid for expression of zinc finger estrogen receptor dimerization domain fusions
gtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagatctaagttagtgtattgacatgatagaagcactctactatattcctaggtaccaagcttataaactaaggaggttccatgggccatgaacgtccgtacgcgtgcccggtggaaagctgcgatcgtcgtttcagccgtagcgatgaactgacccgtcatattcgtatacataccggccagaaaccgttccagtgccgtatttgcatgcgtaacttcagccgtagcgatcatctgaccacccatattcgtacccataccggcgaaaaaccgttcgcgtgcgatatttgcggccgtaaattcgcgcgtagcgatgaacgtaaacgtcataccaaaattcatctgcgtggcggcggcatgaaaggcggcatacggaaagaccgccgaggagggagaatgttgaagcacaagcgtcagagagatgacttggaaggccgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgcttgctacgtcaagtcggttccgcatgatgaacctgcagggtgaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaaggcatggagcatctctacaacatgaaatgcaagaacgttgtgcccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctgataactgca

SEQ ID NO: 58 - protein, 437 aa, Artificial Sequence
  Synthetic Construct
MGHERPYACPVESCDRRFSRSDELTRHIRIHTGQKPFQCRICMRNFSRSDHLTTHIRTHTGEKPFACDICGRKFARSDERKRHTKIHLRGGGMKGGIRKDRRGGRMLKHKRQRDDLEGRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI

SEQ ID NO: 59 - DNA, 4439 bp, Artificial Sequence
  Plasmid for bacterial transcriptional activation screening
catgggccctcaacagcagcaaatgcaacctcccaattcaagtgcgaacaacaaccctttgcaacagcaatcatcacaaaataccgtaccaaacgtcctcaaccaaattaaccaaatcttttctcaagaggagcaacgcagcttattacaagaagccatcgaaacctgcaagaattttgaaaaaacacaattgggtagtacgatgacggaacctgtcaagcaaagttttattaggaaatacattaaccaaaaggccctgagaaaaatccaagctttggcggcggcgccgcgtgtgcgtaccggcagcaagacacccccccatgaacgtccgtacgcgtgcccggtggaaagctgcgatcgtcgtttcagccgtagcgatgaactgacccgtcatattcgtatacataccggccagaaaccgttccagtgccgtatttgcatgcgtaacttcagccgtagcgatcatctgaccacccatattcgtacccataccggcgaaaaaccgttcgcgtgcgatatttgcggccgtaaattcgcgcgtagcgatgaacgtaaacgtcataccaaaattcatctgcgtcagaaggactaagctagcttataaactaaggaggttccatgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgccgccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagaggcggcggcggtggaaagccgtctggaacgtctggaacagctgttcctgctgattttcccgcgtgaagatctggatatgattctgaaaatggatagcctgcaagatattaaagcgctgctgaccggcctgttctaaacttgaaaagcacaaaagccagtctggaaacaggctggcttttttttgctgcagtacggtactaaacgcggtaaagccgcttaataagaattgcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagatctaagttagtgtattgacatgatagaagcactctactatattcctaggtaccaagcttataaactaaggaggttc

SEQ ID NO: 60 - protein, 196 aa, Artificial Sequence
  Synthetic Construct
MGPQQQQMQPPNSSANNNPLQQQSSQNTVPNVLNQINQIFSQEEQRSLLQEAIETCKNFEKTQLGSTMTEPVKQSFIRKYINQKALRKIQALAAAPRVRTGSKTPPHERPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 61 - protein, 292 aa, Artificial Sequence
  Synthetic Construct
MQGSVTEFLKPRLVDIEQVSSTHAKVTLEPLERGFGHTLGNALRRILLSSMPGCAVTEVEIDGVLHEYSTKEGVQEDILEILLNLKGLAVRVQGKDEVILTLNKSGIGPVTAADITHDGDVEIVKPQHVICHLTDENASISMRIKVQRGRGYVPASTRIHSEEDERPIGRLLVDACYSPVERIAYNVEAARVEQRTDLDKLVIEMETNGTIDPEEAIRRAATILAEQLEAFVDLRDVRQPEVKEEKPEAAAVESRLERLEQLFLLIFPREDLDMILKMDSLQDIKALLTGLF

SEQ ID NO: 62 - DNA, 4543 bp, Artificial Sequence
  Plasmid 2 for bacterial transcriptional activation screening
ctagcgatctagcgtgggcgcacctaggagctcctaggagctcctaggagctcctaggagctcctaggaagttagtgtattgacatgatagaagcactctactatatcctggtaccttataaactaaggaggttccatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggccagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgaacttgaaaagcacaaaagccagtctggaaacaggctggcttttttttgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatctcgggaaagatctagcgtgggcgcacctaggagctcctaggagctcctaggagctcctaggagctcctaggaagttagtgtattgacatgatagaagcactctactatatcctggtaccaagttcacgttaaaggaaacagaccatgacgcgtaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacgagctcagcgcttggcgtcacccgcagttcggtggctaacatcatcatcatcatcacggcggcgattataaagatgatgatgataaataagcaagttcacgttaaaggaaacagaccatgacgcgtattacgtgctgcaggtcgacggatccggggaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgctcttccgcttcctcgctcactgactcgctgcgctcggtcgttcggctgcggcgagcggtatcagctcactcaaaggcggtaatacggttatccacagaatcaggggataacgcaggaaagaacatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggacttgaaaagcacaaaagccagtctggaaacaggctggcttttttttg

SEQ ID NO: 63 - protein, 219 aa, Artificial Sequence
  Synthetic Construct
MTEQKALVKRITNETKIQIAISLKGGPLAIEHSIFPEKEAEAVAEQATQSQVINVHTGIGFLDHMIHALAKHSGWSLIVECIGDLHIDDHHTTEDCGIALGQAFKEALLARGVKRFGSGFAPLDEALSRAVVDLSNRPYAVVELGLQREKVGDLSCEMIPHFLESFAEASRITLHVDCLRGKNDHHRSESAFKALAVAIREATSPNGTNDVPSTKGVLM

SEQ ID NO: 64 - protein, 194 aa, Artificial Sequence; identical to SEQ ID NO: 10
  Synthetic Construct

SEQ ID NO: 65 - protein, 149 aa, Artificial Sequence; identical to SEQ ID NO: 5
  Synthetic Construct

SEQ ID NO: 66 - DNA, 1386 bp, Artificial Sequence
  Insert for construction of M2BA1cro1 bacteriophage vector
gaccgggcccacatagatctaagttagtgtattgacatgatagaagcactctactatattcctaggaaaaatatgtgttttggtaccaagttcacgttaaaggaaacagaccatgaaaaagtctttagtcctcaaagcctctgtagccgttgctaccctcgttccgatgctaagctttgcccatcaccatcaccaccatcctgcagaaggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaataagcaagttcacgttaaaggaaacagaccatgaccatgattacggatccggggaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgctcttccgcttcctcgctcactgactcgctgcgctcggtcgttcggctgcggcgagcggtatcagctcactcaaaggcggtaatacggttatccacagaatcaggggataacgcaggaaagaacatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggacttgaaaagcacaaaagccagtctggaaacaggctggcttttttttgctagcaattgagatctaagttagtgtattgacatgatagaagcactctactatattcctaggtaccaagcttataaactaaggaggttgtatgcaaactcttagcgaacgcctgaaaaaacgtcgcattgctcttaagatgacgcaaaccgagctcgcaaccaaagccggcgttaaacagcaaagcattcaactgattgaagccggggtaaccaaacgcccgcgcttcctgtttgaaattgctatggcgctgaactgtgatccggtttggctgcagtacggtactaaacgcggtaaagccgcttaataagaattcgcatgcactagccc

SEQ ID NO: 67 - protein, 80 aa, Artificial Sequence
  Synthetic Construct
MKKSLVLKASVAVATLVPMLSFAHHHHHHPAEGDDPAKAAFNSLQASATEYIGYAWAMVVVIVGATIGIKLFKKFTSKAS

SEQ ID NO: 68 - protein, 144 aa, Artificial Sequence
  Synthetic Construct
MTMITDPGNSLAVVLQRRDWENPGVTQLNRLAAHPPFASWRNSEEARTDRPSQQLRSLNGEWRSSASSLTDSLRSVVRLRRAVSAHSKAVIRLSTESGDNAGKNMVKTGAKKLSILATFKSKLVKLTQGLAETKNIFSINPLGK

SEQ ID NO: 69 - protein, 71 aa, Artificial Sequence; identical to SEQ ID NO: 3
  Synthetic Construct

SEQ ID NO: 70 - DNA, 109 bp, Artificial Sequence
  upstream transcription terminator variant for M2BA1cro1
gaccgggcccacatacttgaaaagcacaaaagccagtctggaaacaggctggcttttttttgagatctaagttagtgtattgacatgatagaagcactctactatattc

SEQ ID NO: 71 - DNA, 3446 bp, Artificial Sequence
  Phagemid vector for combinatorial libraries of binding proteins
ctagcaattgagatctaagttagtgtattgacatgatagaagcactctactatattcctaggtaccaagcttataaactaaggaggttgtatgcaaactcttagcgaacgcctgaaaaaacgtcgcattgctcttaagatgacgcaaaccgagctcgcaaccaaagccggcgttaaacagcaaagcattcaactgattgaagccggggtaaccaaacgcccgcgcttcctgtttgaaattgctatggcgctgaactgtgatccggtttggctgcagtacggtactaaacgcggtaaagccgcttaataagaattcgcatgcactagccctgaggccgatacggtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaacgcgaattttaacaaaatattaacgtttacaatttctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 72 - protein, 71 aa, Artificial Sequence; identical to SEQ ID NO: 3
  Synthetic Construct

SEQ ID NO: 73 - protein, 286 aa, Artificial Sequence
  Synthetic Construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 74 - DNA, 14 bp, Artificial Sequence
  Target binding sequence 1 for Bacillus anthracis atxA
aaaatatgtgtttt

SEQ ID NO: 75 - DNA, 14 bp, Artificial Sequence
  Target binding sequence 2 for Bacillus anthracis atxA
atgtgttttgaaat

SEQ ID NO: 76 - DNA, 14 bp, Artificial Sequence
  Target binding sequence 3 for Bacillus anthracis atxA
aatataatagcatt

SEQ ID NO: 77 - DNA, 14 bp, Artificial Sequence
  Target binding sequence 4 for Bacillus anthracis atxA
atataatagcattt

SEQ ID NO: 78 - DNA, 14 bp, Artificial Sequence
  Target binding sequence 5 for Bacillus anthracis atxA
ataatagcatttgt

SEQ ID NO: 79 - DNA, 14 bp, Artificial Sequence
  Target binding sequence 1 for Bacillus anthracis pagA
aatataaatttaat

SEQ ID NO: 80 - DNA, 14 bp, Artificial Sequence
  Target binding sequence 2 for Bacillus anthracis pagA
ataaatttaatttt

SEQ ID NO: 81 - DNA, 14 bp, Artificial Sequence
  Target binding sequence 3 for Bacillus anthracis pagA
taaatttaatttta

SEQ ID NO: 82 - DNA, 14 bp, Artificial Sequence
  Target binding sequence 4 for Bacillus anthracis pagA
tttaattttataca

SEQ ID NO: 83 - DNA, 13 bp, Artificial Sequence
  Target binding sequence 1 for variola virus
atacccataaatg

SEQ ID NO: 84 - DNA, 13 bp, Artificial Sequence
  Target binding sequence 2 for variola virus
agctatttaaatg

SEQ ID NO: 85 - DNA, 13 bp, Artificial Sequence
  Target binding sequence 3 for variola virus
acatatctaaatg

SEQ ID NO: 86 - DNA, 13 bp, Artificial Sequence
  Target binding sequence 4 for variola virus
aaaatttaaaatg

SEQ ID NO: 87 - DNA, 13 bp, Artificial Sequence
  Target binding sequence 5 for variola virus
tgtacaataaatg

SEQ ID NO: 88 - DNA, 13 bp, Artificial Sequence
  Target binding sequence 6 for variola virus
tcactcataaatg

SEQ ID NO: 89 - DNA, 13 bp, Artificial Sequence
  Target binding sequence 7 for variola virus
atttgatataatg

(19) United States
(12) Patent Application Publication
(10) Pub. No.: US 2004/0161753 A1
(43) Pub. Date: Aug. 19, 2004

(54) Creation and identification of proteins having new dna binding specificities

(76) Inventors: John G. Wise, Allen, TX (US); Katja Fromknecht, Ebersberg (DE)

Correspondence Address: HELLER EHRMAN WHITE & MCAULIFFE LLP, 1666 K STREET,NW, SUITE 300, WASHINGTON, DC, 20006, US

(21) Appl. No.: 10/416,708

(22) PCT Filed: Nov. 16, 2001
(86) PCT No.: PCT/US01/43107

(51) Int. Cl. C12Q 1/68; C12N 15/85

(52) U.S. Cl. 435/6; 435/455

(57) ABSTRACT

Methods are provided for identification and production of new DNA binding proteins that up or down regulate the expression of pre-determined target genes. Such genes include DNA sequences that encode proteins that regulate such target genes as well as gene constructs and biological materials that contain such DNA binding proteins and/or their DNA sequences. Discovery methods also are provided for transcriptional promoters that allow identification of the desired target gene specific DNA binding proteins, methods for targeting DNA binding protein variants to the desired DNA binding sequence, the methods for removing undesired DNA binding protein variants from the total pool of all variants, as well as the media used for assaying in vivo DNA binding. The invention further encompasses kits for the identification and production of DNA binding protein variants and/or their DNA sequences.

### FIELD OF THE INVENTION

The invention relates to DNA binding proteins and methods of creating new regulatory proteins.

### BACKGROUND OF THE INVENTION

DNA binding proteins regulate the activity of genes or set of genes through their effects on transcription. The regulation typically occurs through binding to DNA. Accordingly, the term “DNA binding proteins” has been adopted to mean the large class of proteins that bind and regulate DNA. Features of this binding may be understood through the specific three-dimensional structure of the protein and of the DNA, which provides information of interactions between the protein and the nucleotide bases and/or sugar-phosphate-backbone moieties of the DNA.

DNA binding proteins naturally occur. Jacob and Monod proposed the operon model as a model of simple gene regulation in 1961. This regulatory system encompasses gene-regulated transcription (and thereby gene activity regulation). The regulatory system comprises, as a minimum, a regulatory gene that encodes a DNA binding protein that influences DNA transcription, a promoter where RNA synthesis is initiated, an operator that consists of at least one transcriptional control sequence and a structural gene (protein-coding gene) that can be regulated.

Work subsequent to elucidation of the early operon model has shown that regulator substances, repressors and/or activators of gene transcription often control gene expression. The regulatory mechanisms of transcription and gene expression differ between prokaryotic and eukaryotic organisms. However, the basis for the regulation is similar. Protein regulators of gene transcription bind to specific DNA sequences and inhibit or activate the transcription of one or more genes. More than one regulator protein (as an activator or inhibitor) often binds a given gene or gene set through binding to one or more DNA sequences. When present in a prokaryote the DNA binding site often is termed an “operator.” When present in a eukaryote the DNA binding sequence often is termed an “activator,” “activator sequence,” “enhancer,” or “enhancer sequence.” Additional DNA sequences are important for transcription and transcriptional regulation. These further sequences form binding sites for general transcription factors such as proteins used for gene transcription generally. One such transcription factor is an RNA polymerase. A DNA sequence that binds an RNA polymerase generally is termed a “promoter” and is important for transcription control.

Mutation of DNA Binding Proteins

Attempts have been made to modify the DNA binding properties of proteins that affect gene regulation by their interactions with regulatory sequences. For example, the ability of mutant 434 repressor and Tet repressor proteins to bind to operator sequences has been analyzed and specificity changes for a few mutants have been reported (Huang et al, 1994; Baumeister et al, 1992). The success of these methods and others at altering the binding specificities appears limited to quite modest changes (Bass et al, 1988; Lehming et al., 1988; 1990; Backes et al, 1997; Huang et al, 1994). One problem with these approaches has often been the low stability of the resultant proteins (Backes et al, 1997; Kalkof et al, 1992). Random mutagenesis of the first two residues of the 434 repressor recognition helix did not lead to the identification of any cro variants with new and specific DNA binding properties (Wharton and Ptashne, 1987).

Engineering DNA Binding Protein Specificity Changes

Typically, four techniques and combinations of them have been used to re-engineer DNA binding proteins to alter the specific binding of the proteins to new DNA sequences. One technique is the so called “rational redesign” method wherein a new protein is engineered from a known DNA binding protein having at least some specificity for a desired DNA sequence. A second technique herein termed “reporter screening systems,” evolves new DNA binding proteins in vitro through phenotype screening of mutations or mutational libraries of DNA binding proteins. A third technique termed “physical separation systems” physically selects protein variants through the extracellular display of the DNA binding domains of those variants. The fourth technique uses in vivo genetic selection of mutant DNA binding proteins that repress or transactivate one or more gene that inhibit growth or survival. Each of these techniques and their combinations have problems, as briefly summarized below.

Rational Redesign of DNA Binding Specificities

The goal of rationally redesigning a given protein such that a specific variant exhibits a desired DNA binding specificity is, in light of the complexity of protein DNA interactions even in the simplest of proteins, one that is achieved only in very special, limited circumstances. One approach to this redesign has been demonstrated in several “helix swapping” experiments. In these experiments, the amino acid residue sequences of the recognition helices of different HTH proteins have been genetically exchanged and the effects on DNA binding studied (Brent and Ptashne, 1985; Kohlkof et al., 1992; Wharton et al., 1984; Backes et al., 1997; Bushman and Ptashne, 1988). Wharton et al (1984) changed the amino acid sequence of the recognition helix of the 434 repressor protein to that of the 434 cro protein and reported the conversion of binding specificity of the mutated repressor to that of the cro protein. Similarly, Wharton and Ptashne (1985) substituted the recognition helix amino acid sequence of the 434 repressor with that of the P22 repressor protein and reported the conversion of binding specificity to that of the P22 protein.

In other helix-swap experiments between 434 repressor and λ repressor, or λ cro and CAP, the hybrid proteins lost functionality (Wharton, 1985). Hollis et al (1988) demonstrated specific binding between a nonpalindromic chimeric operator and a heterodimeric repressor created from wildtype 434 repressor and 434 repressor monomers possessing P22 repressor recognition helix sequences. Similarly, Simoncsits et al. (1999) prepared high affinity variants of a single chain 434 repressor that recognized a nonpalindromic 434/P22 chimeric operator that had a one-base change in its DNA sequence.

Although these studies show individual alterations of DNA binding protein specificity, they do not adequately provide a way to generate large libraries of new proteins that can be screened for variants that bind any new DNA binding sequences. In these helix swapping experiments, for example, one is limited to naturally occurring or known monomeric recognition sequences that can then be incorporated into new variants. The de novo redesign of proteins to bind any desired DNA sequence, i.e. the rational redesign from purely theoretical considerations without reference to experimentation or analysis of natural proteins as in the helix swapping experiments, is a problem that is at present much too complex to be solved generally.

A type of rational redesign technology is described in U.S. Pat. No. 5,554,520, which teaches how to construct gene expression regulator proteins through the use of heterodimeric DNA repressors. In this technique, two different monomers are expressed within the same cell to create a heterodimeric repressor. The monomers possess interacting dimeric interfaces but recognize different DNA binding sequences. This method can generate heterodimeric DNA binding proteins using helix swapping methods that no longer require a palindromic or partially palindromic DNA binding sequence.

Although the methods presented work for the rational combination of monomers with known half-site specificities, this patent fails to show methods whereby desired half-site specificities can be generated. The inventors cite literature that uses genetic selection systems like the phage challenge system reported by Youderian et al. 1983 or the reporter gene system such as that described by Wharton & Ptashne, 1987 to generate such variants. These methods while reporting modest changes in DNA binding have not been successful in creating variants with large DNA binding specificity changes. This lack of success is likely due to the problems discussed more thoroughly below for reporter screening systems, the limited library sizes that are usable with such reporter systems and the resistance problems inherent in genetic selection systems. In contrast, methods are needed that overcome these problems and that can adequately identify DNA binding variants with new and widely different from wildtype binding specificities. Such needed methods would relieve the requirement of a pre-existing and/or known half-site binding specificity.

Reporter Screening Systems

More progress in redesigning binding protein specificities has been made using in vivo phenotypic screening systems. In this approach, systems have been developed that employ mutations or mutational libraries of DNA binding proteins together with the screening of individual clones for a reporter phenotype that is dependent on a protein variant binding to a desired target DNA sequence.

PCT WO97/37030 describes a such a method for selecting seven amino acid long peptides that repress a reporter gene through a zinc-finger motif structure. This method is similar to that reported by Simonscits et al. (1999) where a reporter gene is used to screen for protein variants that function as repressors. This latter work describes the construction of combinatorial libraries of mutations of single chain variants of 434 repressor and the phenotypic screening of the libraries for desired DNA binding specificities. All of these methods suffer from the serious limitation that their use with libraries larger than 104 to 105 members becomes very burdensome, since at least each individual member of the library should be scored for its respective phenotype. In fact in the Simonscits et al. work, the pool of theoretical library members was not completely screened. In addition, the lack of methods for the optimization of the repression process targeted at the desired specific DNA sequences in these methodologies, such as the variation of promoter strength to match repressor strength, leaves little room for the modulation of the phenotypes so that false negative and false positives are not included or excluded from the pool of positively identified variants.

The reporter screening methods are disadvantageous since screening of larger libraries for phenotypic traits is difficult if not impossible. These methods also fail to teach how to balance reporter gene expression through selection methods used on the target gene promoter.

Physical Separation Systems

U.S. Pat. No. 5,789,538 shows a phage display/physical screening method that selects for zinc-finger variants that bind to desired target DNA sequences using a library of DNA sequences. The DNA sequences encode zinc-fingers with mutational variations at presumed and known DNA-protein interfaces. The selected protein variants differ in sequence from wildtype forms and their DNA sequence binding specificities can be selected from a large phage set that displays different zinc-fingers. Further descriptions of this type of technology are found in U.S. Pat. Nos. 6,242,568, 6,013,453, 5,223,409 and 5,571,698.

These in vitro/ex vivo technologies while useful, suffer several disadvantages. Importantly, the conditions in which a protein functions in DNA binding external to the cell as a part of a phage particle are distinctly different from those conditions within the cell where presumably any useful DNA binding protein variant will find its application. These differences can be numerous. Among them are, for example, cooperative interactions with other proteins of the cell including those of the transcriptional machinery, the physical stability of the 3-dimensional structure of the protein variants under in vivo conditions, and the proteolytic sensitivity of the protein variants.

The fact that the DNA binding that is to be selected from phage display experiments occurs externally to the cell and is separated in space from the compartment in which it naturally occurs means that only DNA binding characteristics will be selected and that any other function or characteristic of the DNA binding protein will be ignored by the phage display system. This can lead to the identification of variants that might not reflect the normal mechanism of transcriptional control that operate within the cell and precludes the selection of protein variants that function in cooperation with other DNA binding or transcription-effecting proteins (whether known or unknown) in transactivation and transcriptional repression processes. In addition, instabilities in the protein structure of the variants due to the differences between cell internal and external milieus will not be adequately controlled. An example of this latter effect would be the difference in the intracellular chemical reducing potential relative to that of the extracellular environment. The high reducing potential inside the cell can, for example, reduce and break disulfide bonds that had stabilized a variant structure in the external phage display selection. This can result in identification of DNA binding protein variants not suitable for intracellular applications.

Other problems that might escape detection in the ex vivo/in vitro phage display systems and that would result in proteins not suitable for intracellular in vivo use are instabilities due to the introduction of proteolytic cleavage sites in the variant protein, either directly through a mutagenic change in protein sequence encoded by the library used or indirectly through a lowering of the overall 3-dimensional stability of the protein such that normally hidden proteolytic cleavage sites become dynamically more available for recognition by endogenous, intracellular proteases. Because of differences in the extracellular and intracellular environments in the type and amount of protease activity present, binding variants identified in extracellular selections might be not suitable for intracellular applications. One might identify through such phage display methods poor DNA binding variants that do not function well in internal cellular environments because of such instabilities. Still other problems inherent in the phage display systems have to do with the export of the protein variants through the cell membrane to their desired positions on the surface of the phage. It is not likely that all protein sequences are equally amenable to such export. This may in fact result in under-representation of some sequences and over-representation of other sequences in the library.

In Vivo Selection Systems

U.S. Pat. Nos. 5,096,815 and 5,198,346 describe new DNA binding proteins, in particular repressor proteins, generated through combinatorial mutagenesis of the DNA encoding the proteins, that possess new DNA binding specificities that are identified through genetic selection systems that target DNA binding to desired DNA sequences. The repressor proteins described here are proteins that are similar to normal wildtype proteins except at a number of positions within the gene that encode the protein. Such gene mutational libraries of the DNA binding protein are inserted into a plasmid or other suitable vector for protein expression and are incorporated into a bacterial cell by standard molecular biological techniques. DNA targets of the binding protein variants also may be incorporated into a plasmid. The target sequence functions as a regulatory operator for a structural gene, that when expressed, provides a selective disadvantage to cell growth. When a protein variant binds to its target operator sequence and represses transcription of the deleterious structural gene, the affected cell acquires a selective growth advantage.

Unfortunately, the techniques taught in these patents are limited (for example) by resistances to the disadvantageous gene expression that are generated and expressed by the cell when subjected to the action of the disadvantageous gene. One problem in the repression of transcription of such disadvantageous genes is that repression is seldom if ever complete. Incomplete repression may then exert a selective Darwinian pressure on the culture to eliminate the expression of the disadvantageous gene either by partial or complete elimination of the disadvantageous gene sequences and their activities by deletion or mutation, or, by elimination of the expression of the disadvantageous gene sequences by mutation of promoter and or other control sequences. Second site mutations that generate resistance to the disadvantageous gene are also possible, as are other processes, for example, up-regulation of gene products that interfere with the disadvantageous gene or down-regulation of gene products required for the disadvantageous gene action.

These limitations are particularly noticeable when preparing large library sets having more than 100,000 members, more than 1,000,000 members, more than 10,000,000 members and so on, because the probability of finding such resistances rises as library size becomes larger. This is a serious limitation to the formation and use of libraries for developing new DNA binding proteins.

As with such repressional systems, transcriptional activation systems, such as the bacterial two-hybrid system described by Joung et al. (2000) and other related eukaryotic systems (Wilson et al., 1984; Chien et al., 1991) also may suffer disadvantageous effects from genetic pressure. Joung et al. report, for example, the occurrence of a relatively high rate of background antibiotic resistance that can be found in their system. This serious problem presumably is attributable to undesirable selective pressure that resulted in increased spectinomycin resistance that was not dependent on the desired DNA binding protein transactivation and that results in increased false positive identification when activation of antibiotic resistance was selected. Although such antibiotic resistance breakthrough is relatively obvious to observe in such experiments, it can be expected that similar processes of selective pressure exerted through, for example, the activation of an auxotrophic complementation gene such as the HIS3 gene commonly used in such two-hybrid and one-hybrid systems also contributes significantly to the false positive rates observed with these systems.

Summary of Problems with the Prior Art

The rational design of DNA binding protein specificities is severely limited in the scope of the variations of binding specificity that can be made. Experimentation has shown that many such attempts fail due to unrecognized complexities arising from problems like protein stability and other subtle intricacies of protein structure function relationships. These approaches, while interesting, are not capable of identifying variants with a wide range of different binding specificity changes.

A number of the techniques described above use relatively simple reporter gene transcription systems to report the presence in phenotypic screening experiments of DNA binding protein variants that bind desired target sequences. These techniques do not take into consideration the necessity of balancing the effects of different target DNA sequences on the reporter gene transcriptional activities and thereby not generally applicable to all target sequences. In addition, these methods suffer from the limitation that each individual clone in the library needs to be scored in the screening system for the effect of the DNA binding protein variant on the phenotype of the reporter gene transcription. While useful for relatively small combinatorial libraries of mutations, these systems are not practical for use with larger libraries. Thus, while interesting, these technologies have severe limitations.

In other patents, physical separation techniques that function externally to the cell (phage display, for example) are used to select DNA binding variants that bind desired DNA sequences. While these methods are useful, they suffer from the disadvantage that the selected characteristic, namely DNA binding, is not occurring where it will eventually find its utility. The properties of protein stability, proteolytic susceptibility, protease activities, ability to be exported through a membrane, and the ability to interact with the natural transcriptional regulatory mechanism are different in an extracellular relative to intracellular environment. These differences may result in the identification of DNA binding protein variants that while technically are not false positives, may have limited utility in the desired intracellular environment. Thus the techniques, while also interesting, also have severe limitations.

Several of the techniques described above rely on relief of a negative regulator (whether it be, for example, a toxin, a toxic metabolite, removal of auxotrophic growth regulation, or other characteristic) to select desired regulatory protein variants. In these technologies, cells that contain a DNA protein variant that represses or activates the selection gene by binding the operator target sequence will grow, while those lacking the successful DNA binding protein variant are inhibited. These successful variants are identifiable as cells which escape negative growth selection of the disadvantageous structural gene. Unfortunately, however, strong evolutionary pressure exists in these negative selection systems and which create false positive samples. That is, any mutation that confers partial or complete resistance to the imposed selection will relieve the growth inhibition and contaminate the desired cells that are selected as harboring a desired protein variant that binds the target DNA. It is difficult to separate these contaminating false positives and as libraries become larger the frequency of false positive increases. Thus, while interesting, these technologies also have severe limitations.

The problems with the relevant prior art discussed above can be summarized as follows: The theoretical difficulties, inherent complexity and the lack of complete understanding of DNA-protein interactions severely limit the number of succesful rational and de novo redesign approaches; the lack of balanced gene expression and the “small library” limitations of the reporter-only systems severely limit the breadth of applicability of these approaches; the selected resistance problems that the in vivo selection systems create, leads to missed or false identifications in these systems; and the unnatural, extracellular conditions that do not adequately take into account stability, protease sensitivity, protein export characteristics and complex inter-protein interactions of the intracellular environment limit the physical separation technologies.

### SUMMARY OF THE INVENTION

The problems identified above are alleviated by inventive methods and tools that create new DNA binding proteins that positively and/or negatively regulate the expression of desired gene sequences. New DNA binding gene sequences include DNA sequences that encode proteins that regulate such target genes as well as gene constructs and biological materials that contain such DNA binding proteins and/or their DNA sequences. The invention also encompasses methods for discovering transcriptional promoters. Embodiments of these methods: a) identify desired target genes specific for DNA binding proteins; b) target DNA binding protein variants to desired DNA binding sequences; c) remove undesired DNA binding protein variants from a larger library of variants; and d) provide media useful to assay in vivo DNA binding. The invention further encompasses kits to identify and produce DNA binding protein variants and/or their DNA sequences.

One embodiment of the invention is a method for deriving a gene sequence of a DNA binding protein that can bind to a target regulatory sequence, comprising the steps of selecting a starting DNA sequence for a DNA binding protein, mutating the selected sequence, providing a mutated DNA sequence to a cell that has at least one genetically neutral transcriptional unit wherein the transcriptional unit comprises at least one promotor, at least one reporter gene or separator gene and at least one copy of the target regulatory sequence, wherein binding between the DNA binding protein encoded by the mutated sequence and the target regulatory sequence regulates the expression of the at least one reporter gene or separator gene, and screening for the regulated expression of a gene from the transcriptional unit.

In another embodiment the invention is a method for deriving a gene sequence of a useful DNA binding protein that binds to a target DNA regulatory sequence comprising the steps of selecting a DNA sequence that encodes a protein, mutating the selected sequence, providing a mutated DNA sequence to a cell that has at least one genetically neutral transcriptional unit wherein the transcriptional unit comprises at least one promotor and at least one reporter gene or separator gene and at least one copy of the target DNA regulatory sequence, wherein binding between the DNA binding protein encoded by the mutated sequence and the target regulatory sequence regulates the expression of the at least one reporter gene or separator gene, and screening for expression of a gene by the transcriptional unit.

In other embodiments such determined sequences are used in therapeutics, transgenic plants that contain a heterologous gene wherein the heterologous gene comprises a sequence determined by a method as described herein, transgenic plants that contain a mutated gene wherein the mutated gene comprises a sequence determined by a method as described herein, tools for controlling gene expression, comprising a nucleic acid with a sequence obtained by a method as described herein and genes having a sequence prepared by any of the methods described herein. Other embodiments will be appreciated from a reading of the specification.

### DETAILED DESCRIPTION OF THE INVENTION

The inventors discovered methods and tools that, in most embodiments, avoid the use of regular negative or positive selection pressure to generate superior cell libraries of new DNA sequences. The term “regular negative or positive selection pressure” refers to gene selection that significantly affects cell survival enough for the gene to be used in selection procedures. In contrast, a “genetically neutral” gene desirably used for selection in the invention is not very essential to cell growth and survival and/or in preferred embodiments does not measurably affect survival.

The disadvantages of selection pressure on growth or replication are alleviated in embodiments of the invention by relying on an operator, reporter and/or separator gene product to distinguish cell clones of differing gene sequences without affecting cell survival or replication. These disadvantages include, among other things, an unacceptably high level of false positive and false negative clones. The disadvantages are particularly acute for larger libraries such as those having more than 1,000,000 members, as spontaneous mutations create more undesirable yet selected sequences at the higher population level. By using a negative or positive selection system any mutation that gives a selective advantage or disadvantage, respectively, may tend to accumulate and form a colony, and be falsely detected as having an operational gene variant. The methods discovered and presented here function intracellularly with natural transcriptional regulatory mechanisms that reflect functional DNA binding and thereby eliminate problems associated with extracellular DNA binding methods.

According to embodiments of the invention, the identification of DNA binding variants occurs unobtrusively to the cell and no particularly strong positive or negative consequences from the screening or selection mechanisms effects the growth or survivability of the cells. These properties of the invention, in vivo regulatory mechanisms from which no other significant positive or negative genetic pressures are created, have been found to be advantageous in minimizing or even in avoiding falsely identifying proteins that do not bind the desired DNA sequence.

In one embodiment, a target DNA binding sequence (a desired operator) is cloned adjacent to a structural gene used for screening and selection so that (1) the expression of the structural gene can be regulated through the binding of a DNA binding protein variant to the operator sequence, and (2) the DNA binding protein variants are expressed from DNA sequences that have been combinatorially mutated. In an advantageous embodiment the screening/selection gene(s) use reporter genes and/or separator genes that lack significant negative or positive evolutionary selective pressure for growth or survival of the cells.

The reporter and separator genes preferably are structural gene(s) that act to distinguish cells expressing the protein from cells having reduced expression of the gene. As used herein, a reporter gene codes for a “reporter” that is detectable either directly or indirectly. An example of a directly detectable reporter is a fluorescent protein such as green fluorescent protein. An example of an indirectly detectable reporter is an enzyme that is detected by addition of a substrate such as a calorimetric, fluorescent or chemilumigenic substrate. A cell can be separated from other cells based on detection of the expression level of reporter inside (intracellular) that cell or outside (extracellular to) the cell, or a combination of intracellular and extracellular.

As used herein, a separator gene codes for a protein that leads directly or indirectly to an altered molecular structure on the cell surface. Most typically the separator gene codes for a protein that goes to the outer surface and is found there. Separator gene expression allows physical separation of cells based on binding to the expressed molecule, which may be the separator protein, or something else which is influenced by the separator protein. A separator gene may, for example, be a antibody binding site, such as a single chain antibody, or an antigen. A cell (with its genetic complement) can, for example, be physically separated from other cells through specific binding with the separator gene product. Of course, combinations are possible that allow physical separation of cells based on the regulatory control of gene expression by the mutated DNA binding protein variant. In some cases a gene may be both a separator gene and a reporter gene. For example, a protein that has enzymatic activity yet is expressed at the cell surface can facilitate selection both by presenting a target for binding to the is the cell and by reacting with a suitable substrate to mark the cell in some manner, such as by formation of an optical product in the vicinity of the cell.

In another embodiment the gene expression levels of the reporter and/or separator genes are adjusted such that expression levels in the absence of binding of repressor protein to operator sequence are discernible from expression levels influenced by the binding of protein to the desired operator sequence. Still another embodiment is a method wherein lacZ and lacZ′ reporter gene product activities are assayed in vivo.

Another embodiment of the invention is the use of separator gene expression and repression through which clones containing the desired operator-binding protein variant are physically separated from those cells that do not contain such desired variants. In the final step, the expression and/or repression of a reporter or separator gene is used to finally select the cells that contain the desired DNA binding protein variants.

The selection and screening genes useful in this invention include any natural or synthetic gene or DNA sequence that encodes a peptide, protein or enzyme that can be detected or used to identify or separate cells expressing the product from those cells that have a repressed expression. Of special interest are genes that encode detectable products to distinguish or separate cells repressing or expressing the gene. Often these screening/selection genes should not be present or should not be intact in the host cell used for the screening experiment. Especially useful are genes encoding proteins or peptides that may act as antigens or ligands for monoclonal or polyclonal antibodies, enzymes that produce substances that are detectable by monoclonal or polyclonal antibodies, as well as gene products that are detectable directly or indirectly through chemical or physical reactions associated with them. By way of non-limiting example a gene product may create a colored chemical reaction product, something that consumes a colored reactant, or product(s) that are directly detectable. Especially preferred are reporter genes that encode proteins and enzymes that synthesize colored products, or that contribute significantly to the milieu required for a calorimetric reaction to proceed. The expression of the reporter gene may be enhanced by a method whereby the result can be visually or spectrophotometrically detected.

Of particular utility are gene products or gene fusion products that produce antibodies, fragments of antibodies, antigens, purification tags in the form of proteins, protein domains or peptides that can be expressed on the cell surface and that can be used to remove from a mixed culture those cells expressing such gene products or gene fusion products thereby enriching the remainder of the culture with cells that repress the expression of these genes. Preferred genes for the creation of such separation proteins that are under the transcriptional control of the to be identified DNA binding protein variants and that can be expressed and located to the E. coli outer membrane and are therefore of interest for separating repressed from non-repressed expression are the E. coli proteins lamB (maltoporin), K88 as and K88ad pilin proteins, TraT lipoprotein, PhoE, OmpA, OmpC, OmpF, OmpF, BtuB and the OmpA-lipoprotein fusion from Georgiou et al Trends Biotechnol. 11:6-10 (1993). Most advantageously the reporter and separator genes demonstrate no negative selective pressure for growth or survivability of the cell under the conditions used to discriminate expression from repression.

An example of a useful reporter gene is the Escherichia coli lacZ gene encoding β-galactosidase. In the proper medium, for example that contains the calorimetric lactose analog, Xgal (5-bromo-4-chloro-3-indoyl-β-D-galactopyranoside), the expression and repression of lacZ gene expression can be visually or spectrophotometrically discriminated. In the absence of a critical amount of β-galactosidase expression, i.e. under repressed lacZ gene expression conditions, Xgal remains (largely) unhydrolyzed and (largely) colorless. If however, lacZ gene expression is not repressed and β-galactosidase is sufficiently produced, Xgal is hydrolyzed to galactose and an indoxyl-derivative, the latter of which is then oxidized by air to a blue indigo dye that is easily detected visually or spectrophotometrically. Alternatively to using the entire lacZ gene as the reporter gene, one preferred embodiment uses the truncated version of this gene, the lacZ′ gene, together with the appropriate lacZAM15 mutated β-galactosidase gene expressed from the host cell chromosome in the process known as α-peptide complementation to achieve the same results.

Alternative examples and/or additions to the preferred lacZ and lacZ′ embodiments for the reporter genes (but not limited to these alternatives) include intrinsically fluorescent proteins like the green fluorescent protein and derivatives thereof and the luciferase enzyme.

The choice of a DNA binding protein gene as the starting point for the generation of DNA binding protein variants is in principle open to any DNA sequence that encodes an expressible protein. Advantageously, genes for producing DNA binding protein variants are known and may be used.

Regulatory DNA Binding Proteins

Regulatory DNA binding proteins can be categorized into at least four known major groups based on typical structures observed in the three dimensional representation of DNA binding proteins. Embodiments of the invention include the generation and/or modification and use of known proteins in each class. Embodiments of the invention include using known sequences of proteins of these classes and conserved amino acid substitutions from these sequences as starting sequences for new and useful binding proteins. Variations in the native sequence can be made using any of the techniques and guidelines for conservative and non-conservative mutations as for example set forth in U.S. Pat. No. 5,364,934.

A first class of proteins that are particularly useful for practice of the invention contain a motif called the helix-turn-helix motif (HTH, Brennen and Mathews, 1989; Pabo and Sauer, 1984) having α-helices that pack against one another. The helices are joined by a β-turn or a more extended loop structure, and have been observed to directly or indirectly interact with DNA sequences through side-chains of at least one of the helices. In general the helix-turn-helix motif is not a stable folding unit within the protein but is integrated into a 60 to 90 amino acid residue long domain. The protein structures outside of the HTH-motif within these domains may differ in structure from one HTH-containing protein to the next.

The structures of the HTH-motif, although displaying minimal amino acid homologies, often show similar relative positions of their α-carbon atoms. The second helix of the HTH motif (the recognition helix) is positioned in the domain such that this helix is adjacent to the major groove of the DNA. The side-chains of this recognition helix have been seen to interact with specific bases of DNA binding sequences through hydrogen bonding, so-called hydrophobic interactions as well as by complementary van der Waal's surface interactions. HTH binding motifs may exist in dimeric DNA binding proteins or in monomeric DNA binding proteins. Homodimeric DNA binding proteins possessing HTH motifs bind palindromic or partially palindromic DNA sequences. The HTH DNA binding protein motif and variations thereof can be found in both eukaryotic and prokaryotic organisms and is exemplified by such prokaryotic proteins such as λ cro, λ repressor, catabolite activating protein CAP, lac repressor, 434 repressor, 434 cro and others and by such eukaryotic proteins such as any of the homeodomain proteins like antennapedia, NK-2/vnd, and the POU-specific domain containing proteins and others.

A second class of DNA binding motifs is one in which one or more zinc ions is a structural component of the DNA binding domain, i.e., the zinc-containing DNA binding proteins. A typical motif of this class is the zinc-finger motif. In this motif, a zinc-ion is coordinated by cysteine residues or cysteine and histidine residues of the protein and results in a structure resembling a finger that interacts with the DNA in a sequence specific manner. DNA binding proteins possessing a zinc-finger motif are exemplified by Zif, EGR1, EGR2, GLI, Wilson's tumor gene, Sp1, Hunchback, Kruppel, ADR1 and BrLA proteins and others. Structural variations of the zinc-finger motif that also can be classified as zinc-containing motifs, with additional finger structures as exemplified by the glucocorticoid receptor or may contain binuclear zinc ion centers such as seen in the yeast GAL4 protein.

A third major class of known regulatory DNA binding proteins are proteins that contain a leucine zipper motif. This structural motif is involved in the dimerization of leucine zipper motif containing proteins. The leucine zipper motif generally comprises an α-helical structure having several leucine residues (typically up to five) spaced periodically through the helix (usually every seventh consecutive residue). This repeating structure within an α-helix results in orientation of leucine residue at a similar position on the face of the helix every second consecutive turn of the helix. The interface of two such juxtaposed leucine zipper helices from two separate polypeptide chains results in complementary hydrophobic interactions between the helices that can stabilize the protein dimer formed.

The leucine zipper class of DNA binding protein motifs is exemplified by several subclasses characterized by additional motifs within the subclasses. Examples of such subclasses of leucine zipper DNA binding proteins are the b/zip proteins GCN4, C/ERB, fos, jun, myc and others, the basic helix-loop-helix (b/HLH) proteins exemplified by the MyoD protein, and the basic helix-loop-helix zip proteins (b/HLH/zip) exemplified by the MAX protein.

A fourth, somewhat more diverse class of regulatory DNA binding proteins is characterized as having β-sheet structures that contribute to DNA binding. Examples of members from this group are the TATA binding protein (TBP), a general eukaryotic transcription factor that interacts with the minor groove of TATA box DNA through the factor's β-sheet structures, the prokaryotic Met repressor, the eukaryotic tumor suppressor p53 protein and the specific transcription factor NF-κB protein. As more three dimensional structures of DNA binding proteins are elucidated and reported it is likely that new classes and/or motifs for DNA binding proteins will be accepted.

DNA Binding Protein Genes

Advantageous embodiments of the invention utilize genes that code for DNA binding proteins that influence gene transcription. Particularly advantageous are genes or gene sequences that encode bacterial repressor proteins and/or fragments. Of the four different groups of DNA binding proteins enumerated above in this context, the DNA binding proteins that contain helix-turn-helix motifs are particularly preferred. However, it is also possible to use other sequences that encode zinc-containing proteins, leucine zipper containing proteins or members of other types of DNA binding proteins. Sequences of these proteins are known to skilled artisans and are not repeated here due to space restrictions. Embodiments of the invention include the use of known sequence from each class.

An advantageous embodiment of the invention uses a gene encoding a cro protein based on the homodimeric 434 cro protein and a second desirable embodiment uses a homeodomain based on the monomeric NK-2 homeodomain protein from the Drosophila melanogaster vnk gene. Of particular interest are DNA binding proteins from humans. In general, specific problems can be approached using species specific binding proteins. Accordingly, the methods encompass the use of specific animal and plant DNA binding proteins, as well as those from free-living as well as infective micro-organisms (including viruses). Because the desirable property of protein binding to DNA exists even in smaller portions of the protein, partial gene sequences which code for those portions also may be used.

The invention is particularly useful in the field of agriculture. The creation and use of DNA binding proteins that recognize specific DNA sequences such as, for example, transcription control molecular affecting virus genes, plant growth genes, senescence genes, fruiting genes, carbohydrate metabolism genes, and other genes, is particularly contemplated. Although the DNA binding activity of proteins as described herein often leads to decreased synthesis of one or more proteins, a skilled artisan will appreciate that increases in individual protein production also are possible. Examples of proteins that can be produced at increased levels utilizing the present invention include, but are not limited to, nutritionally important proteins; growth promoting factors; proteins for early flowering in plants; proteins giving protection to the plant under certain environmental conditions, e.g., proteins conferring resistance to metals or other toxic substances, such as herbicides or pesticides; stress related proteins which confer tolerance to temperature extremes; proteins conferring resistance to fungi, bacteria, viruses, insects and nematodes; proteins of specific commercial value, e.g., enzymes involved in metabolic pathways, such as EPSP synthase. DNA encoding regulatory elements and encoding protein are known to the skilled worker in that field, as exemplified by U.S. Pat. No. 5,702,933, issued to Klee et al., and other representative citations in that publication.

Regulation Through Binding Between Protein and DNA

Embodiments of the invention utilize binding between protein and DNA. As will be appreciated by a skilled artisan, a variety of binding interactions have been discovered and are useful for these embodiments.

A DNA target or other DNA according to embodiments of the invention include not only the specific sequence listed but also similar sequences that are homologous to the sequence. DNA homology is determined routinuely by a skilled artisan. By way of example, a DNA sequence that is 50% homologous to a cognate binding sequence of 8 base pairs long will have an identical match for any 4 of the bases when the two sequences are lined up side by side.

Through the work mainly of Stephen Harrison and coworkers who determined the three-dimensional structure of the cro repressor protein from the bacteriophage 434 (434 cro), it is known that this DNA binding protein is made up of two identical monomers comprised of 71 amino acid residues each that are folded into a single domain having 5 α-helices. Helices 2 and 3 (numbered from the N-terminus to C-terminus) form HTH motifs, with the first and fourth helices packing against the HTH to create a hydrophobic core. Interactions between the monomers are formed by protein-protein interactions from structures of the C-terminal end of the monomers, specifically in helices 4 and 5 and loop structures between helices 3 and 4 (Mondragon et al, 1989; Harrison and Aggarwal, 1990; Mondragon and Harrison, 1991 and Padmanabhan et al., 1997). As with other HTH proteins, the second helix of the each of the HTH motifs of the monomers (helix 3 of 434 cro) is found to sterically fit into the major groove of the DNA binding sequence. As with other homodimeric HTH proteins, the two recognition helices of the homodimer are separated by a distance that allows them to fit into the major grooves of a consecutive turn of the DNA double helix.

The specific DNA sequences that bind with highest affinity to wild-type 434 cro protein form the operators of a regulatory genetic switch that participate in the regulation of lytic or lysogenic life-cycles of the bacteriophage (Ptashne, M. The Genetic Switch). These operators are named OR1, OL1, OR2, OL2, OR3 and OL3 from their positions within the bacteriophage genome. The cro protein from 434 binds the OR3 operator sequence with highest affinity, followed by that of the OR1 sequence. The specific operator control sequences for 434 cro are partially palindromic DNA sequences of approximately 14 base pairs in length that to varying degrees possess palindromic base sequences in the first and last four bases of the operator DNA. The consensus sequence for the palindromic part of these operators is 5′ ACMNNNNNNTTGT-3′ (where N is a nonpalindromic base). The OR3 operator is an exception to the palindromic consensus and possesses a single 5′-ACAG-3′ half-site (Koudelka and Lam, 1993; Bell and Koudelka, 1995).

In the 434 cro OR1 DNA complex solved by X-ray structural analysis by Harrison and coworkers, one can observe at high resolution a multitude of protein-DNA interactions responsible for the high-affinity and specificity of cro for operator OR1. Each of the cro monomers is secured across the major groove of the DNA by a network of contacts between the sugar-phosphates of the DNA and protein-imido, protein-guanidinyl and protein amino groups. The HTH motif is anchored across the major groove by interactions of the amino-terminus of helix 2 on one side of the major groove and through the turn and the loop between helices 3 and 4 with the other side of the major groove. These interactions position the amino-end of the recognition helix to allow several side-chain interactions between residues of the recognition helix and specific bases of the operator DNA sequence. The surfaces of the amino end of the recognition helices that face the DNA of the major groove form complementary binding surfaces with the DNA operator regulatory sequence.

Interactions through these complementary surfaces include hydrogen bonding between amino acid residue side chains and bases of the DNA binding sequence, hydrophobic interaction surfaces, van der Waal's surface complementarity and ionic interactions between protein and DNA of the operator. The DNA in the complex is bent with respect to standard B-DNA. Within the HTH, lysine 27 and serine 30 interact with the sugar phosphate backbone of the operator. Glutamine 28 can form one or two hydrogen bonds between its sidechain amide carboxyl group and the N6-amino of the adenine base of the first operator base-pair and/or an amide NH and the lone pair of the N7 of adenine 1. The second residue of the recognition helix, glutamine29 can from a hydrogen bond with the 6-oxa group of the guanine base of operator base-pair two. Base pair three contacts with the protein are of an hydrophobic nature with the thymine methyl group fitting a pocket constructed from the methylene groups of the side-chains of Iysine27 and glutamine29. Three residues of the recognition helix, glutamine 29, serine 30 and leucine 33, are in van der Waal's contact with base-pair four of the OR1 operator. Base-pair 4 is the nonconsensus base-pair of the OR3 operator and is therefore implicated in both binding specificity and affinity differences between 434 repressor and 434 cro proteins.

The central base-pairs of the operator sequence do not contact the HTH motif of 434 cro. Binding specificity of the 434 cro protein and HTH proteins in general seem to be governed by the amino acid sequences of the recognition helices and the specific interactions between these residues with bases of the DNA binding sites. Conformation of the DNA and orientation of the protein on the DNA however, are also thought to be major determinants of DNA binding affinity and specificity and these attributes remain unpredictable. Although some workers have proposed a specificity code for at least one member of the HTH DNA binding proteins (Lehming et al, 1990) most of the specificity interactions between HTH containing proteins and their cognate DNA binding sites are too complex to be predicted.

Several biochemical and genetic investigations have examined the influence of amino acid sequence residues in the HTH motif on DNA binding sequence specificity. Some of these studies have shown that substitution of single amino acids causes altered DNA binding specificity. For example, Caruthers et al. (1987) examined the effects of rationalized changes in the protein DNA interface of λ cro and its OR1 operator and variants of OR1, and identified one mutant with altered specificity. Wharton and Ptashne (Nature 1985, 316:601-605) showed that substitution of glutamine for alanine at the first residue of the recognition helix of 434 repressor resulted in a mutant repressor with altered base preferences for the first basepair of the operator.

Others, for example, Ebright et al (1984) working with the CAP protein, and Spiro and Guest (1988) working with the FNR protein, have similarly shown DNA binding changes as a consequent of mutations in the recognition helices of the respective proteins. Amino acid substitutions of the first two residues of the recognition helices of the 434 and lac repressors resulted in altered DNA binding in the mutant proteins (Wharton and Ptashne, 1987; Lehming et al. 1990). Others have not had remarkable success in changing the binding specificity of HTH proteins (Huang et al. 1994) and it is noteworthy that the selection method used was one that depended on the ability of the DNA binding protein variant to inhibit a process that was deleterious for the cell. Still others have recently had moderate success in changing specificity of binding of HTH proteins. Simoncsits et al 1999 for example successfully identified a single chain 434 repressor variant that preferred a mutant DNA binding sequence half-site with one out of four bases altered.

Targeted DNA Sequences

Target DNA, in many embodiments are regulatory sequences which interact with DNA binding protein(s) to cause a change in gene expression. A few examples of such sequences include genes that are substantial or essential for the establishment or maintenance of a disease or disease state, i.e., a gene essential for an infectious state, a toxin, and/or the survival and/or replication of the causative agent of the disease, or genes which encode various traits and/or functions of plants, animals or other organisms.

Causative agents of disease are microorganisms such as viruses, bacteria, parasites like trypanosomes, protozoan, and plasmodia as well as higher organisms and including cells of the human body, especially those that are of a degenerative, transformed or have otherwise undesirable traits or characteristics, such as those of malignant or benign tumors, lymphomas, myelomas, carcinomas, plant viroids and the like.

Advantageous target sequences are those that are evolutionarily conserved, highly conserved or relatively highly conserved, for examples of the latter, sequences of the HIV-1 long terminal repeat regions in general and U3 region in particular. Of particular interest are target sequences from human immunodeficiency virus types 1 and 2, human papilloma viruses, breast, prostate, ovarian, liver, lung, spleen, muscle, cancer cells, plant viruses, plants and the like. Palindromic or partially palindromic target sequences are preferred when the desired DNA binding protein variant is a member of the homodimeric proteins. Nonpalindromic target sequences are preferred when a monomeric or heterodimeric DNA binding protein variant is desired.

Balanced Gene Expression

In embodiments of the invention, a target sequence is cloned into a position adjacent to a reporter gene and/or separating gene such that the target can then function as an operator sequence for the regulation of the gene expression in for example a bacterial system using a DNA binding protein as repressor. Most importantly for embodiments of the invention the gene (both reporter or separating) is genetically neutral. That is, a protein gene is chosen such that, upon up-regulation or down-regulation does not strongly affect cell growth or survival. Examples of such genes include such reporter genes as lacZ and lacZ′ derivatives, intrinsically fluorescent proteins such as the green fluorescent protein and derivatives thereof and the luciferase enzyme, and separator genes such as the E. coli proteins lamB (maltoporin), K88 as and K88ad pilin proteins, TraT lipoprotein, PhoE, OmpA, OmpC, OmpF, OmpF, BtuB and the OmpA-lipoprotein fusion from Georgiou et al Trends Biotechnol. 11:6-10 (1993), Strep-tags (protein sequence, W “X” H P G F “Y” “Z”, in which “X” represents any desired amino acid and “Y” and “Z” either both denote Gly, or “Y” denotes Glu and “Z” denotes Arg or Lys), His-tags (sequences composed of a minimum of 5 consecutive HIS residues), FLAG-Tag protein epitope sequence (protein sequence DYKDDDK, TP Hopp, K S Prickett, V Price, R T Libby, C J March, P Cerritti, D L Urdal, P J Conlon. BioTechnology 6:1205-1210, 1988), the HA epitope (protein sequence YPYDVPDYA, H L Niman, R A Houghten, L A Walker, R A Reisfeld, I A Wilson, J M, Hogle, R A Lerner. Proc. Natl. Acad. Sci. USA 80:4949-4953, 1983; I A Wilson, H L Niman, R A Houghten, M L Cherenson, M L Connolly, R A Lerner. Cell 37:767-778, 1984), the c-myc epitope tag (protein sequence EQKLISEEDL, S Munro, H R B Pelham. Cell 48:899-907, 1987), AU1 (protein sequence DTYRYI) and AU5 (protein sequence TDFYLK) epitopes (PS Lim, AB Jenson, C Consert, Y Nakai, L Y Lim, X W Jin, J P Sundberg. J. Infect. Dis. 162:1263-1269, 1990; D J Goldstein, R Toyama, R Dhar, R Schlegel. Virology 190:889-893, 1992), the Glu-Glu epitope (protein sequence EEEEYMPME, T Grussenmeyer, K H Scheidtmann, M A Hutchinson, E Eckhart, G Walter. Proc. Natl. Acad. Sci. USA 82:7952-7054,1985; B Rubinfeld, S Munemitsu, R Clark, L Conroy, K Watt, W Crosier, F McCormick, P Polakis. Cell 65: 1033-1042,1991), the KT3 epitope (protein sequence PPEPET, H MacArthur, G Walter. J. Virol. 52:483-491, 1984; G A Martin, D Viskochic, G Bollag, P C McCabe, W J Crosier, H Haubruck, L Conroy, R Clark, P O'Connell, R M Cawthon, M A Innis, F McCormick. Cell 63:843-849, 1990), the IRS epitope (protein sequence RYIRS, T C Liang, W Luo, J T Hsieh, S H Lin. Arch. Biochem. Biophys. 329:208-214,1996; W Luo, T C Liang, J M Li, J T Hsieh, S H Lin. Arch. Biochem. Biophys. 329:215-220,1996), the BTag epitope (protein sequence QYPALT, L F Wang, M Yu, J R White, B T Eaton. BTag: Gene 169:53-58, 1996), the Protein Kinase C epsilon (Pk) epitope (protein sequence KGFSYFGEDLMP, Z Oláh, C Lehel, G Jakab, WB Anderson. Anal. Biochem. 221:94-102, 1994) and the Vesicular Stomatitis Virus (VSV) epitope (protein sequence YTDIEMNRLGK, T Kreis. EMBO. J. 5:931-941, 1986, J R Turner, W I Lencer, S Carlson, J L Madara. J. Biol. Chem. 271:7738-7744, 1996)

Accordingly, in embodiments of the invention, genes that encode the following proteins are particularly desirable for separators and/or reporters as being genetically neutral: lacZ, lacZ, green fluorescent protein, luciferase, lamB, K88 as pilin, K88ad pilin, TraT, PhoE, OmpA, OmpC, OmpF, OmpF, BtuB, OmpA-lipoprotein fusion, Strep-tag, His-tag, FLAG-Tag epitope, HA epitope, c-myc epitope, AU1 epitope, AU5 epitope, Glu-Glu epitope, KT3 epitope, IRS epitope, BTag epitope, protein kinase C epsilon (Pk) epitope, the Vesicular Stomatitis Virus (VSV) epitope, bacteriophage M13, fd or f1 gene VIII protein and gene III protein.

Genes that are to be avoided, because they tend to impart genetic selection advantage under many circumstances are: Toxins, such as those produced from the S, R and Rz genes of bacteriophage lambda, the gene E protein from bacteriophage phi-x 174, nutritional and chemical resistance genes, genes that metabolize growth inhibitory substances to substances that do not inhibit growth and vice versa, genes that determine a resistance to lytic bacteriophage infections, for example, antibiotic genes, galT,K, tetA, lacZ+ (when used to generate toxic metabolites), pheS, argp, thyA, crp, pyrF, ptsM, secA, maIE, ompA, btuB, lamB, tonA, cir, tsx, aroP, cysK, and dctA. The combination of promoter sequence, target operator sequence and choice of reporter gene and separator gene used in specific experiments affects the strength of expression of the reporter gene and separator gene. The strength of the reporter and/or separator gene expression also may vary as assayed, for example, by the enzymatic activity of the reporter gene itself or by the quantity of separator gene product available on the outer surface of the cell for binding to the separating medium. Accordingly, it is desirable for optimizing identification of cells exhibiting a repressed phenotype due to a DNA binding protein variant binding to the target operator sequence, to balance the strength of gene expression levels with the specific reporter and/or separator genes as well as with the operator used. One exemplary embodiment for the discovery of balanced gene expression of the reporter gene and/or separator gene for the identification of cells having repressed phenotypes for these genes utilizes, in preliminary experiments, combinatorial mutagenesis of the minimal promoters used for reporter gene and/or separator gene transcription.

Additionally or alternatively, combinatorial mutagenesis of the sequences of the ribosomal binding site through the start codon used for reporter gene and/or separator gene translation can be utilized. In these embodiments, cells expressing a reporter gene and/or separator gene construct that has mutated transcriptional or translational control sequences that are expressed in the unrepressed states are compared to cells containing optimally balanced promoter-target operator-translational control sequence-reporter and/or separator gene constructs for reporter gene and separator gene expression. Such gene expression is comparably assayed in an advantageous embodiment through, for example, the analysis of reporter gene activity measurements and separator gene product surface expression.

In other embodiments, mutagenized separator gene constructs are assayed for their ability to bind separator medium similarly and for the ability to be released from separator medium similarly to known and balanced separator gene expression. The identification of such balanced gene expression in newly created constructs is important for the optimal identification of repressed phenotypes.

The identification of DNA binding protein variants that bind to the desired target operator sequences and not to sequences unrelated to the desired target sequences can be improved by design considerations of the genetic constructions used. The target operator DNA sequences need to be placed within a maximal distance from the +1 position of the promoter so that repression of transcription will be achieved.

For homodimeric DNA binding proteins that bind palindromic or partially palindromic DNA sequences, partially palindromic sequences in the promoter regions of the reporter and separator gene constructions other than those present in the target operator sequences need to be avoided. These palindromic or partially palindromic sequences can be detected by visual inspection or by appropriate computer analysis of the sequences in question. For nonpalindromic desired target DNA sequences, the monomeric DNA binding protein variants can be directed to the approximate location of the desired operator by fusion of the coding sequences of the mutagenized DNA binding protein with a second DNA binding domain having a known binding sequence specificity that differs from the desired target specificity. This known specificity of the second domain should be of a reduced affinity such that repression of the reporter gene and/or separator genes does not occur by the second domain when used alone. In this embodiment, the desired target operator is cloned adjacent to the known operator of the second DNA binding domain. The known operator should optimally be more than 10 basepairs away from the +1 position of the promoter used for reporter and separator gene transcription. In this embodiment, the variants of the mutagenized DNA binding protein that bind the desired target operator sequence are assisted to the desired target sequence by the second domain binding to its binding sequence. Variants that bind with high affinity to the desired target DNA sequence are found that repress reporter and separator gene expression. Site directed or cassette mutagenesis techniques that induce mismatches in the known DNA binding sequence of the assisting domain can be used to reduce, balance or otherwise achieve optimal repressible transcription activities.

In an advantageous embodiment, a DNA sequence encoding a DNA binding protein is then mutated so that a large collection of different mutations and combinations of mutations are generated. Different collections of mutations and combinations of mutations can be constructed in specific regions of the protein known to have an influence on the DNA binding properties of the protein as well as in regions not directly known to have an influence on DNA binding. Each of these collections is for the purposes of this description of invention termed a combinatorial mutational library. These mutational libraries can be constructed such that they have varying complexities, from several tens of thousands of mutations and combinations of mutations to millions or billions of such combinations.

By the expression of such mutational libraries, a multitude of different DNA binding protein variants are created that can bind different DNA sequences with differing binding affinities. Those mutations and combinations of mutations that are able to bind to the target operator DNA sequences can thereby regulate the expression of the adjacent reporter or separator gene by influencing transcription. A quantity of the separator gene product may be expressed on the cell surface and can bind a component of the separation media. Through this binding a cell that expresses unrepressed or non-transactivated levels of separator gene product on its surface may be removed or separated from those displaying repressed or transactivated expression. Thus, cells that contain DNA binding protein variants that bind the desired DNA binding sequence are selected through the resultant activity of the reporter gene product on the final cell culture of cells enriched for repressed or transactivated reporter gene and separator gene phenotype(s).

In an advantageous embodiment, a separator gene product may be chosen from surface proteins or coat proteins of bacteriophage or other viral genomes. When the transcriptional expression of such a separator protein is regulated by a DNA binding protein variant or other transcriptional regulatory protein and when the regulatory protein is encoded by the bacteriophage, phagemid or viral genome, then a quantity of separator gene product may be expressed on the surface of the bacteriophage, phagemid or virus and can bind a component of the separation media. Similar to the description above, through this binding a bacteriophages, phagemids or viruses that are replicated in cells having unrepressed or non-transactivated levels of separator gene product may be removed, enriched or separated from those replicated in cells having repressed or transactivated expression. Bacteriophages, phagemids or viruses that encode DNA binding protein variants that bind the desired DNA binding sequence are selected through the resultant activity of the reporter gene product on the final cell culture of cells infected with a population of bacteriophages, phagemids or viruses enriched for repressed or transactivated reporter gene and separator gene phenotype(s).

Linkage of Reporter and Separator Genes

An advantageous embodiment of the invention has the reporter and separator genes cloned together as an operon with the target selection DNA binding sequence and minimal promoter sequence on one plasmid vector. A DNA binding protein that is mutated into a combinatorial library on a second plasmid vector is expressed together with the first plasmid in a bacterial cell. The plasmids then are transformed sequentially into a host cell where preferentially, the separation reporter gene plasmid is first transformed into the host cell followed by transformation of the resultant cells with the combinatorial library expressing the DNA binding protein variants. The host cell is any cell that can replicate and express the reporter gene, separator gene and DNA binding protein variants and that is capable of showing a repressed phenotype for the reporter gene and separator genes. An advantageous host cell is the Escherichia coli strain DH5α (Life Technologies, Inc., Gaithersburg, Md.).

The protein-coding sequence of the DNA binding protein, herein named the regulator gene, can be mutated via several known methods. These methods can be random or may use targeting to specific regions of the regulator gene. Especially preferred as an embodiment of this invention are in vitro mutagenesis methods. In these methods, isolated DNA composing parts or all of the DNA binding protein can be mutagenized at specific positions within the gene. Especially preferred for the mutagenesis are the use of mutagenic DNA-cassettes.

In principle, a regulator gene can be modified by the insertion of additional nucleotide residues, especially in form from chemically synthesized oligonucleotides, as well as the deletion of nucleotide residues from the gene, as well as the incorporation of point mutations within the regulator gene. Combinations of multiple additions and/or deletions and/or point mutations can also be incorporated in the regulator gene.

A preferred embodiment of this invention uses combinatorial libraries of mutations of the regulator gene. In principle, in the in vitro mutagenesis reactions, single stranded DNA can be synthesized with many mutations and combinations of mutations within the coding sequence of the regulator gene. Single stranded and/or double stranded DNA can alternatively be enzymatically, chemically or physically treated such that mutations and combinations of mutations within the coding sequence of the regulator gene are created. Through the hybridization of oligonucleotide primers to the single-stranded or denatured double-stranded, mutagenized DNA and the use of in vitro DNA polymerase reactions for the conversion of the oligonucleotide-primed/mutagenized DNA hybrid molecules to double-stranded DNA molecules.

The sequences of interest from the mutagenized double-stranded DNA molecules so created can then be hydrolyzed from the DNA polymerase reaction products through the use of appropriate restriction endonucleases and are thereby made available for use in subsequent cloning experiments. In a preferred embodiment, these subsequent cloning experiments combine the so-mutagenized and restricted, mostly double-stranded DNA molecules that encode variants of the sequence of interest of the DNA binding regulator protein into a cloning vector containing the remaining parts, if any, of the DNA binding regulator protein such that the expression of the DNA binding regulator variants as proteins is assured.

The resultant regulator DNA binding protein variants that bind a specific DNA sequence or sequences can be genetically fused to DNA sequences known to help activate or repress the transcription of a gene to be regulated in other cell types.

In one embodiment protein genes that encode zinc-finger DNA binding motifs may be modified. Preferably, libraries of altered proteins that bind DNA sequences are made based on known techniques for genetic manipulation. Such proteins and their DNA binding motifs which are known or that may be discovered in the future may be utilized as starting material for embodiments of the invention. For example, U.S. Pat. Nos. 6,013,453 and 6,242,568 show DNA sequences of mutational libraries that encode zinc-finger DNA binding motifs for new DNA binding proteins that bind to desired DNA regulatory sequences. The DNA mutational libraries of these zinc-finger protein variants can be used to identify protein species that bind to specified DNA sequences. A randomized library of zinc-finger sequences may be examined by binding with one or more DNA sequence triplets. In this case, randomized zinc-fingers may be positioned between, or next to, two or more zinc-fingers that have defined sequence and binding specificities. These procedures can determine preferred target DNA sequences for the randomized fingers. In this way, new zinc-finger proteins with having multiple fingers can be constructed with novel specific DNA sequence binding characteristics and are useful for practice of embodiments of the invention. The sequences, materials and methods taught in these patent specifications are particularly included by reference.

Linkage by Fusion for Transformation and Subsequent Use

In one embodiment of the invention, such DNA sequence specific binding variants are fused to transcriptional activator domains important in the activation of prokaryotic and especially eukaryotic transcription. In a second preferred embodiment DNA sequences coding for protein domains associated with the inhibition of DNA transcription can be genetically fused with the DNA binding specific protein variants so that transcription of genes for example in eukaryotic cells can be repressed. Additional DNA sequences that encode protein domains or signal sequences useful for the targeting of a protein to a certain cellular compartment including extracellular compartments, can be fused to the resultant protein-encoding sequences.

An important therapeutic use is, for example the cloning of DNA sequences that encode regulator protein variants that are discovered with these methods and that bind to DNA sequences found in the long terminal repeat region of HIV-1 and additional fusions of these regulatory variants in hematopoietic stem cells. A multitude of such regulator variants can be used that recognize many different variations of these long terminal repeat sequences that could arise by mutation of the long terminal repeat DNA sequences of the HIV-1 virus.

When the so modified hematopoietic stem cells are returned to a patient and allowed to mature, the immune cells will recognize the proteins made by these genetically changed stem cells and lymphocytic stem cell descendents as “self”. If an HIV-1 virus infects such a genetically-modified lymphocyte, then the transcription of the viral genome and or parts thereof that are dependent on the HIV long terminal repeat sequences will be inhibited due to the presence of the long terminal repeat DNA sequence-specific transcriptional repressor protein(s). The replication of the virus will be thereby inhibited. The so-modified lymphocytes will remain viable and active and will be further available for immune function. A further use of the invention is the use of proteins or protein domains derived from the so-identified DNA-binding specific regulators as therapeutic agents.

Another important potential therapeutic use for like regulators that are discovered with the invention and that are specific for DNA sequences important for the induction/transformation and/or maintenance of carcinoma and pre-carcinoma states in cells infected by human papilloma virus types (HPV) would be as inhibitors of transcription from such sequences. Transduction of cells of the cervix and neighboring tissues with gene transfer vectors containing DNA sequences that encode such regulator variants that bind and regulator variants fused with transcriptional repression and other domains would prevent the transcription of the genetic information that contributes to such cancerous and pre-cancerous states. The repression of these HPV genes should inhibit both the replication and spread of the HPV infection as well as the induction of pre-cancerous and cancerous states. A further use of the invention is the use of proteins or protein domains derived from the so-identified DNA-binding specific regulators as therapeutic agents.

A further important potential therapeutic use of the invention is, for example, the identification of regulators that inhibit the expression of genes that are essential for tumor growth or survival or that activate the expression of tumor-suppressor genes or genes that activate cell death—apoptosis programs. The DNA encoding such regulators for tumor genes or tumor suppressor genes can be delivered to the tumor cells by gene delivery systems of viral or nonviral types or of microbiological nature. The expression of such genes within the tumor cells of the patient should inhibit the growth and replication of the tumor cells. A further use of the invention is the use of proteins or protein domains derived from the so-identified DNA-binding specific regulators as therapeutic agents.

A further important use of the invention is the creation and identification of regulators of gene expression, either as repressors or activators, for genes of interest in the fields of remediation and therapeutics for biodefense and emerging diseases. In this area, it is of interest to identify and use regulators of genes that are responsible for pathogenesis or virulence in naturally occurring or genetically modified biological agents that may be used in terrorism or warfare or that may emerge in the population by natural mechanisms. Several such agents, such as those responsible for Staphylococcal infection, ssmallpox, tularemia, Q-fever, anthrax, Venezuelan equine encephalitis, plague, botulism, smallpox, glanders and Marburg and Ebola viruses, have been exploited for their pathogenic traits (Abilek and Handelman, 1999; Broad et al., 2001). Other agents such as other orthopox viruses including monkeypox and camelpox pose important emerging medical threats. Inhibition of gene expression that is essential to the replication, virulence or pathogenesis of these agents would likely attenuate their pathogenesis. For example, protein variants that inhibit expression from promoters in Bacillus anthracis responsible for the expression of genes essential for pathogenicity (for example, atxA, pagA, lef and cya and capB) might prove useful in therapeutic treatments for anthrax. Similarly, inhibition of expression of genes found to be essential for replication or virulence in diseases caused by orthopox viruses may well prove very useful. For example inhibitors of expression of the promoters in the variola major India variant H4L, M1R, F6R, H8R, C14L, N1L, F4R that are very highly conserved (100% identity in up to 13 sequenced orthopox viruses) essential genes for smallpox replication or virulence might prove useful in attenuating pathogenicity from a broad range of these agents.

A further important use of the invention is the creation and identification of regulators of gene expression, either as repressors or activators, for genes of interest in what has become known as target validation studies. In these studies, it is of interest to identify and use such regulators for the repression or activation of genes and gene products that are of interest to the pharmacological industry. By the use of such regulators in cell and organism studies, the influence of the repression or activation of the specific gene under study on other related and unrelated genes and gene products can be observed. Such observations can take the form of for example genome-wide, or selective gene set expression studies, for example through DNA array technologies, through northern or western analyses, and through other such technologies.

A further use of the invention is in the area of target discovery studies. In such studies, combinatorial libraries of DNA binding protein domains of repressor or activator regulatory genes can be inserted using molecular biological gene transfer methods into cell or other assay systems that have phenotypes that are desired to be affected. The action of specific repressor or activator construction variants is compared using the phenotype of interest to control experimental cells not having a DNA binding domain in the otherwise identical regulator construction. Cells displaying a DNA binding protein variant-dependent desired change in phenotype are investigated further. The specific DNA binding protein variant responsible for the phenotype change is isolated and its gene is sequenced. The effects of the specific variant are then characterized using genome-wide, or selective gene set expression studies, for example through DNA array technologies, through northern or western analyses, and through other such technologies in order to discover the gene(s) responsible for the phenotypic changes.

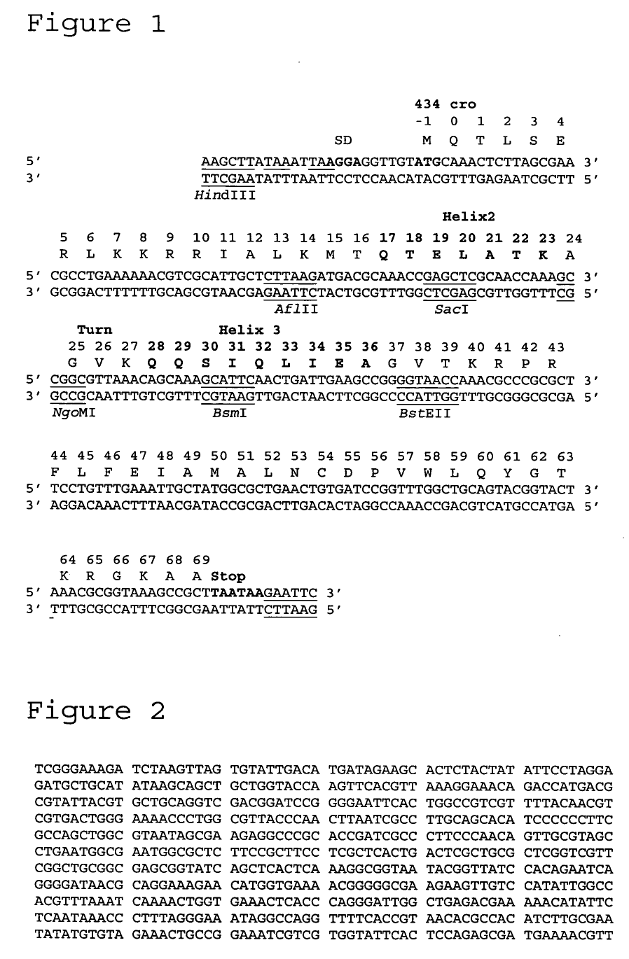

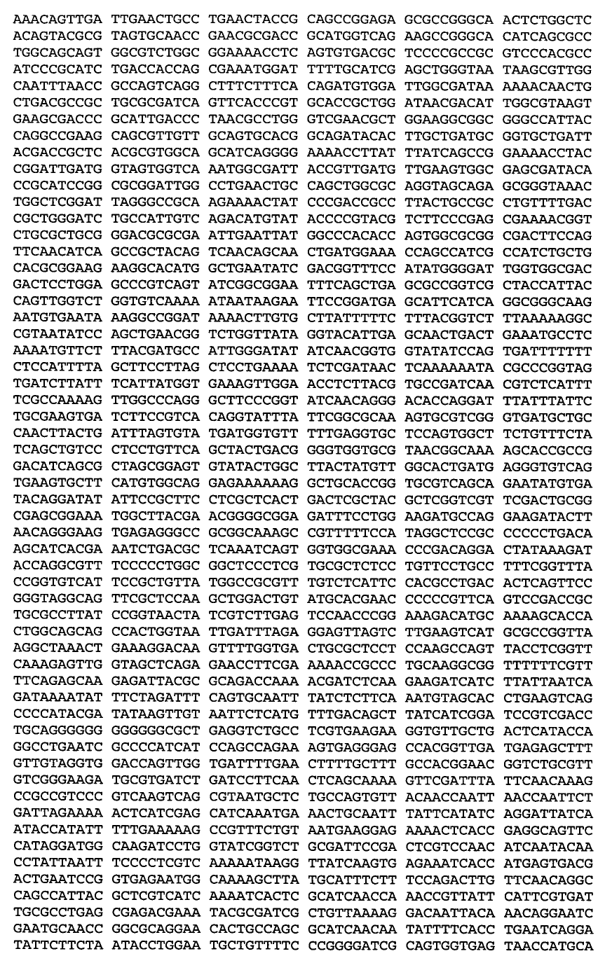

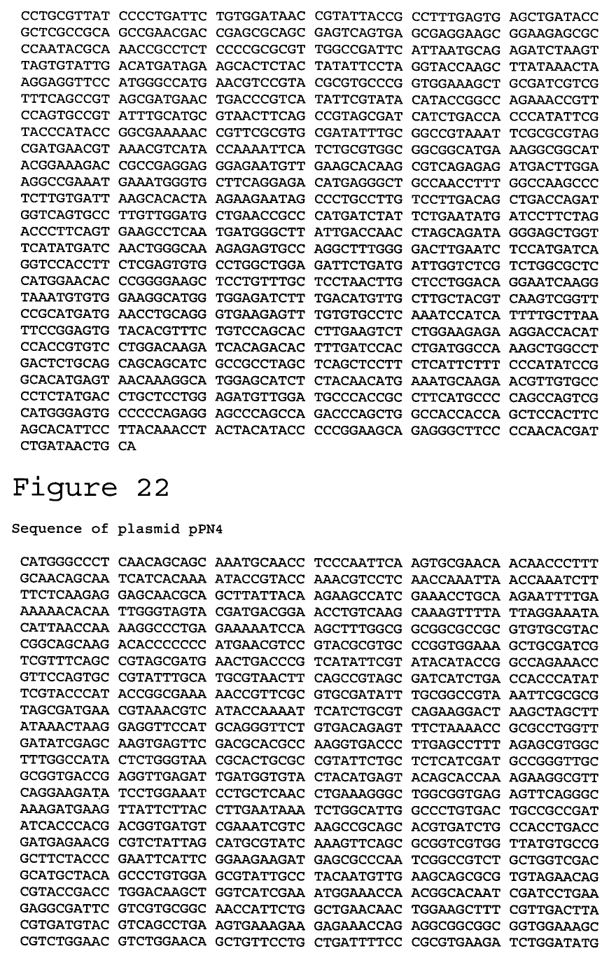

The invention also encompasses a kit for the construction and identification of such DNA sequence specific DNA binding protein variants. The kit contains a reporter/separator gene plasmid as well as DNA binding protein expression plasmids and mutational cassettes for the construction of mutational libraries of the DNA binding protein (see FIGS. 1 through 23).

The invention is further illustrated by the following examples, which are meant to illustrate embodiments and not to limit the claims in any way.

### EXAMPLES

### Example 1

### Use of a Screening Plasmid with a lacZ-Derived Reporter Gene for the Identification of 434 cro Variants that Bind HIV-1 Target Sequences

A. Description of the Promoter-Target operator-LacZ-Reporter Plasmid, pP2HIV1:

Plasmid pP2HIV1 was constructed from synthetic DNA, and from DNA derived from the vectors, pACYC184, pUR222, pUC119 and pUC4KAN. This plasmid was used to screen 434 cro DNA binding protein variants expressed from a repressor plasmid library (described below) to DNA target sequences derived from HIV-1 DNA (GenBank Sequence AF096643.1, bases 373 to 394) in in vivo screening experiments.

A synthetic double stranded oligonucleotide cassette was created from oligonucleotides having the following 5′ to 3′ (upper strand) DNA sequence (SEQ ID NO: 1):

5′TCGGGAAAGATCTAAGTTAGTGTATTGACATGATAGAAGCACTC
TACTATATTCCTAGGAGATGCTGCATATAAGCAGCTGCTGGTACCA
AGTTCACGTTAAAGGAAACAGACCATGACGCGTATTACG-3′.

The first base of this sequence is arbitrarily assigned the base number 1 of the pP2HIV1 plasmid. This cassette encodes a BglII restriction site followed by an optimized transcription promoter, an Styl restriction site, the HIV-1 target sequence, a KpnI restriction site, a 13 base pair spacer, an optimal Shine-Dalgarno ribosome binding site (AGGA) followed by an 8 base pair spacer and a translation initiation start sequence (ATG). The synthetic cassette in pP2HIV1 is followed by 12 base pairs of protein coding DNA (5′ACGCGTATTACG3′) that is fused to 22-basepairs of lacZ′-derived DNA from the vector pUR222 (bases 1857 to 1835 of GenBank sequence L09145.1). This DNA is followed in pP2HIV1 by additional lacZ-derived DNA from vector pUC119 (GenBank sequence U07650.1, bases 285-451). The pUC119 derived DNA of pP2HIV1 is then followed by 1941 base pairs of DNA derived from pACYC184 (GenBank Sequence X06403.1, bases 3946 to 4245, base 1 to 1521). The pACYC184-derived DNA of pP2HIV1 is fused to the kanamycin resistance region of vector pUC4KAN (GenBank sequence X06404.1, bases 404 to 1673).

The synthetic P2 promoter region of pP2HIV1, bases 8-52 was optimized for screening blue white phenotypes using 434 cro-derived repressors in E. coli DH5a using Xgal containing media IM2. The IM2 medium contained per liter 10 g bactotrypton, 2 g yeast extract, 5 g NaCl, NaOH to pH 7.0, 12 g Agar, 0.8 ml 50 mg/ml ampicillin, 1.0 ml 30 mg/ml kanamycin, 2.5 ml 2% 5-bromo-4-chloro-3-indolyl-□-D-galactopyranoside previously dissolved in dimethylformamide and 0.5 ml 1M isopropyl-□-D-thiogalactopyranoside. The promoter of pP2HIV1 can be removed and replaced by other synthetic promoters with other characteristics using the unique BglII and Styl restriction sites. These sites facilitate the combinatorial mutagenesis of the promoter for the purpose of selecting optimal promoter characteristics with specific target DNA sequences. The target DNA sequences can be synthesized from synthetic oligonucleotides and can be exchanged using the unique Styl and KpnI sites of pP2HIV1.

An additional screening vector for use in control experiments, pP2null, was constructed by digesting pP2HIV1 DNA with Styl and KpnI followed by ligation in the presence of a single stranded linker oligonucleotide having the sequence, 5′CTAGGTAC3′. Plasmid pP2null is identical in sequence with plasmid pP2HIV1 except that the HIV1-derived target sequence of pP2HIV1 has been deleted.

B. Description of the 434 cro expression vector, p434cro2:

Plasmid p434cro2 was used to create combinatorial mutation libraries of the 434 cro gene and to express these protein variants in E. coli cells containing the pP2HIV1 screening plasmid. Plasmid p434cro2 is based on the pUC119 cloning vector (GenBank sequence U07650.1).

Plasmid p434cro2 was constructed as follows. A synthetic gene encoding a Shine-Dalgarno ribosome binding sequence followed by a 434 cro protein encoding sequence optimized for expression in E. coli was synthesized from four oligonucleotides. The gene included unique restriction sites for the replacement of the DNA encoding the HTH region of 434 cro and was made double-stranded using a T4 DNA polymerase reaction, restricted with HindIII and EcoRI and cloned into HindIII and EcoRI-digested pUC119 DNA. The synthetic 434 cro gene has the sequence shown in FIG. 1 (SEQ ID NO: 2).

In order to simplify analysis of alpha complementation activity using the lacZ′ gene of plasmid pP2HIV1 in the presence of the p434cro2 plasmid, the partial coding sequence of the lacZ′ gene of the latter was removed by EcoRI and Kasl digestion, followed by T4 DNA polymerase filling reaction and re-ligation. The resulting 3197 base pair plasmid, p434cro2, was used for the construction of combinatorial mutation libraries of the 434 cro gene.

C. Construction of combinatorial libraries of mutations within the DNA encoding the recognition helix in p434cro2:

An oligonucleotide identical in sequence with the DNA between bases 315 and 383 of p434cro2 that included the SacI and BstEII restriction sites of p434cro2 was synthesized with NNS mutagenic codons in several positions of the DNA that encodes the recognition helix of 434 cro. This mutagenic oligonucleotide was annealed to an oligonucleotide primer complementary to its 3′ end and filled in using a T4 DNA polymerase reaction. After restriction with SacI and BstEII, the resultant synthetic double stranded cassette was ligated into SacI and BstEII-cut p434cro2 DNA. The re-ligated combinatorial p434cro2 preparation was electroporated into DH5alpha E. coli. Samples were analyzed at this point to assure that the complete library was represented in the transformed cell preparations. The cells were grown at 370 in LB media without ampicillin for one hour and then ampicillin was added to 50 microgram/ml. The cells were then grown for an additional 8 hours to amplify the plasmid DNA. The plasmid DNA was then isolated by conventional procedures.

D. Screening combinatorial libraries in p434cro2 for targeted DNA binding:

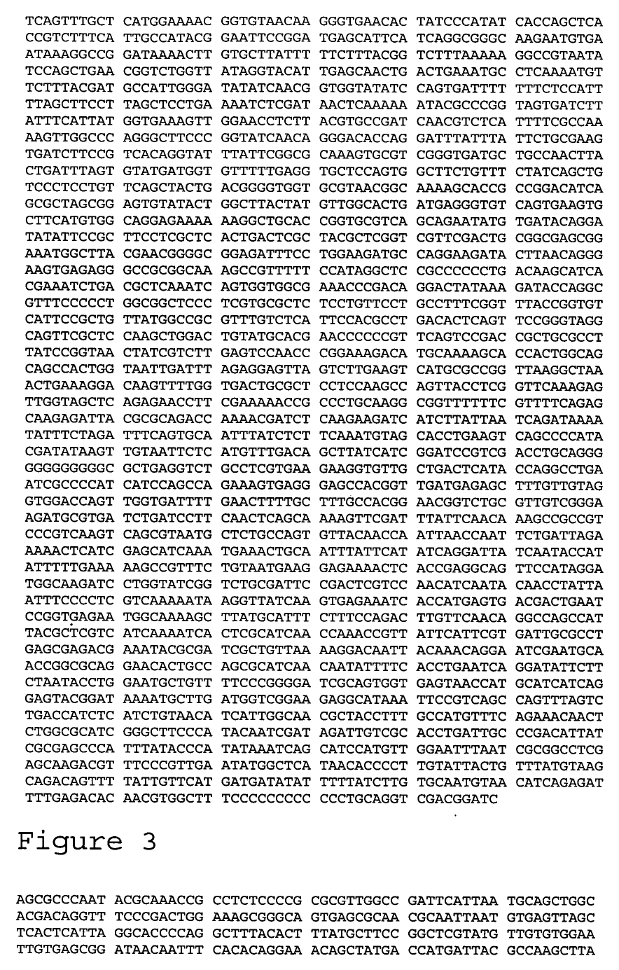

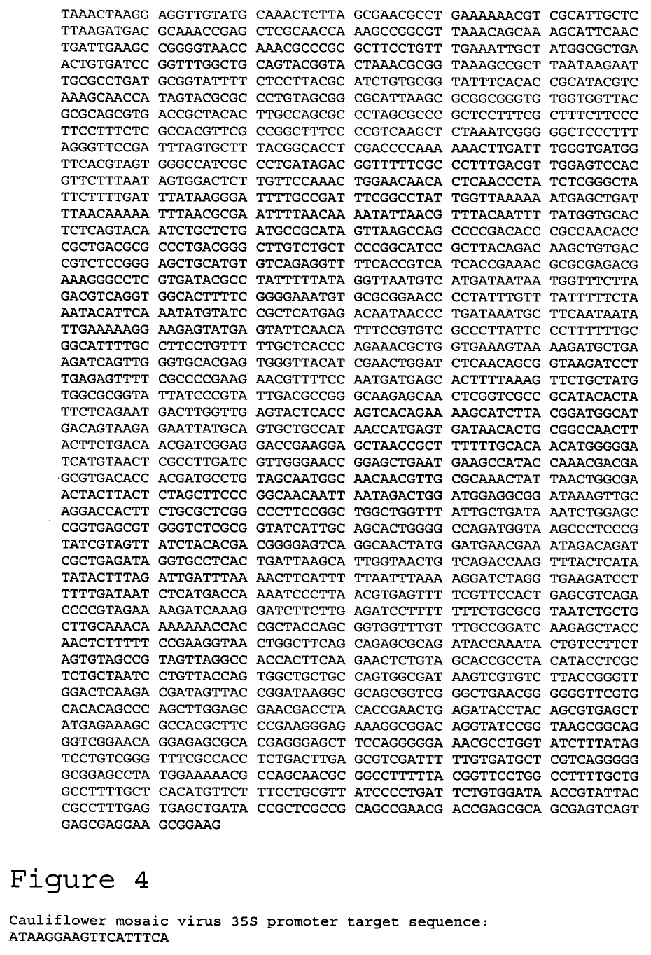

The DNA sequence of pP2HIV1 used in this example is shown in FIG. 2 (SEQ ID NO: 3). The DNA sequence of p434cro2 is shown in FIG. 3 (SEQ ID NO: 4).

E. coli DH5alpha cells containing the pP2HIV1 plasmid were made competent by conventional methods and were subsequently transformed with the 434 cro combinatorial library in p434cro2 DNA. The cells from the transformation were then plated on IM2 media containing and incubated at 370 until colony diameters were between 0.8 and 1.2 mm. The resultant colonies were then optically screened for repression of lacZ′transcription.

The effectiveness of the methods are exemplified by the results using a small combinatorial library. Using a total of only three NNS codons at positions equivalent to Q28, Q29 and S30 of the 434 cro protein, several clones were identified out of the 32,768 possible genetic variations of the 434 cro gene that displayed a repressed lacZ′ phenotype. The p434cro2 variants from these clones were isolated and individually retransformed into cells containing either pP2HIV1 or pP2null plasmids, i.e. screening plasmids identical except for the absence of HIV1 target DNA in the latter, as well as to repressor variants not able to bind DNA (non-repressed controls). Several of the so identified 434 cro variants showed differential repression of the HIV1-derived target DNA. The 434 cro variant with the substitutions Q28C, Q29R and S30A showed the highest level of repression of the target DNA sequence.

### Example 2

### Use of a Separation-Screening Lasmid with ompA-Derived Separator Gene and lacZ-Derived Reporter Gene for the Identification of 434 cro Variants that Bind Cauliflower Mosaic Virus Target Sequences and Construction of DNA Binding Domain-Repressor Domain Fusion Proteins Thereof

A. Description of the Promoter-Target operator-ompA-tag separator/lacZ-reporter plasmid, pComp:

A plasmid, pComp, is constructed from plasmid pP2HIV1, synthetic DNA and DNA derived from the Escherichia coli genome. The plasmid is used to select and screen 434 cro DNA binding protein variants expressed from a repressor plasmid library to DNA target sequences derived from the cauliflower mosaic virus 35S promoter (Rogers, S. G., Klee, H. J., Horsch, R. B. and Fraley, R. T. 1987 Meth. Enz. 153: 253-277).

The first 180 codons of the outer membrane protein ompA in plasmid pComp are isolated from PCR experiments performed with Escherichia coli genomic DNA. This ompA gene fragment encodes the first 159 amino acid residues of the mature ompA protein including its N-terminal signal peptide fused to a synthetic DNA cassette that encodes a streptag peptide sequence. The tagged-ompA fusion protein coding sequence is followed in the plasmid by a lacZ′ derived sequence that encodes an a complementation peptide from the enzyme β-galactosidase. Both the “tagged” ompA fusion protein and the lacZ′ α-peptide are expressed as a polycistronic messenger RNA and are under the transcriptional control of a P2 promoter. A transcriptional terminator sequence synthesized from oligonucleotides based on the transcriptional terminator from the E. coli genome unc operon is inserted into the plasmid after the lacZ′ fragment.

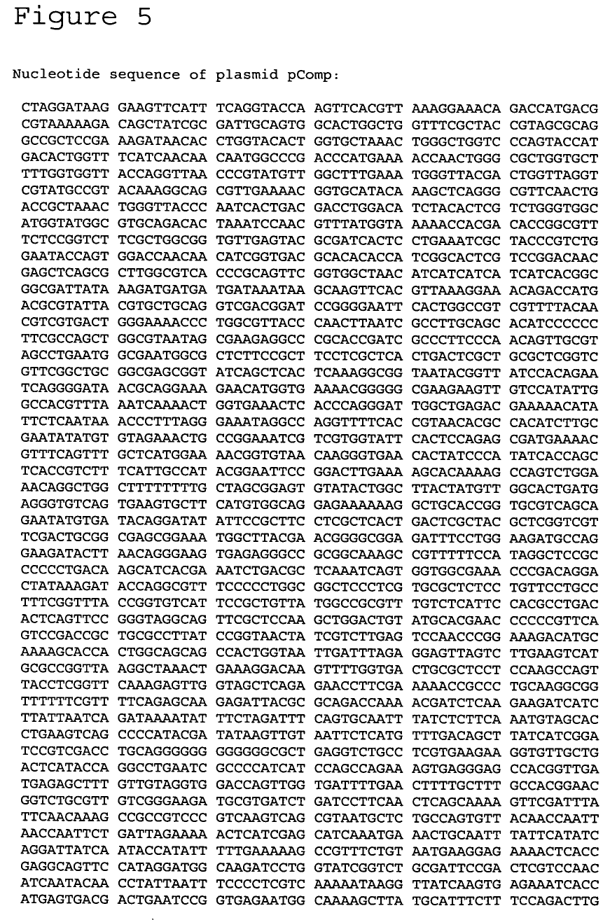

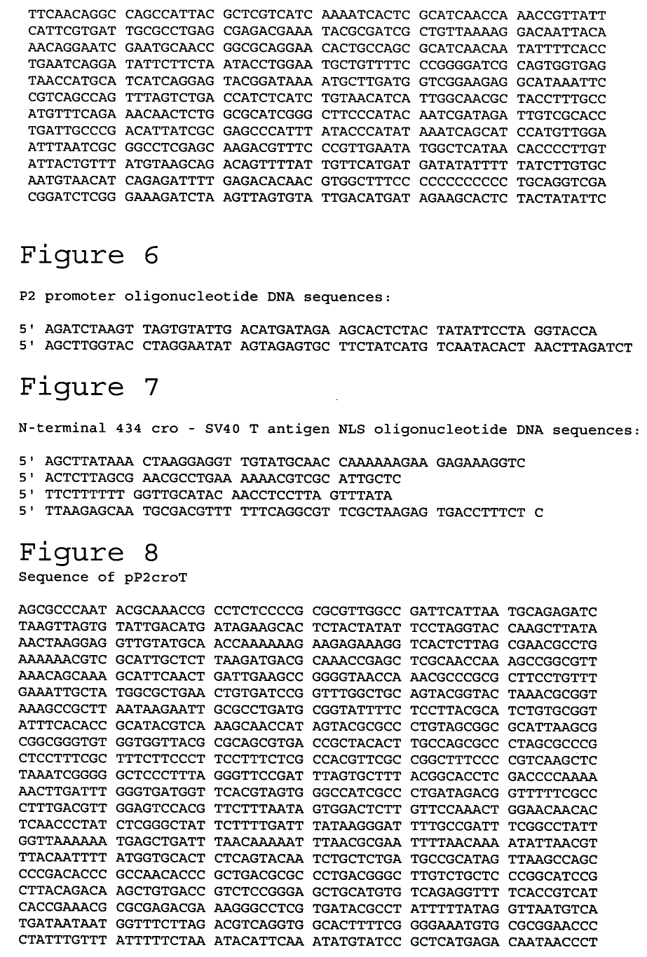

A target DNA sequence derived from the cauliflower mosaic virus 35S promoter (bases 271 to 287 of GenBank file X04879, (Rogers et al ibid.) is positioned between the promoter and ompA-fusion protein in a position where functional operator-repressor interactions are known to occur. The cauliflower mosaic virus 35S promoter target sequence was identified using a computer program that searches DNA sequences for perfect or imperfect palindromic sequences of a definable length. In the case of the operator target sequence used in pComp, two overlapping 14 base pair targets adjacent to the general transcription factor binding site TATA box of the 35S promoter were identified that possessed imperfect palindromic sequences, the outer four bases of which show 75% palindromicity. The DNA sequence of the cauliflower mosaic virus 35S promoter target used in pComp is given in FIG. 4. The DNA sequence of the pComp plasmid is given in the FIG. 5.

Other such targets that are particularly relevant to plant systems and that may bind and compete with other general or specific transcription factors for their binding sites and/or DNA binding sequences that are distinct from those of known transcription factors may alternatively be used. Such specific transcription factor binding sites in plants systems are exemplified but are not limited to the myb family, for example the MYB.PH3 transcriptional activator proteins (Solano, R., Nieto, C., Avila, J., Canas, L., Diaz, I., Paz-Ares, J. 1995 EMBO J. 14:1773-1784), the G-box family, for example the Arabidopsis transcription factor GBF-1 (Schindler, U., Terzagi, W. Beckmann, H. Kadesch, T. Cashmore, AR 1992 EMBO J. 11:1275-1289; Giuliano, G., Pichersky, E., Malik, V. S., Timko, M. P., Sconik, P: A., Cashmore, A. R. 1988 Proc. Nat. Acad. Sci USA 85:7089-7093), the Agamous MADS-box family as exemplified by the proteins Agamous from Arabidopsis thaliana (Huang, H., Mizukami, Y., Hu, Y., Ma, H. 1993 Nucleic Acids Res. 21: 4769-4776; Krizek, B. A., Meyerowitz, E. M. 1996 Proc. Natl. Acad. Sci. USA 93:4063-4070), the O₂ family as exemplified by the Opaque-2 transcriptional activator of maize (Maddaloni, M., Donini, G., Balconi, C., Rizzi, E., Gallusci, P., Forlani, F., Lohmer, S., Thompson, R., Salamani, F., Motto, M. 1996 Mol. Gen. Genet. 250:647-654; Izawa, T., Foster, R., Chua, N.-H. 1993 J. Mol. Biol. 230: 1131-1144), the Athb-1 family (Sessa., G., Morelli, G., Ruberti, 1. 1993 EMBO J. 12:3507-3517; Ruberti, I., Sessa, G., Lucchetti, S., Morelli, G. 1991 EMBO J. 10:1787-1791), the silencer binding factor family as exemplified by the SBF-1 protein from Phaseolus vulgaris (Lawton, M. A., Dean, S. M., Dron, M., Kooter, J. M., Kragh, D. M., Harrison, M. J., Yu, L., Tanguay, L., Dixon, R. A., Lamb, C. J., 1991 Plant Mol. Biol. 16:235-249; Harrison, M. J., Lawton, M. A., Lamb, C. J., Dixon, R. A. 1991 Proc. Natl. Acad. Sci. USA 88:2515-2519) and the myb variant P-box binding family exemplified by the maize activator P protein (Chopra, S., Athma, P., Peterson, T. 1996 Plant Cell 8:1149-1158; Grotewold, E., Drummond, B. J., Bowen, B., Peterson, T. 1994 Cell 76:543-553).

Each of the sequences taught in these references is most particularly contemplated and incorporated by reference, as space limitations preclude recitation of these known sequences.

B. Description of a Combinatorial Library of Repressor Protein Variants Based on the 434 cro Structure, Plasmid pP2croT.

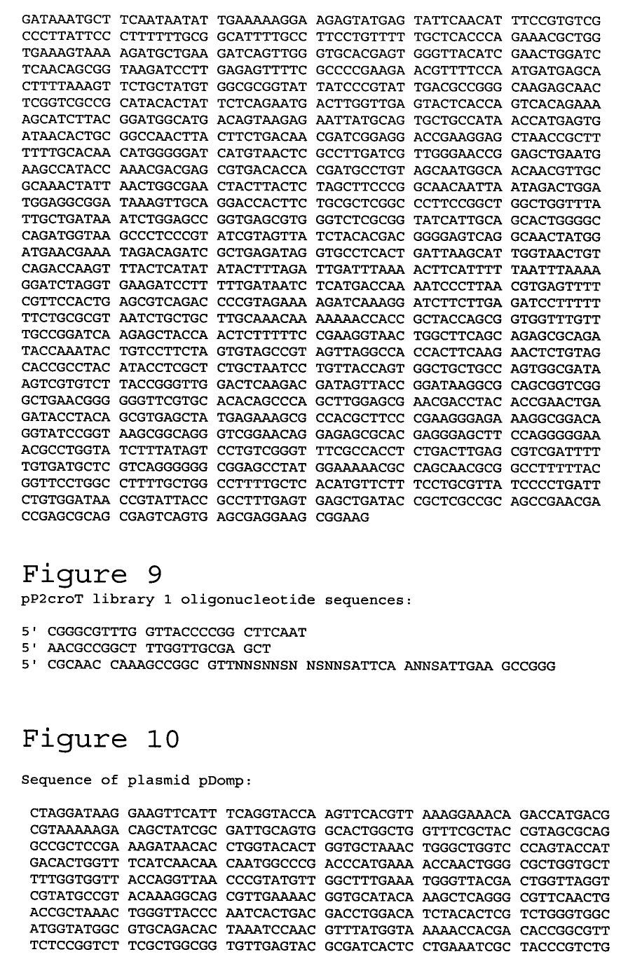

The cro repressor expression plasmid p434cro2 is modified to create pP2croT. This modification lowers the probability of selecting cro repressor variants that repress the expression of the lacZ′ and ompA-fusion proteins of pComp by binding to the P2 promoter instead of the target operator DNA sequence. The modification is carried out by replacing the lacP promoter of p434cro2 that drives the expression of the cro repressor library with the relevant promoter sequence used in pComp. This can be achieved by digesting the p434cro2 plasmid with HindIII and PvuII restriction endonucleases and ligating the resultant vector fragment with a synthetic DNA cassette encoding the P2 promoter that can be assembled from the oligonucleotide sequences shown in FIG. 6. The resultant plasmid is named pP2cro. A DNA sequence different from that used in the pComp plasmid as target operator can be included at the operator position of the pP2croT plasmid to allow counter-selection against possible repressor variants that might bind to an undesired DNA sequence.

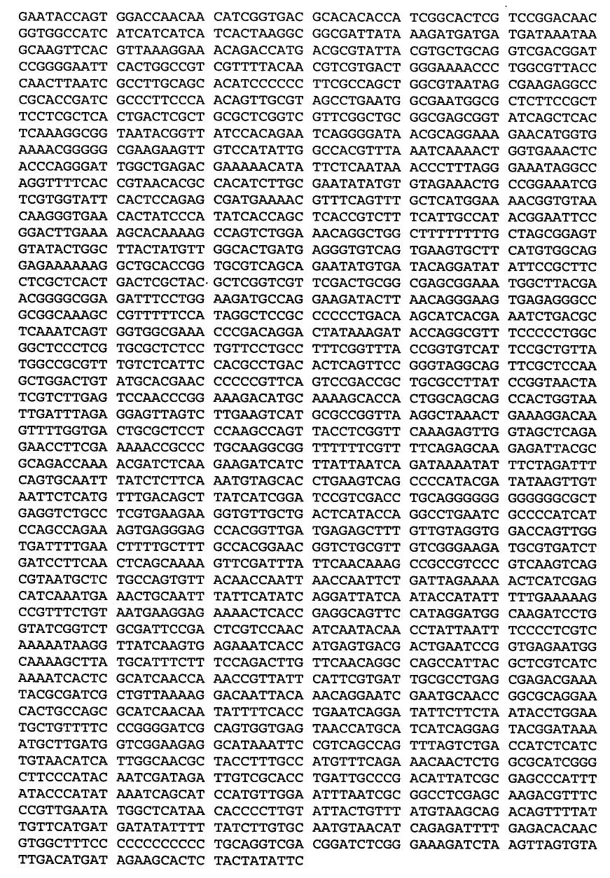

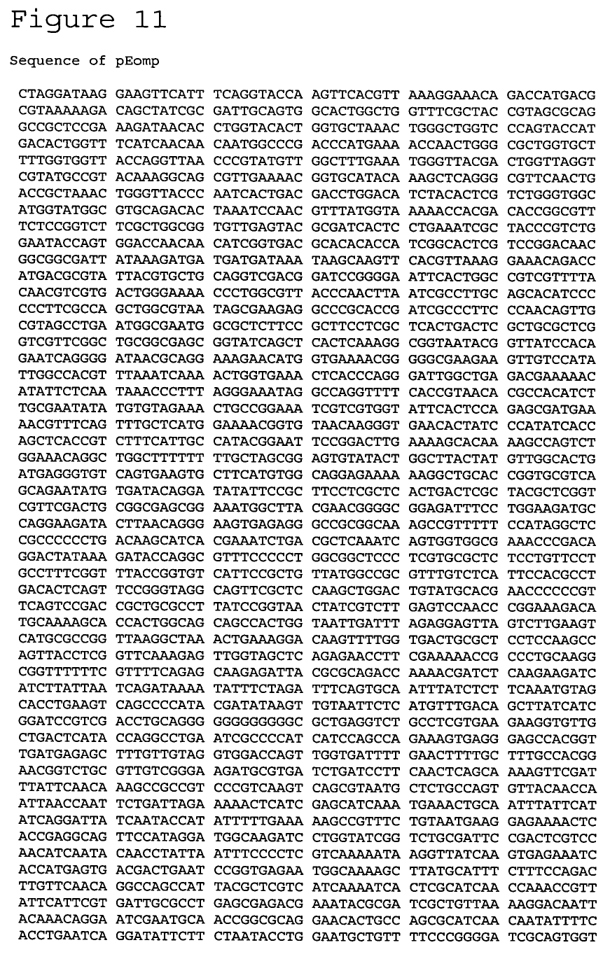

In order to create libraries of cro repressors that possess eukaryotic nuclear localization sequences (NLS) for efficient import into the nuclei of eukaryotic cells, pP2cro can be modified such that the N-terminus of the expressed cro variants are fused with the SV40 T-antigen monopartite NLS having the protein sequence, P K K K R K V. Previous experiments demonstrated that the fusion of large N- or C-terminal protein domains on the 434 cro protein did not significantly influence the DNA binding properties of the cro repressor domain nor did such fusions significantly influence the dimerization of cro monomers nor did it influence functional repression by the dimer. The fusion of the NLS into pP2cro can be achieved by inserting the SV40 T-antigen monopartite NLS into the third codon of the 434 cro gene using a synthetic oligonucleotide cassette encoding DNA between the HindIII and Affll restriction sites of pP2cro that included the SV40 T-antigen encoding sequence. The DNA sequences of these oligonucleotides are given in FIG. 7. The resultant plasmid is named pP2croT. The complete sequence of plasmid pP2croT is shown in FIG. 8.

Combinatorial libraries of mutants of the cro repressor can be constructed in plasmid pP2croT using synthetic oligonucleotides encoding the DNA between the SacI and BstEII restriction endonuclease sites of the plasmid. Except where the amino acid sequence is to be varied, as indicated in the example below, these oligonucleotides preserved the coding sequence of the cro protein variant expressed from pP2croT. An example of a library for use in selecting DNA binding variants of the 434 cro protein varies the amino acids present at the positions corresponding to K27, Q28, Q29, S30 and L33 of the cro protein variant of pP2croT (numbering convention for wild type 434 cro established by Mondragón and Harrison (1991) J. Mol. Biol. 219:321-334 used here). This is accomplished by substituting NNS codons in the oligonucleotides for the unique codons in the respective positions in the cro gene. Such NNS codons are synthesized using approximately equimolar mixtures of the appropriate DNA base precursors in the chemical synthesis of the mutagenic oligonucleotide (NNS, where N=G,A,T or C in the first and second positions of the codons and S=G or C in the third positions of the codons). Other codon combinations as well as mutations at other positions can be made.

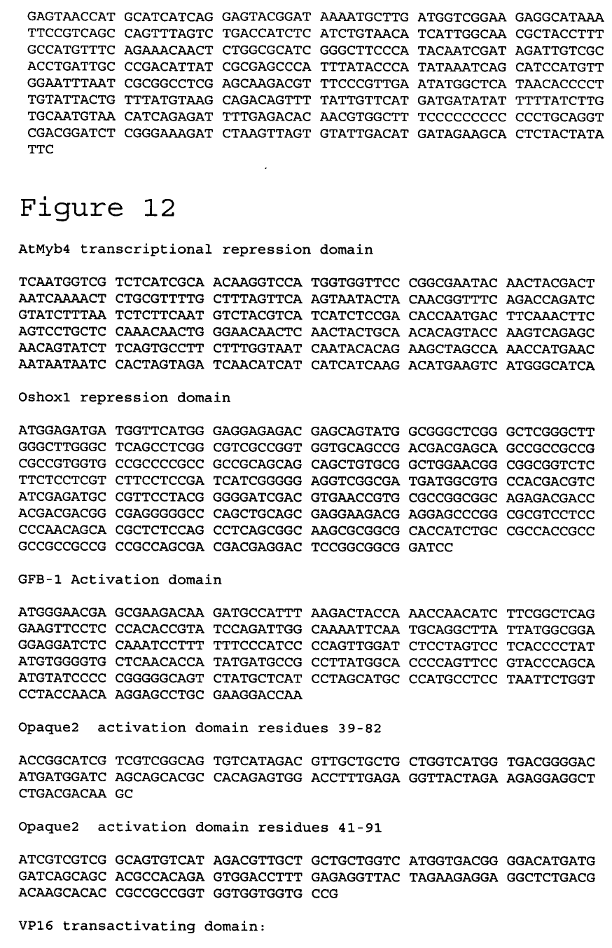

The synthesized mutagenic oligonucleotide can be primed with two oligonucleotides for in vitro DNA synthesis reactions using T4 DNA polymerase and dTTP, dGTP, dCTP and dATP and appropriate buffer solutions. FIG. 9 shows representative sequences of oligonucleotides that can be used. After extraction of the DNA with phenol/chloroform and isopropyl alcohol, the resultant double-stranded DNA cassette can be hydrolyzed with Eco91I, electrophoresed on a 3.8% Metaphor® agarose gel (FMC Corporation), and extracted from the gel using a QIAEX II® gel extraction kit (Qiagen GmbH). This double-stranded cassette then can be ligated into the vector containing fragment of pP2croT obtained from a SacI/Eco91I restriction digestion reaction. The nominal size of the resultant pP2croT library is 3.3554432×10⁷. The preparation should be thoroughly desalted and electroporated into electro-competent DH5α Escherichia coli such that at least >10⁸ transformants are obtained. These cells are then pooled and allowed to grow in liquid LB ampicillin medium for 8 hours at which time the plasmid DNA is isolated from the culture. The purified plasmid DNA is referred to as pP2croT library 1.

C. Selection and Screening of Cro Variants that Bind the Cauliflower Mosaic Virus 35S Promoter DNA Sequences.

Escherichia coli cells from an appropriate strain, for example DH5α, are transformed with plasmid pComp and the cells are allowed to grow to mid-exponential stage in medium containing kanamycin. Before making these cells electro-competent, the cells are treated with an active protease to remove the surface exposed selection tags encoded by the ompA-tagged fusion protein of the plasmid. This is accomplished by adding the protease trypsin to the cell suspension at a final concentration of >4 mg/ml and incubating the cells for a time that can be determined in preliminary experiments that reduces the amounts of surface exposed ompA-tagged fusion proteins to levels that do not interfere with subsequent selection procedures.

The cells treated as described above are transformed by electroporation with enough of the the pP2croT library 1 DNA to produce >10⁸ ampicillin and kanamycin resistant colony forming units. The cells are allowed to recover from the electroporation procedure by the addition of media that includes 0.1 g/l IPTG without antibiotics for 1 hours at 37° and to grow for approximately 1 to 2 generations after addition of ampicillin to 50 Hg/ml and kanamycin to 30 μg/ml.

D. Selection of Repressed and Partially Repressed Cells Containing pComp and cro Variants Expressed from pP2croT Library 1 that Bind the Cauliflower Mosaic Virus 35S Target Sequence Using a Streptag Peptide Separation Technique and lacZ Reporter Gene.

Preliminary to the actual selection experiments a preparation of magnetic particle beads (for example Dynabeads M500 subcellular® from Dynal, A. S., Norway) are coated as described by the manufacturer with a streptavidin protein, for example the variant Strep-Tactin® from IBA GmbH, Germany. Before use, the streptavidin protein variant coated magnetic beads should be washed free of unreacted streptavidin protein by washing at least two times with 2 ml cold buffer containing 100 mM Tris HCl 150 mM NaCl pH 8. After the final wash, the beads should be allowed to settle in a dense slurry. Excess buffer is then removed. Alternatively, StrepTactin-coated magnetic beads can be obtained from IBA GmbH. Multi-well plates are available commercially that are used in an automated variation of this approach to increase the throughput of the method.

The E. coli population containing pP2croT library 1 and the pComp selection plasmid is harvested by centrifugation, washed once and resuspended in 2 ml cold buffer containing 100 mM Tris HCl 150 mM NaCl pH 8. Two hundred μl of a slurry of the streptavidin protein variant coated magnetic beads are added to the cells and allowed to incubate for at least 30 minutes. The tube containing the mixture then is put into the magnetic particle concentrator device (Dynal MPC®-S) and the liquid culture is removed and saved. Six additional, consecutive, step-wise elutions of cells bound to the beads are performed (0.5 ml buffer each containing either 0, 0.1 μM, 1 μM, 10 μM, 100 μM or 2.5 mM D-desthiobiotin). The approximate number of cells in each of the elutions is quantitated by phase-contrast microscopy of an appropriate serial dilution of the respective eluates. Aliquots of the eluates of interest (normally those eluted at lowest D-desthiobiotin concentrations are plated on IM2 indicator medium containing ampicillin and kanamycin such that between 500 and 1000 individual colonies per plate can be observed. These plates are incubated for 18 to 22 hours at 37°. LacZ phenotypes are then observed after 37° incubation and after storage of the plates at 4° for up to 4 hours. At this time colonies that show the desired repressed lacZ-phenotype are picked for further culturing and analysis.

Cro repressor gene containing plasmids are isolated by conventional molecular biological techniques from these cultures. The plasmids are then assayed after individual re-transformation into cells containing reported plasmids possessing either cauliflower mosaic virus 35S promoter DNA target operators, or reporter plasmids having no target operator DNA. Appropriate controls using cro fusion protein variants not able to bind DNA are assayed in parallel with the former samples. Variants of the cro fusion proteins are identified with these techniques that bind to the target DNA sequence from the cauliflower mosaic virus 35S promoter DNA.

In addition to the cro fusion proteins that bind to sequences in the 35S promoter of the cauliflower mosaic virus, additional DNA binding protein variants are selected via binding to different target DNA sequences by individually substituting the DNA sequence given in FIG. 4 in plasmid pComp by other target DNA sequences of interest, for example, from the human genome, HIV and other viral genomes, oncogenic papilloma genomes, other plant and plant-viral promoters, breast and prostate and other oncogene and proto-oncogenes and their promoters as well as others. Application of the techniques given in this example to the new separation-reporter plasmids results in the identification of variants of the DNA binding protein that specifically bind to these additional target DNA sequences.

### Example 3

### Use of a Separation-Screening Plasmid with omPA-Derived Separator Gene and lacZ-Derived Reporter Gene for the Identification of 434 cro Variants that Bind Cauliflower Mosaic Virus Target Sequences Using Alternative Separation-Reporter Plasmids and Alternative Selection Methods

A. Description of the Promoter-Target Operator-ompA-HisTag Separator-Reporter Plasmid, pDomp:

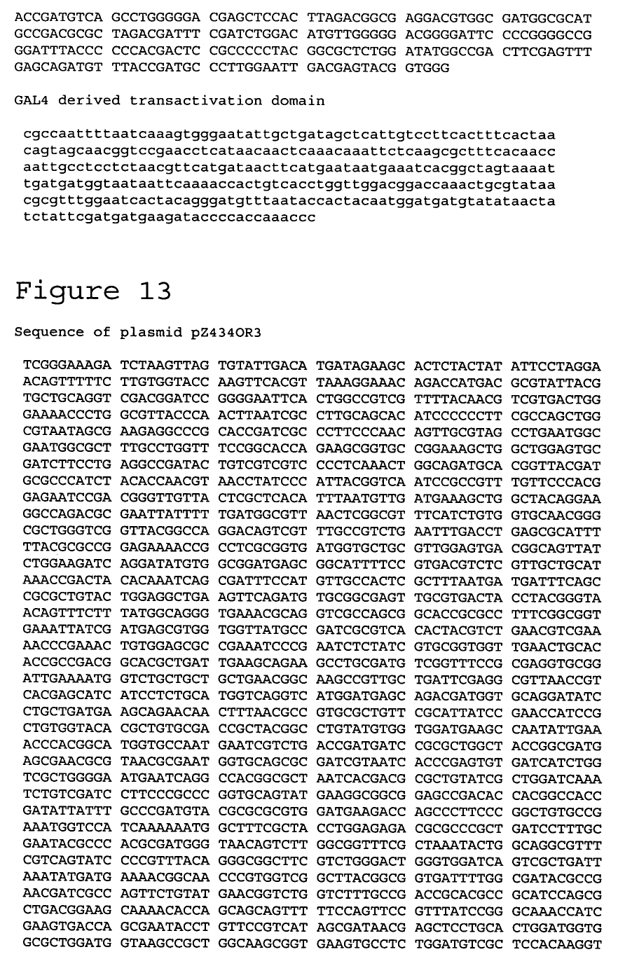

A variation of the selection methods described in Example 2 that can be used to identify DNA binding protein fusions that bind new DNA sequences uses a hexa-histadine protein sequence (“His-Tag”) displayed on the surface of the E. coli cell in place of the Strept-Tag® peptide described for plasmid pComp. FIG. 10 shows the DNA sequence of pDomp. This example of a selection-reporter plasmid employs a HisTag used to select for DNA binding protein variants that bind 35S promoter sequences from the cauliflower mosaic virus. The sequence of plasmid pDomp is identical in sequence to plasmid pComp of example 2 except for the sequence portion that defines the surface displayed tag of the ompA-fusion protein.

B. Selection and Identification of Repressor Protein Variants Using a Promoter-Target Operator-ompA-HisTag Separator-Reporter Plasmid and Ni-Ion Chelation Techniques.

A suitable combinatorial library of mutations of a DNA binding protein domain is constructed as described above for plasmid pP2croT and transformed into cells that contain plasmid pDomp. Cells that contain plasmid pDomp and that are competent for electro-transformation and subsequent selection and screening are prepared as described in Example 2 for cells containing plasmid pComp.

Cells containing variants of the DNA binding protein that encode proteins able to repress the transcription of the genes for the ompA-HisTag separator and reporter proteins of pDomp can be enriched from the total population using either separation methods using Ni-ion chelation or separations using anti-histag specific antibodies.

Enrichment procedures employing Ni-ion chelation techniques begin with the addition of at least 200 μl of a suspension of Ni-NTA magnetic agarose beads (obtained from Qiagen, Inc.) that have been washed as described by the manufacturer and resuspended in 50 mM sodium phosphate containing 30 mM NaCl, pH 8 to the cell suspension containing the DNA binding protein library and pDomp that has been allowed to recover from electro-transformation as described in Example 2. This cell-magnetic bead suspension is incubated at 40 under mild agitation for 1 hour.

At this point the suspension is put into a magnetic particle concentrator. Six individual, consecutive step-wise elutions of cells bound to the beads are performed using 200 μl of a buffer containing 50 mM sodium phosphate containing 30 mM NaCl, pH 8 with 0, 20 mM, 50 mM, 100 mM, 150 mM and 250 mM imidazole.

The approximate number of cells in each of the eluates is estimated as in Example 2. Aliquots of the eluates of interest (normally those eluted at lower concentrations) are then plated on IM2 indicator medium containing ampicillin and kanamycin such that between 500 and 1000 individual colonies per plate can be observed. Plates are allowed to incubate and the identification and isolation procedures as described in Example 2 are followed. Variants of the cro fusion proteins can be identified with these techniques that bind to the target DNA sequence from the cauliflower mosaic virus 35S promoter DNA.

C. Selection and Identification of Repressor Protein Variants Using a Promoter-Target Operator-ompA-HisTag Separator-Reporter Plasmid and Anti-HisTag-Antibody Methods

Alternative enrichment procedures using anti-HisTag antibodies are performed with the same cells containing the pDomp separation-reporter plasmid and the combinatorial library constructed in pP2croT described in B above that have been resuspended in phosphate buffered saline solution containing 0.1% bovine serum albumin.

In this approach, an anti-his-tag antibody coated magnetic bead preparation is first prepared. Mouse IgG monoclonal antibody (for example Penta-His Antibody, Qiagen GmbH) are added to a suspension of Dynabeads Pan Mouse IgG (Dynal, Inc) as described by the bead manufacturer using a ratio of 0.1 to 1 μg IgG per 10⁷ beads. To the cell suspension containing ˜10⁸ cells, between 10⁷ and 10⁹ beads are added and the suspension is gently agitated at 2-8° for 30 minutes. The tube containing the mixture is then put into the magnetic particle concentrator device (Dynal MPC®-S) and the liquid culture is removed and saved. Six additional, consecutive, step-wise elutions of 30 minutes duration of cells bound to the beads are then performed with 0.25 ml of the same buffer each containing either 0, 1, 10, 50, 100, or 250 μg/ml of a synthetic peptide that includes the amino acid residue sequence HHHHHH.

The approximate number of cells in each of the eluates is then estimated as in Example 2. Aliquots of the eluates of interest (normally those eluted at lower hexahistadine peptide concentrations are then plated on IM2 indicator medium containing ampicillin and kanamycin such that between 500 and 1000 individual colonies per plate can be observed. Plates are allowed to incubate followed by identification and isolation as described in Example 2. Using these techniques, variants of the cro fusion proteins are identified that bind target DNA sequence from the cauliflower mosaic virus 35S promoter DNA.

D. Description of the Promoter-Target Operator-ompA-FLAG-Tag Separator-Reporter Plasmid, pEomp:

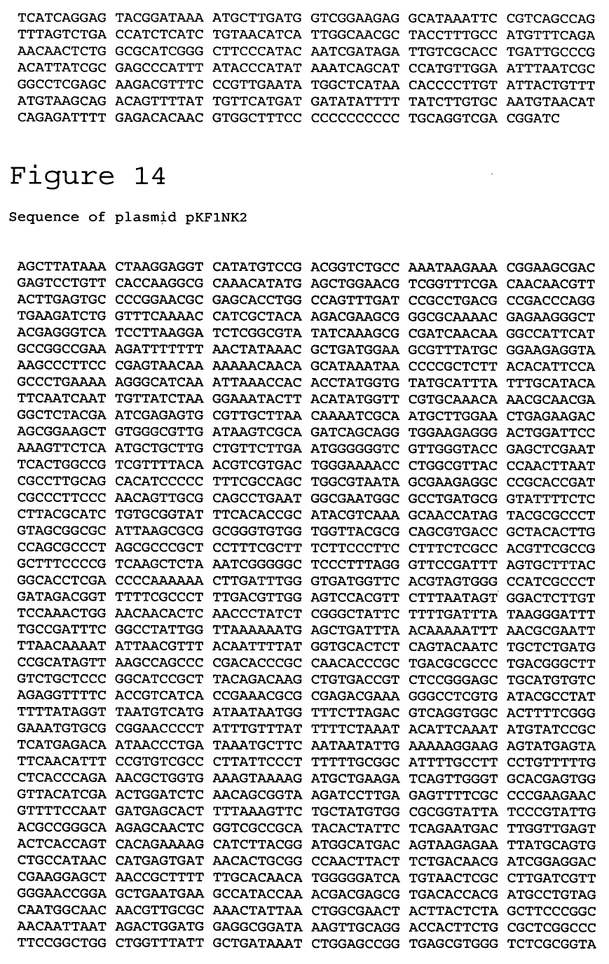

A variation of the selection methods described in example 2 is used to identify DNA binding protein fusions that bind new DNA sequences and uses a FLAG®-Tag protein epitope sequence (protein sequence DYKDDDK, TP Hopp, KS Prickeft, V Price, RT Libby, CJ March, P Cerritti, DL Urdal, PJ Conlon. BioTechnology 6:1205-1210, 1988) displayed on the surface of the E. coli cell in place of the Strept-Tag® peptide described for plasmid pComp. FIG. 12 gives the DNA sequence of pEomp, an example of such a selection-reporter plasmid that employs a FLAG®Tag used to select for DNA binding protein variants that bind 35S promoter sequences from the cauliflower mosaic virus. The sequence of plasmid pEomp is identical in sequence to plasmid pComp of example 2 except for the sequence that defines the surface displayed tag of the ompA-fusion protein.

E. Selection and Identification of Repressor Protein Variants Using a Promoter-Target Operator-ompA-HisTag Separator-Reporter Plasmid and Anti-HisTag-Antibody Methods

A suitable combinatorial library of mutations of a DNA binding protein domain is constructed as described above for plasmid pP2croT and istransformed into cells that containplasmid pEomp. Cells that contain plasmid pEomp and that are competent for electro-transformation and subsequent selection and screening areprepared as in example 2 described for cells containing plasmid, pComp.

Cells containing variants of the DNA binding protein that encode proteins able to repress the transcription of the genes for the ompA-FLAG®Tag separator and reporter proteins of pEomp are enriched from the total population using either separation methods with anti-FLAG®-tag specific antibodies. This is performed with cells containing the pEomp separation-reporter plasmid and the combinatorial library constructed in pP2croT that have been resuspended in phosphate buffered saline solution containing 0.1% bovine serum albumin.

As is similarly described in Example 3C above for the anti-his-tag antibody separation technique, an anti-FLAG®-tag M2 murine antibody coated magnetic bead preparation is first prepared. An M2 anti-FLAG®Tag antibody (available from several suppliers) is added to a suspension of Dynabeads Pan Mouse IgG (Dynal, Inc) as described by the bead manufacturer using a ratio of 0.1 to 1 μg IgG per 10⁷ beads. To the cell suspension containing ˜10⁸ cells, between 10⁷ and 10⁹ beads are added and the suspension is gently agitated at 2-8° for 30 minutes. The tube containing the mixture is then put into the magnetic particle concentrator device (Dynal MPC®-S) and the liquid culture is removed and saved. Six additional, consecutive, step-wise elutions of 30 minutes duration of cells bound to the beads are then performed with 0.25 ml of the same buffer each containing either 0, 1, 10, 50, 100, or 250 μg/ml of a synthetic peptide that includes the amino acid residue sequence DYKDDDDK.

The approximate number of cells in each of the eluates is then estimated as in Example 2. Aliquots of the eluates of interest (normally those eluted at lower peptide concentrations are then plated on IM2 indicator medium containing ampicillin and kanamycin such that between 500 and 1000 individual colonies per plate can be observed. Plates are allowed to incubate and the identification and isolation procedures as described in Example 2 are followed. Variants of the cro fusion proteins can be identified with these techniques that bind to the target DNA sequence from the cauliflower mosaic virus 35S promoter DNA.

F. Alternative Separation Epitope Tag Systems

The examples described here can be expanded with the use of epitope tags of differing sequence to those used in the pComp, pDomp, and pEomp separation-reporter plasmids in combination with appropriate epitope-specific antibodies. Epitope tag examples that can be used are exemplified by but not limited to the HA epitope (protein sequence YPYDVPDYA, H L Niman, R A Houghten, L A Walker, R A Reisfeld, I A Wilson, J M, Hogle, R A Lerner. Proc. Natl. Acad. Sci. USA 80:4949-4953, 1983; I A Wilson, H L Niman, R A Houghten, M L Cherenson, M L Connolly, R A Lerner. Cell 37:767-778, 1984), the c-myc epitope tag (protein sequence EQKLISEEDL, S Munro, HRB Pelham. Cell 48:899-907, 1987), AU1 (protein sequence DTYRYI) and AU5 (protein sequence TDFYLK) epitopes (P S Lim, A B Jenson, C Consert, Y Nakai, L Y Lim, X W Jin, J P Sundberg. J. Infect. Dis. 162:1263-1269, 1990; D J Goldstein, R Toyama, R Dhar, R Schlegel. Virology 190:889-893, 1992), the Glu-Glu epitope (protein sequence EEEEYMPME, T Grussenmeyer, K H Scheidtmann, M A Hutchinson, E Eckhart, G Walter. Proc. Natl. Acad. Sci. USA 82:7952-7054, 1985; B Rubinfeld, S Munemitsu, R Clark, L Conroy, K Watt, W Crosier, F McCormick, P Polakis. Cell 65: 1033-1042,1991), the KT3 epitope (protein sequence PPEPET, H MacArthur, G Walter. J. Virol. 52:483-491, 1984; G A Martin, D Viskochic, G Bollag, P C McCabe, W J Crosier, H Haubruck, L Conroy, R Clark, P O'Connell, R M Cawthon, M A Innis, F McCormick. Cell 63:843-849, 1990), the IRS epitope (protein sequence RYIRS, T C Liang, W Luo, J T Hsieh, S H Lin. Arch. Biochem. Biophys. 329:208-214,1996; W Luo, T C Liang, J M Li, J T Hsieh, S H Lin. Arch. Biochem. Biophys. 329:215-220, 1996), the BTag epitope (protein sequence QYPALT, L F Wang, M Yu, J R White, B T Eaton. BTag: Gene 169:53-58, 1996), the Protein Kinase C epsilon (Pk) epitope (protein sequence KGFSYFGEDLMP, Z Olah, C Lehel, G Jakab, W B Anderson. Anal. Biochem. 221:94-102, 1994) and the Vesicular Stomatitis Virus (VSV) epitope (protein sequence YTDIEMNRLGK, T Kreis. EMBO. J. 5:931-941, 1986, J R Turner, W I Lencer, S Carlson, J L Madara. J. Biol. Chem. 271:7738-7744, 1996).

### Example 4

### Use of Reporter Gene or Separation-Reporter Gene Plasmids and Combinatorial Libraries of DNA Binding Proteins with Fluorescence Activated Cell Sorting (FACS) to Separate Cells Containing Repressor Variants that Bind Desired Target DNA Sequences from Those that do not

A. Use of Reporter Gene Product Substrate Analogs to Identify and Isolate Proteins with new DNA Binding Specificities.

The application of plasmids like pP2HIV1 or pComp in FACS experiments using the β-galactosidase analogs that increase in fluorescence upon hydrolysis by the β-galactosidase enzyme activity can be used to identify members of combinatorial libraries of mutations of DNA binding proteins that bind to desired target DNA sequences. The fact that Escherichia coli can be sorted on the basis of fluorescence intensity has been established for some time (Mia, F., Todd, P., Kompala, D. S. 1993 Biotechnology and Bioengineering 42: 708-715).

Stock solutions of an appropriate substrate analog, for example ImaGene Green C₁₂FDG substrate reagent (Molecular Probes, Inc.) are diluted as described by the manufacturer. Electro-competent cells containing a reporter plasmid as in Example 1 or a separation reporter plasmid as in Example 2 are prepared as described in these examples and combined with the combinatorial library of mutations of the DNA binding protein also as described in examples 1 or 2. After electroporation, cells are allowed to grow at 37° for 90 to 120 in M9 medium containing 0.1 g/L IPTG without antibiotics at which time 30 μg/ml kanamycin and 50 μg/ml ampicillin are added. Growth at 37° is continued for another 60 minutes. Cells are then centrifuged and resuspended in M9 medium with antibiotics that contain 5 μM C₁₂FDC. Staining is allowed to proceed for an additional 90 minutes at 37° in the dark at which point the cell suspension is made 5 mM in phenylethyl-β-D-thiogalactoside. The cells are assayed and sorted on the basis of the fluorescence of the fluorescein moiety using an argon laser at 488 nm in a FACS apparatus. The FACS machine should be set to compensate for the intrinsic auto-fluorescence of the cell culture.

Desired cell fractions, normally those with low fluorescein fluorescence intensities, are then plated on IM2 indicator medium containing ampicillin and kanamycin such that between 500 and 1000 individual colonies per plate can be observed. Plates are allowed to incubate and the identification and isolation procedures as described in Example 2 are followed. Variants of the cro fusion proteins can be identified with these techniques that bind to the target DNA sequence from the cauliflower mosaic virus 35S promoter DNA.

Alternative to the use of reporter gene substrate analogs that become fluorescent upon hydrolysis, fluorescently labeled secondary antibodies can be used in combination with primary antibody labeling of the surface displayed epitope tags. When these methods are similarly applied to FACS experiments, cells containing repressor variants that bind desired target DNA sequences can be separated from those that do not. The repressor variants can then be identified using conventional molecular biology techniques.

### Example 5

### Methods of Creating Fusion Proteins of DNA Binding Domains Identified in Experiments Designed to Identify New Binding Specificities with Domains Capable of Directing Compartmentalization and with Domains Capable of Enhancing Transcriptional Repression Activities or Transcriptional Activation Activities

DNA binding protein fusion protein variants when cloned into appropriate vectors containing appropriate transcription and translation control sequences can compete for binding with endogenous general transcription factors in the cells (for example, TATA binding protein) for the general transcription factor binding sequence, thereby decreasing expression from the targeted promoter. Sequences adjacent to the general transcription factor binding sequence when targeted by the DNA binding protein fusion protein variant can often provide specificity to the variant so that the desired general transcription factor binding site at a specific site in the chromosome can be targeted. In plant cells, for example, fusion protein variants possessing DNA binding domains that bind to sequences from, for example the cauliflower mosaic virus 35S promoter, can be used to decrease gene expression from such promoters. In animal cells, fusion protein variants possessing DNA binding domains that bind to, for example promoter sequences within the HIV1 integrated genome, the HPV genome, or other promoters, can be used to decrease gene expression from the respective promoters. In lower eukaryotes and bacteria similarly, sequence specific DNA binding domains that target general or specific transcription factor binding sites can be used to decrease gene expression from the respective promoters.

Variants of the cro fusion proteins or other DNA binding proteins variants that have been identified that bind, for example, the integrated HIV promoter or the cauliflower mosaic virus 35S promoter, or that bind other targets in other desired promoters that were selected as described above can be further modified by fusion of transcriptional control domains to the C-terminus or N-terminus of the sequence derived from the mutagenized and selected DNA binding domain protein sequence. Such transcriptional control domains that enhance transcriptional repression properties of fusion proteins in plant cells are exemplified by, but not limited to a) the R2R3 Myb gene of Arabidopsis (AtMYB4 gene, amino acid residue numbers 163 to 282, Jin et al. 2000 EMBO J. 19 (22) 6150-61) fused to either the cro N- or C-terminal sequence and (b) sequences derived from the Oshox1 gene from rice (amino acid residues 1 to 155, Meijer et al 2000 Mol. Gen Genet 263; 12-21) fused to either the cro N- or C-terminal sequence. There exist many such transcriptional control domains that are exemplified by, but not limited to the examples given here that can similarly enhance repression of gene activity. In these variations, the repressor activity of the DNA binding domain fusion protein variant can be increased.

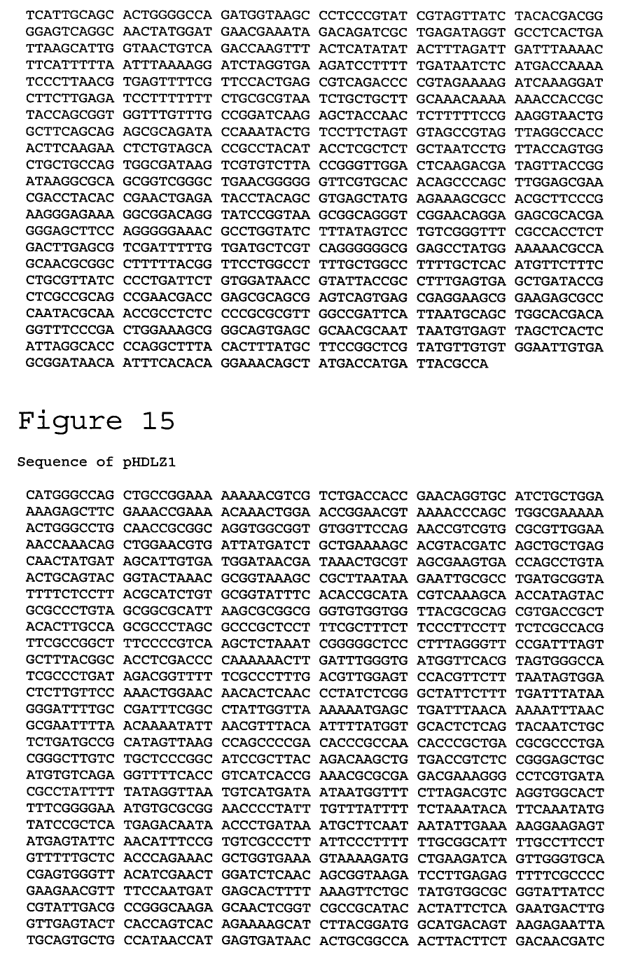

Other variants identified with the plasmids and techniques disclosed here that possess target DNA sequences that are meant to function as new cis-activing activator sequences can be fused with transcription activation domains. In plant cells, such domains as that derived from the N-terminal 110 amino acid residues of the Arabidopsis transcription factor GBF-1 (Schindler, U., Terzagi, W. Beckmann, H. Kadesch, T. Cashmore, AR 1992 EMBO J. 11:1275-1289) can be used. This particular domain has been shown when linked to a DNA binding domain specific for a cis-activating regulatory sequence of a promoter to activate transcription in both plant and mammalian cells. Other domains such as that derived from the N-terminal portion amino acids 39-82 or 41-91 of the Opaque-2 transactivation factor from maize can be fused N-terminally or C-terminally with the DNA binding domain variants that bind the desired target DNA. Additionally, in both animal and plant cells, such domains as the transactivation domain from the VP16 and GAL4 proteins can be used. FIG. 13 gives examples of AtMyb4, Oshox1, GBF-1, Opaque2, GAL4 and VP16-derived transcriptional repression and activation domains that can be fused to DNA binding protein fusions to enhance transcription rates. There exist many examples of transcriptional control proteins that enhance transcription of gene activity that are exemplified by, but not limited to the examples given here that can similarly be used to enhance transcription. Derivatives of these proteins can be fused to DNA binding domains such as derived here to increase transcriptional rates.

In addition to these variations, variants can be created that replace the SV40 T antigen NLS sequences in pP2croT with NLS sequences active in a particular species, for example, the putative nuclear location sequences from Arabidopsis (amino acid residue sequence: KKSRRGPRSR, see for example FIG. 1c of Maes et al 2001 The Plant Cell 13:229-244), or other NLS sequences, for example AAKRVKLG, QAKKKKLDK, PKKKRKV, CNSAAFEDLRVLS and MNKIPIKDLLNPQC (Tung, C.-H. and Stein, S. 2000 Biocongugate Chemistry 11:605-618).

In addition to these variations, examples of fusions with peptide sequences directing cell surface binding, endoplasmatic reticulum retention, cell membrane fusion, lysosomal fusion, membrane translocation plus nuclear localization, RNA binding, artificial nuclease activities as well as other functions such as described by in Tung, C.-H. and Stein, S. 2000 Biocongugate Chemistry 11:605-618 are envisaged.

DNA binding variants can also be constructed also that are intended to influence the transcription of sequences for the import of proteins into subcellular organelles such as mitochondria or chloroplasts, where for example, transcription of organelle specific genes can be influenced.

In addition to these examples, DNA binding variants identified and selected as above can be created that use the sequences of dimeric cro fusion protein variant structures combined into a single chain versions of the corresponding dimeric proteins as exemplified for the 434 repressor (Chen, J. Q., Pongor, S., Simoncsits, A. 1997 Nucleic Acids Res. 25:2047-2054; Simoncsits, A., Chen, J. Q., Peripalle, P., Wang, S, Toro, I., Pongor, S. 1997 Mol. Biol. 267:118-131), λ cro repressor (Jana, R., Hazbun, T. R., Fields, J. D., Mossing, M. C. 2000 Biochemistry 37:6446-6455) and the P22 phage arc repressor (Robinson, C. R., Sauer, R. T. 1996 Biochemistry 35:109-116).

In addition to these examples, heterodimeric and single chain variants can be made that incorporate one monomeric structure of a DNA binding protein variant selected as above with a second variant monomeric structure that binds to a target DNA sequence that is different to that of the first variant. In this way, heterodimeric DNA binding proteins and single-chain variants thereof can be created that possess relatively long, non-palindromic binding sequences made up from the half-sites of the two originally identified homodimers. This method is similar to that taught by (Hollis et al. U.S. Pat. No. 5,554,510) for the creation of heterodimeric DNA binding proteins from monomers having different specificities, but improves on Hollis et al in that many different monomeric structures with differing binding specificities can be produced using the screening and identifications methods given here, as well as by the technique of producing single chain variants thereof.

### Example 6

### Optimization of Promoter Strength in Separation-Reporter Gene Plasmids

Promoter activity of a separator gene/reporter gene polycistron or the promoter activity of a single reporter or separator gene can be optimized to a desired repressor protein by the following methods. It can be observed that strong or weak phenotypes of genes used for separation or reporter activities can mask some combinations of repressor operator transcriptional repression that can be observed when other promoters are utilized. In order not to miss any candidates in selection experiments that use such non-optimized promoter reporter gene combinations, initial optimizations can be performed in a routine manner.

Plasmid pZ434OR3 can be used to illustrate the methods. Plasmid pZ434OR3 (FIG. 13) possesses a nearly full length lacZ reporter gene with a relatively strong lacZ phenotype in comparison to the lacZ′ □ complementation reporter gene used in plasmid pP2HIV1. Plasmid pZ434OR3 also possesses a promoter combined with a target operator equivalent to the OR3 operator of phage 434 (sequence AGATCTMGT TAGTGTATTG ACATGATAGA AGCACTCTAC TATATTCCTA GGAACAGTTT TTCTTGT). The promoter sequence was optimized to the strong lacZ-phenotype by first combinatorially mutagenizing it at several bases within the −10 Pribnow Schaller box and in the −35 consensus sequence (Pribnow, D. 1975 J. Mol. Biol. 99, 419-443; Schaller, H., Gray, C., Herrmann, K. 1975 Proc. Natl. Acad. Sci. USA 72:737-741). This was accomplished using cassette mutagenesis.

The DNA cassettes for the to-be-optimized promoter of pZ434OR3 were constructed with oligonucleotides synthesized with degeneracies at positions within the −35 and −10 consensus sequences. The combinatorial library of promoter mutations can be reconstructed from the mutagenized promoter cassettes and double restricted (BglII and Styl) pZ434OR3. The religated plasmid can then be transformed into an E. coli strain with a lacZ⁻ phenotype and plated on IM2 plates containing kanamycin. Colonies can be picked that show lacZ⁺ phenotypes and plasmids can be prepared from overnight cultures made from these colonies. Plasmids can then be transformed into strains containing a repressor protein known to be able to bind and repress the target operator present in the plasmid. Colonies with optimally repressed lacZ phenotypes can then be isolated, plasmid can be purified, and the sequence of the optimized promoter mutant can be determined by DNA sequencing techniques.

### Example 7

### Determination and Optimization of the Ideal Distance Between the Promoter and Target Operator in the Selection-Reporter Plasmid

An optimal distance between the promoter used to drive transcription of the separation-reporter gene polycistron or single separation or reporter genes can be experimentally determined by the following techniques. A series of separation-reporter gene plasmids can be constructed from the to-be-optimized plasmid, such as pP2HIV1, by restriction of for example the Styl site between the promoter and operator of the plasmid. DNA polymerase fill-in reactions and synthetic cassette and/or linker DNA re-ligations can be performed to generate a series of plasmids that have different DNA sequences and numbers of base-pairs between the promoter and operator sequences. The different distances when unknown can be experimentally determined by DNA sequencing techniques.

When several of these plasmids with different distances between promoter and target operator are tested for observable repression by using separation or reporter phenotypes with a repressor target operator pair that is known to be able to functionally repress transcription, for example the wild-type 434 cro protein and the 434OR3 operator sequence, then an optimal and a maximal distance for observable repression can be determined for that known repressor operator target pair.

This information is valuable for the design and construction of functional separation and/or reporter plasmid target operator sequences that will be applied to the identification of protein variants of DNA binding proteins that bind desired DNA sequences. It is also valuable for the design and construction of functional protein fusions that are desired to be used as pre-mutagenesis structures of DNA binding proteins as is exemplified further below.

### Example 8

### Identification of DNA Binding Proteins that can be Used to Identify Variants that Bind Desired DNA Target Sequences

It can be desirable to use other DNA binding proteins in combination with the separation and reporter techniques exemplified above for the identification of new variants that bind to desired DNA sequences. In this case, the structure of the protein can be optimized so that target DNA sequences can be subsequently identified.

A. Use of homeodomain DNA binding proteins for selection of variants with separation reporter plasmids.

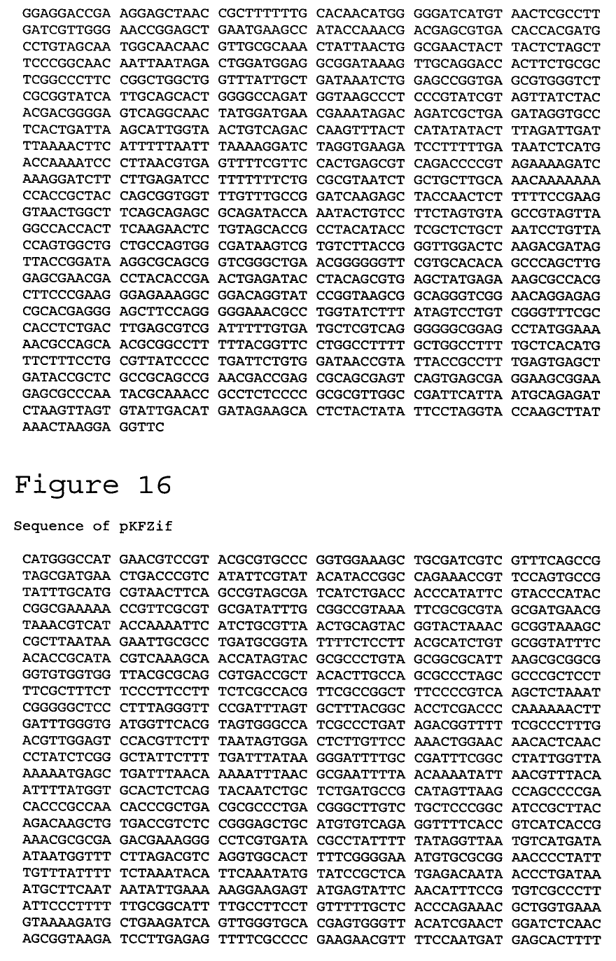

Homeodomain proteins (Gehring, W. J., Affolter, M., Bueglin, T. 1994 Annu. Rev. Biochem. 63:487-526) are large DNA binding proteins involved in transcriptional control and development in eukaryotic cells that contain a relatively small domain (ca. 60 amino acid residues) that binds DNA. These small homeodomains can be expressed as relatively stable proteins and can be used as DNA binding domains that can repress transcription from target operators present in the reporter-separation plasmids described here. An example of such a homeodomain protein can be constructed from the vnd/NK-2 homeodomain proteins first described in Kim, Y. and Niremberg, M. 1989 Proc. Acad. Nath. Sci. USA 86:7716-7720. FIG. 14 gives an example of a plasmid that expresses a vnk/NK-2 homeodomain. When combined in an appropriate E. coli host with a modified reporter separation plasmid (such as pComp) or a modified reporter plasmid (such as pP2HIV1) having NK-2 binding sequences (5′ACTTGAGG) as target operator between the Styl and KpnI restriction sites and optimized as described above, repression of transcription of the separation —reporter polycistronic RNA or the reporter gene RNA can be observed.

Several combinatorial libraries of mutations of the NK-2 homeodomain can be made for example at positions corresponding to R5, K45, 146, Q50, H52, R53, Y54, and/or T56 (numbering as in the consensus homeodomain from Gehring et al, ibid. and Weiler, S. Gruschus, J. M., Tsao, D. H. H., Yu, L., Wang, L.-H., Nirenberg, M., Ferretti, J. A. 1998 J. Biol. Chem. 273:10994-11000) that can be used with separation-reporter gene plasmids or reporter gene plasmids having optimally located target sequences in the methods described above to identify new variants of the NK-2 homeodomain that bind new desired DNA target sequences.

B. Optimization of a Homeodomain-Leucine Zipper Binding Protein for Use with a Separation Reporter Plasmid.

Homeodomain-leucine zipper proteins (HDLZ proteins) are transcription factors that contain both a homeodomain and a leucine zipper dimerization domain (Sessa, G. Morelli, G. Ruberi, 1. 1993 EMBO J. 12:3507-3517) that function most likely in vivo as homodimeric or heterodimeric oligomers. Although so far HDLZ-proteins have only been identified in plants, the small nature of the two domains, their relatively stable independent domain nature and the fact that leucine zipper domains and homeodomains are likely present in every eukaryotic organism, will allow skilled artisans to “mix and match” these two domain types to create new HDLZ proteins from DNA/protein sequences from within any desired species. This will be especially important when new transcriptional control proteins are desired that are not transgenic or that should elicit only minimal immunological responses from a given species.

These HDLZ proteins can also be expressed as relatively stable proteins and can be used as homo- or heterodimeric DNA binding domains that can repress transcription from target operators present in the reporter-separation plasmids described here.

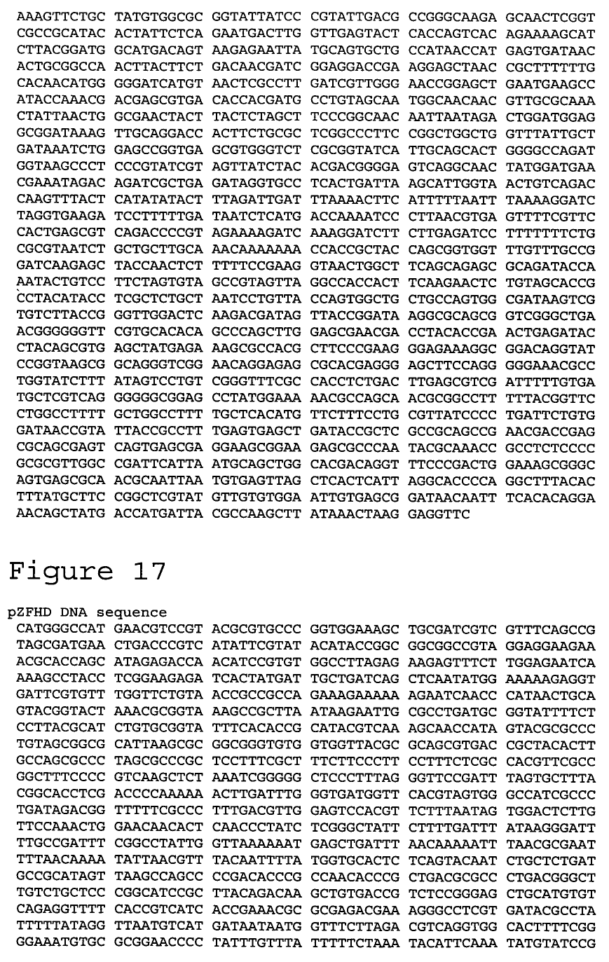

An example of such an HDLZ protein that can be used with the methods presented here can be constructed from for example the ATHB-1 or ATHB-2 proteins described in Sessa, G. Morelli, G. Ruberti, 1. 1993 EMBO J. 12:3507-3517 and Sessa, G. Morelli, G. Ruberti, 1.1997 J. Mol. Biol. 274:303-309. FIG. 15 gives an example of a plasmid, pHDLZ1, that expresses an ATHB-1 HDLZ-fusion protein. When combined in an appropriate E. coli host with a modified reporter separation plasmid (such as pComp) or a modified reporter plasmid (such as pP2HIV1) having an ATHB-1 binding sequences (5′CAAT(A/T)ATTG) as target operator between the Styl and KpnI restriction sites and optimized as described above, repression of transcription of the separation —reporter polycistronic RNA or the reporter gene RNA can be observed.

Several combinatorial libraries of mutations of the ATHB-1 HDLZ-fusion protein can be made for example at positions 45, 46, 50, 52, 53, 54, and/or 56 corresponding to the homeodomain consensus sequence numbering from Gehring et al. (ibid.) that can be used with separation-reporter gene plasmids or reporter gene plasmids having optimally located target sequences in the methods described above to identify new variants of the HDLZ fusion bind new DNA target sequences.

Leucine zipper domain variants of the above HDLZ fusion proteins can be made that preferentially form heterodimeric or homodimeric structures.

C. Optimization of a Zinc-Finger DNA Binding Protein Fusion Binding Protein for Use with a Separation Reporter Plasmid.

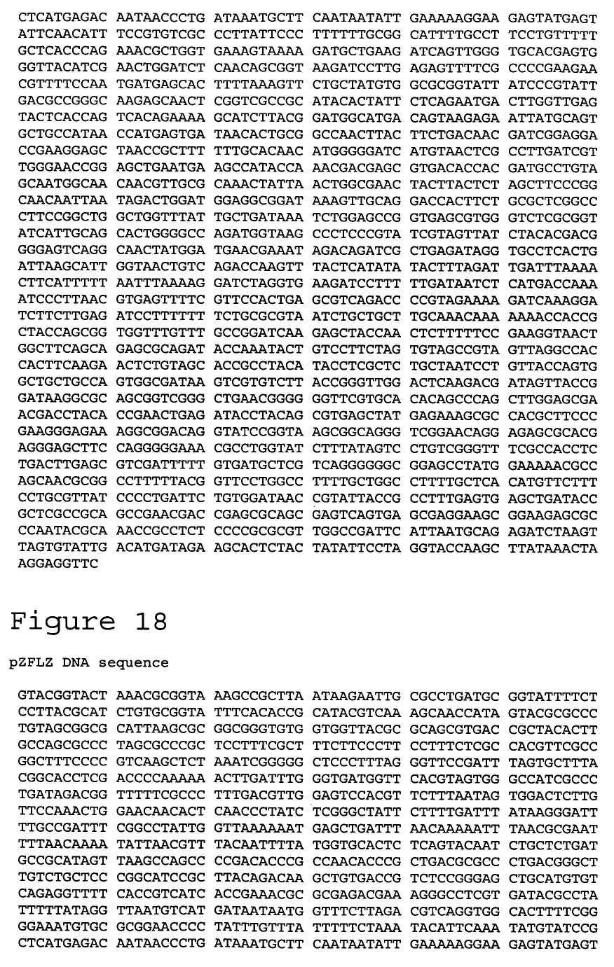

Zinc finger proteins can be used with the methods described here for the identification of new variants that bind altered target DNA sequences. An example of such a zinc finger protein with three individual fingers is the Zif268 immediate early protein (Pavletich, N. P. and Pabo, C. O. 1991 Science 252:809-812). A plasmid, for example pKFZif (FIG. 16), that encodes a truncated Zif268 protein can be used to create and express combinatorial libraries of Zif268 variants that can be used with the methods described here to identify DNA binding specificity variants of a desired sequence specificity. Among the sites to be mutagenized by combinatorial methods are the residues 1, 2, 3, 5 and 6 of the individual zinc finger □-helices as well as the residue −1 that just precedes the zinc finger □-helices.

In order to identify variants that bind desired sequences, a 9 bp target DNA sequence is cloned between the Styl and KpnI sites of separation-reporter plasmid pComp and also reporter plasmid like pP2HIV1. The three finger protein is optimized in three steps, each step being composed of library screens of each individual finger versus a target sequence chimera made from a partially desired sequence and a partial Zif268 consensus binding sequence. These chimeras are constructed such that in the first screen, a library of the first finger is screened versus a target chimera containing three to four bases of the desired sequence combined with six to 7 bases of the Zif268 binding sequence. Consecutive screens of libraries of the remaining fingers versus desired sequences at the appropriate subsites combined with known binding sequences for the remaining fingers yield individual finger variants specific for the desired 9 bp sequence when combined in the appropriate order.

If basal repression levels for individual screens are too high to identify improved binding variants as determined by experiments using specific zinc finger sequences versus chimeric target sequences, then the repression of these target sequences preferably is optimized by including mismatches in the Zif268 binding site sequences. This embodiment reduces the affinity of the protein, lowering its ability to repress the target chimera. Higher affinity binding variants can then be identified that have an increased affinity for the target chimera by virtue of an increased affinity to the desired subsite.

D. Optimization of a Zinc-Finger Homeodomain DNA Binding Protein Fusion for Use with a Separation Reporter Plasmid.

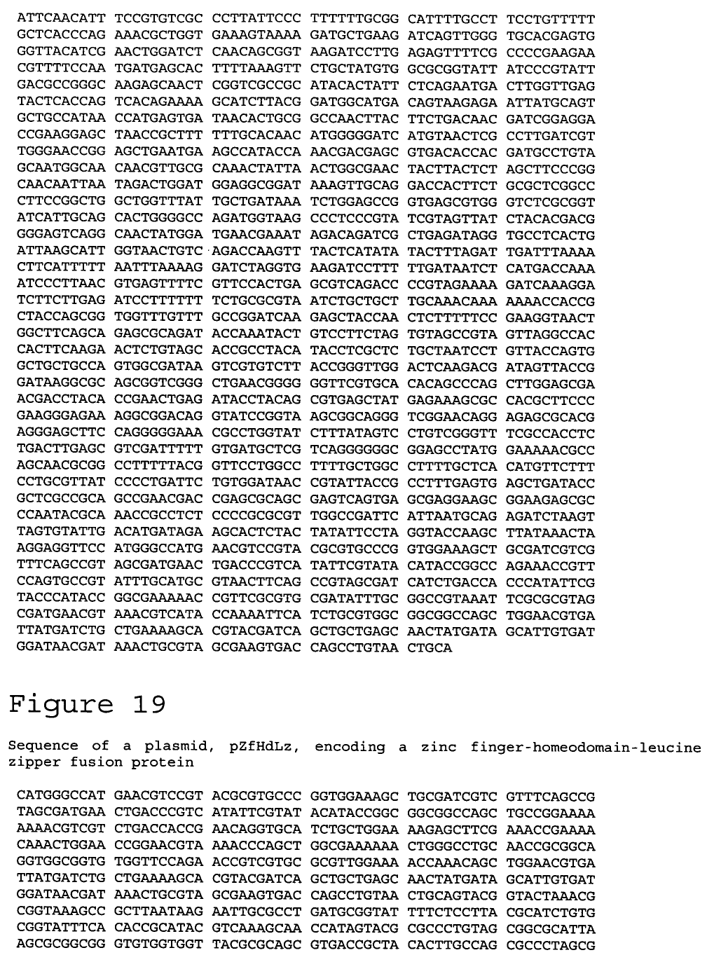

Zinc finger homeodomain fusion proteins described elsewhere are useful in the methods described here for the identification of new variants that bind altered target DNA sequences. An example of such a zinc finger homeodomain fusion protein is that reported by Pomerantz, J. L., Sharp, P. A., Pabo, C. O. 1995 Science 267:93-96. A plasmid, for example PZFHD (FIG. 17), that encodes a similar zinc finger homeodomain fusion protein is used with the methods described above to identify DNA binding specificity variants of a desired sequence specificity. Target DNA sequences that reflect the partial subsites of the high affinity 5′TAATGATGGGCG sequence known for the ZFHD are sequentially identified from libraries of the zinc fingers and homeodomain and combined into the final desired target sequence.

E. Optimization of a Zinc-Finger Dimeric and Zinc-Finger-Homeodomain Dimeric DNA Binding Protein Fusion Binding Protein for use with a Separation Reporter Plasmid.

DNA binding protein fusions can be constructed such that dimerization of monomers will occur. This can be advantageous for certain selections using palindromic and partial palindromic target sequences. Optimization of the distance between half sites can be performed using known partial site binding sequences as described above.

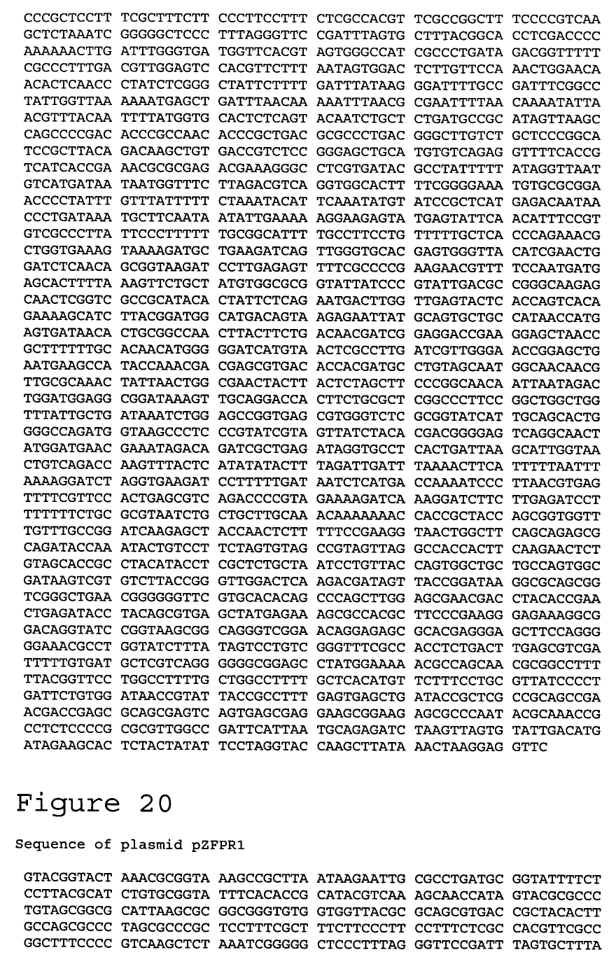

A zinc finger-leucine zipper fusion can be constructed that can be used with the methods described here for the identification of new variants that bind altered target DNA sequences. An example of such a zinc finger-leucine zipper fusion protein is given in FIG. 18.

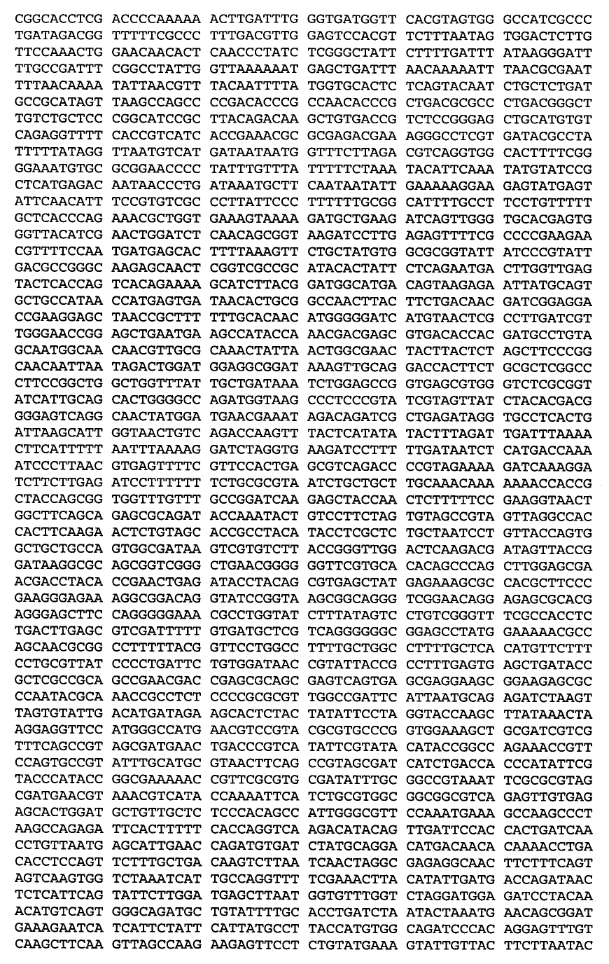

A zinc finger-homeodomain-leucine zipper fusion can similarly be constructed from for example Zif zinc fingers 1 and 2 and the ATHB-1 homeodomain-leucine zipper domains that can be used with the methods described here for the identification of new variants that bind altered target DNA sequences. An example of such a zinc finger-homeodomain-leucine zipper fusion protein is given in FIG. 19.

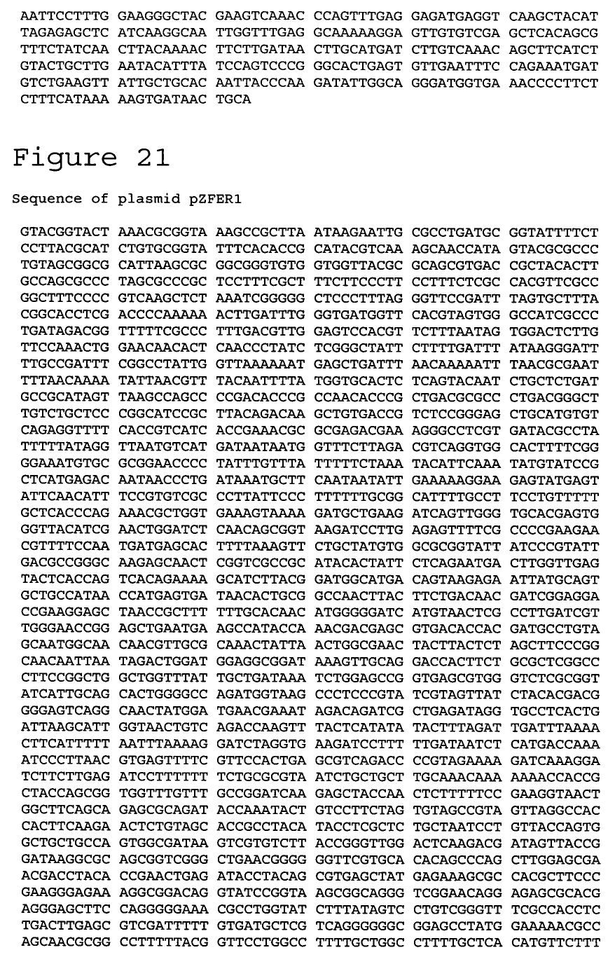

DNA binding domain-hormone dependent dimerization domain fusion proteins like those used by for example Braselmann et al. (1993), Wang et al. (1994) and Beerli et al. (2000) can also be constructed, that when complexed with an appropriate small molecule compound, induce dimerization processes that can lead to DNA binding affinity and specificity increases. A plasmid vector like the pP2croT that encodes a small molecule-dependent dimeric DNA binding protein composed of the a progesterone-dependent dimerization domain fused to a zinc finger DNA binding domain is shown in FIG. 20. This plasmid, pZFPR1, can be used in experiments to identify variants that bind desired DNA target sequences when screening and selection experiments are performed in the presence of an appropriate progesterone analog like RU486. An analog example using a zinc-finger fusion with an estrogen receptor dimerization domain is given in Example 22 (pZFER1). This DNA binding domain estrogen dependent dimerization fusion protein encoding plasmid can be used in the presence of estrogen analogs to similarly identify variants that bind desired DNA target sequences.

Additionally, DNA binding domains can be fused with peptides that direct the dimerization of proteins such as those found in Wang, B. S and Pabo, C. O. 1999 Proc. Natl. Acad. Sci. USA 96: 9568-9573 to create fusion proteins that can be used with the methods described here for the identification of new variants that bind altered target DNA sequences.

### Example 9

Use of DNA binding dependent transcriptional activation and separation-reporter gene expression to identify new DNA binding variants that bind new DNA sequences. The enhancement of transcription of separation tag genes such as those present in plasmids, pComp, that are cloned behind target DNA binding sites recognized by DNA binding protein domain-protein interaction domain fusions can be used to identify new DNA binding variants from combinatorial libraries made from the DNA binding domain. Yeast and bacterial 2- and 1-hybrid systems are known (Chien, C.-T., Bartel, P. L., Sternglanz, R., Fields, S. 1991 Proc. natl. Acad. Sci USA 88: 9578-9582; Wilson, T. E., Padgett, K. A., Johnston, M., Milbrandt, J. 1993 Proc. Natl. Acad. Sci. USA 90:9186-9190; Joung, J. K., Ramm, E. I., Pabo, C. O. 2000 Proc. Natl. Acad. Sci. USA 97: 7382-7387) that use transcriptional activation of reporter genes and genes that complement auxotrophic mutations to identify protein-DNA and protein-protein interactions.

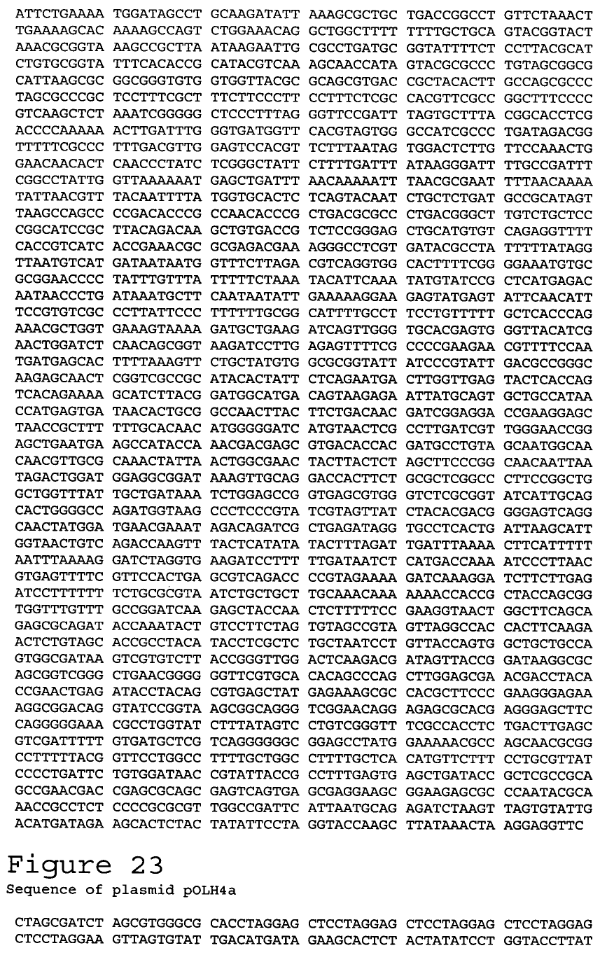

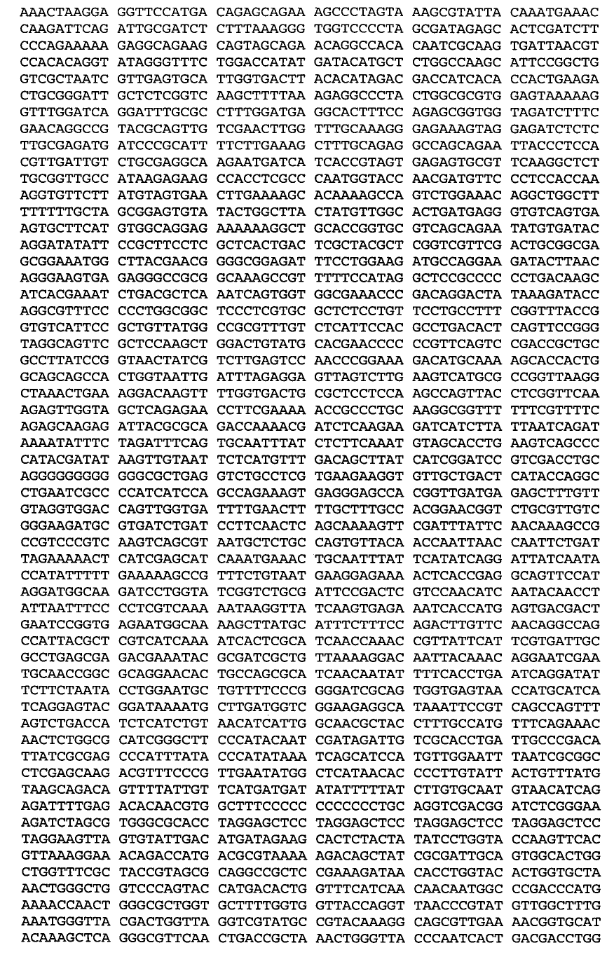

An example of an evolutionarily neutral selection system, i.e. one not utilizing genes that lead to false positives due to unwanted resistance phenomena or undesired transactivation and the like is presented here. This bacterial system uses transcriptional activation instead of transcriptional repression and is exemplified here in the form of methods and plasmids (pPN4, FIG. 22 and pOLH4a, FIG. 23). These constructions are used to directly isolate from combinatorial libraries DNA binding domain variants that bind desired DNA sequences.

Plasmid pPN4 is a derivative of plasmid pKFzif that encodes a yeast GAL11P fused to the Zif268 DNA binding domain and a RNA polymerase alpha subunit (rpoA)-yeast GAL4 protein fusion in a second cistron. Libraries of zinc fingers in pPN4 can be constructed as described in Examples 8C, D and E. A derivative of pComp, pOLH4a can be constructed that has a weak promoter for the separation tag gene and reporter gene (same structural genes as used in Example 2, pComp) and an independent cistron that encodes the yeast HIS3 gene with the same weak bacterial promoter. Transcription of both structural gene sets can be activated by the Zif-RpoA fusion protein encoded on pPN4.

The strength of the weak promoters present and the relative positions in the pOLH4a plasmid can be optimized by the methods in examples 6 and 7. Each of the relevant structural gene sets are isolated in pOHL2 with transcriptional termination sequences and each is bounded on its transcriptionally upstream side with a desired target operator sequence for Zif-RpoA fusion proteins or zinc-finger variants thereof produced as described in Example 8C.

When the pPN4 libraries are combined with plasmid pOLH4a in lacZ-hisB-E. coli cells or when pPN4 libraries that are transformed with lacZ-hisB-E. coli cells that have integrated the pOLH4a sequence into genomic DNA, then cells harboring variants of the Zif268 zinc finger fusion that localize the RNA polymerase-GAL4 fusion to the weak promoter transcription start site of the separation-reporter gene and HIS3 cistrons will grow on histidine-deficient, 3-aminotriazole-containing media. Determination of an optimal 3-aminotriazole concentration in the media to insure HIS3-dependent cell growth is accomplished experimentally in control experiments.

The possibility that cells that grow on histidine-deficient media in these experiments do contain a DNA binding domain that binds target DNA sequences and activates the transcription of the HIS3 gene is deduced by virtue of the expression of the separation tag on the cell surface from the independent cis-acting target sequence. Since this expression is dependent on the same Zif268-GAL11p fusion variant, cells isolated after growth on histidine deficient media by the cell isolation methods presented in Examples 3 through 5 above, eliminate the need for phagemid linkage testing and the problems described by Joung et al 2000 associated with spectinomycin resistance background breakthrough. The procedures exemplified here also eliminate the requirement for negative selection as, for example used by Wilson et al 1993. If in a given experiment, unacceptably high false positives are observed, alternatively, the experiment can be performed in a routine manner using histadine-containing media and the cell isolations methods presented in examples 3 through 5 above.

Variations of these techniques also are contemplated wherein one or more of the separation, reporter or auxotrophic complementation genes and/or RNA polymerase fusion proteins are incorporated in a routine manner into the chromosome of the host.

### Example 10

### Use of Bacteriophage Based Vectors for Separation Tag and Reporter Gene Screening for the Identification of 434 cro Variants that Bind Bacillus Anthracis Derived DNA Sequences

Filamentous bacteriophages have been used for the surface display of combinatorially mutated peptides in procedures known as phage display as described above. These procedures often use one of the closely related f1, fd or M13 filamentous bacteriophages and incorporate mutated peptides or proteins for surface display as fusion proteins in either the gene III or gene VIII coat proteins. Libraries of mutations of variegated fusions are then used in physical separation experiments to identify desired variants.

The use of a viral coat fusion protein of a filamentous virus as a separator gene in analogy with the plasmid-based separation gene-reporter gene system described here can be accomplished by fusing a separation tag (hexahistidinetag, streptag, and/or other tag) with the gene VIII protein coding sequence of a filamentous bacteriophage, for example the M13 bacteriophage. Such a variation for selecting DNA protein variants that bind to new targeted DNA sequences is constructed by the addition of two new operons to the filamentous bacteriophage genome. A promoter, a gene VIII-separation tag fusion protein gene, a reporter gene such as the lacZ′ gene for use in assessing the extent of transcription of the operon and a targeted operator sequence positioned between the promoter and the gene VIII-separation tag fusion in a position where functional operator-repressor interactions are known to occur are combined in a functional unit to create the first operon, the separation-tag reporter gene operon. A second operon is constructed that is used for expression of a DNA binding protein or combinatorial library of mutations of a DNA binding protein that may function as a repressor of the first operon by combining a promoter with appropriate coding and non-coding sequences required for expression of the DNA binding protein. Both of these operons are positioned in the genome of the bacteriophage where they will not interfere with the life cycle of the virus. The filamentous phage vector contains in addition to the gene VIII-separation tag fusion gene a wild type gene VIII in its genome. When the so-constructed virus encodes a desired DNA binding target as operator in the first operon and a library of mutations of a DNA binding protein in the second operon, variants of the DNA binding protein that bind to the targeted DNA binding sequence can be identified via repression of transcription of the gene VIII-separation tag fusion and the lacZ′ reporter gene. A DNA binding protein variant encoded by the second operon in an appropriate bacterial host cell will lower the amount of separation tag present on the surface of the bacteriophage and lower the activity of the reporter gene. Separation of bacteriophages having proportionally more gene VIII separation-tag fusion protein on the surface from those having lower amounts on the virion surface by the use of appropriate separation media, for example hexahistidine nickel ion chelating chromatography gels or solid phase supports, streptavidin or streptag affinity materials or other appropriate separation materials, will enrich the population of bacteriophages having unrepressed from repressed separation tag phenotypes. Plating of the phage fractions proportionally enriched for repressed separation tag on a medium containing Xgal (or other reagent conducive to assaying the reporter gene activity) and the analysis of bacteriophage plaque reporter gene phenotype allows the identification of bacteriophages that encode DNA binding protein variants that bind to the targeted operator sequence.

Use of identical or similar promoters in both operons is advantageous since repressors that bind promoter sequences will self-repress the DNA binding protein and not be identified in subsequence steps. Use of a counter selection operator sequence positioned between the promoter and the DNA binding protein gene in a position where functional operator-repressor interactions are known to occur can result in a lowered probability of identification of any variants that bind the counter-selection sequence since transcription of the DNA binding protein variant that binds the counter-selection operator will be similarly self-repressed. Isolation of the operon containing the gene VIII-separation gene and reporter gene from the operon encoding the DNA binding protein variants with transcription terminator sequences will enhance the efficiency of the system by allowing less run through transcription.

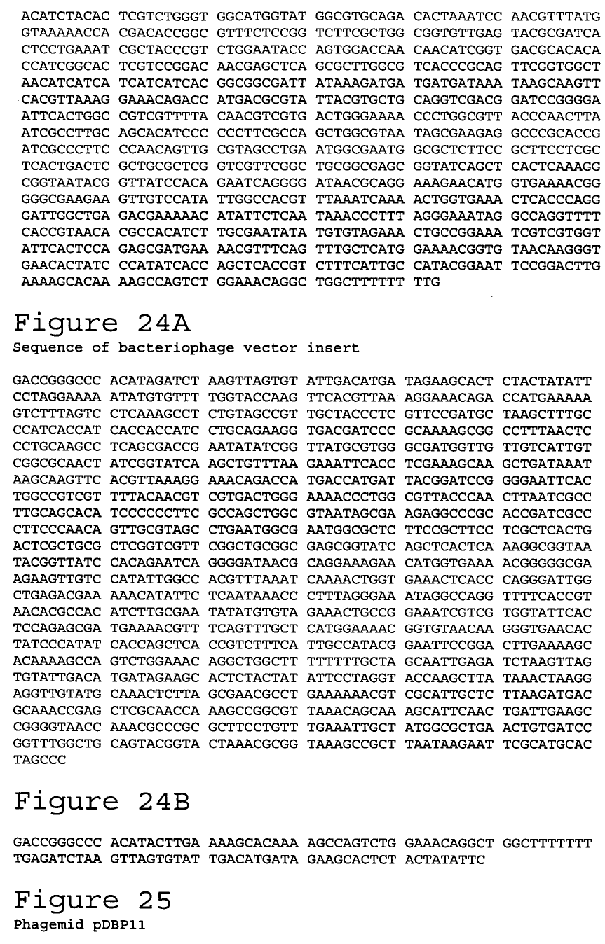

An example of an M13 vector that can be used for the identification of 434 cro variants that bind to Bacillus anthracis target sequences is described here and in FIG. 24. The sequence given in FIG. 24 is part of the genome of an M13 bacteriophage (M2BA1cro1) that contains the gene VIII-separation reporter gene operon and the 434 cro DNA binding protein variant encoding operon and includes unique cloning sites for the construction of bacteriophages containing these operons. The sequence contains a target operon for selection of new DNA binding variants of cro that will bind to the promoter of the atxA promoter of Bacillus anthracis. The functional vector sequence can be obtained by ligating DNA having the sequence in FIG. 24A with the 6655 bp fragment from the M13 mp18 phage cloning vector found between the respective Avall and Bsu361 restriction sites (gene bank entry M77815-basepairs 742 through 5914).

The separator gene used in this vector is the gVIII protein with an hexahistidine fusion tag. The reporter gene is the lacZ′ fragment. The DNA binding protein used is a derivative of the Cro repressor from bacteriophage 434. Mutagenesis of the Cro protein is performed, for example, between the unique SacI and BstEII sites of the vector using cassette mutagenesis and the rationale described for pP2croT above and is ligated into the M2BA1cro1 vector. The combinatorial library is then electroporated into an appropriate E. coli host, for example JM109, and amplified by growth in a rich media. The resultant mixed bacteriophage population is then incubated with hexahistidine nickel ion chelating media and eluted to enrich populations of phage having high amounts of gene VIII fusion protein on the surface from those with low amounts. Populations having increasing amounts of gVIII-hexahistidine tag on the surface can be obtained by elution of phage from the chelation media with increasing concentrations of buffers containing 0, 20, 50, 100, 150, 250 and 500 mM imidazole. Fractions having low amounts of gVIII-Histag on the surface, for example those from elutions having low concentrations of imidazole, are then plated on agar plates containing a inoculate of the appropriate host cells, IPTG and Xgal for assay of reporter gene activity. After incubation at 37° for 14 to 24 hours, plaques should be analyzed for a lacZ phenotype. Those plaques that show the desired repressed lacZ phenotype are then isolated.

Problems with run through transcription where excessive expression of the gene VIII-fusion tag separator and reporter genes occurs through RNA polymerase activities that initiate upsteam of the promoter given in FIG. 24A can be compensated by including a transcriptional terminator sequence between the Avall restriction site of M2BA1cro1 and the minimal promoter of the separation-reporter gene operon. Such a sequence is given in FIG. 24B. For this construction, the sequence of FIG. 24B should replace the first 61 bases of the sequence given in FIG. 24A.

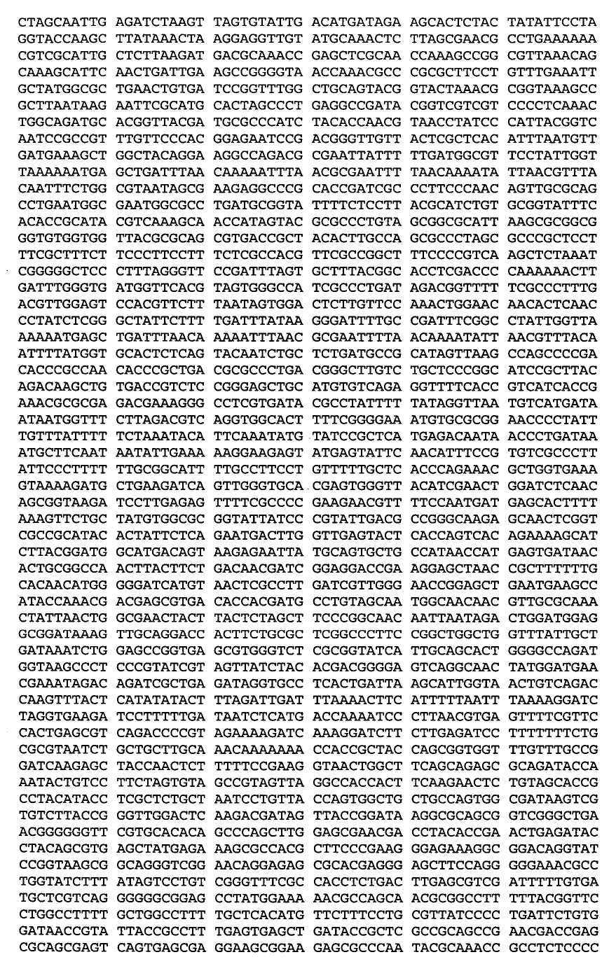

It may also be advantageous to include the operon encoding the DNA binding protein and combinatorial libraries thereof on a phagemid vector like that given in FIG. 25 (pDBP11). When bacteriophage M3BA1 which encodes a separation gene-reporter gene operon like described above for vector M2BA1cro1 but lacks the DNA binding protein operon, new protein variants that bind to the Bacillus anthracis target sequence can be identified from phagemid encoded proteins. M3BA1 is constructed by restriction digestion of M2BA1cro1 with NheI and BstEII followed by T4 DNA polymerase fill-in and ligation. Increases in the ratio of packaged single-stranded phagemid relative to helper phage can be achieved by recloning the 1155 basepair AvaI BstEII fragment isolated from M3BA1 into PstI cut M13KO7 after routine blunting reactions are performed on the fragments. The M13KO7 helper phage is available from Stratagene, USA. Similar strategies of recloning in helper phages R408 or VCSM13 are possible.

It may be desirable to isolate other DNA binding protein variants having other structural binding motifs such as those described above for homeodomain, leucine zipper, zinc finger and combinations of these motifs that target specific DNA sequences. Such protein coding sequences can be incorporated in the M13 or phagemids vectors and used to select mutagenized variants that bind desired target sequences.

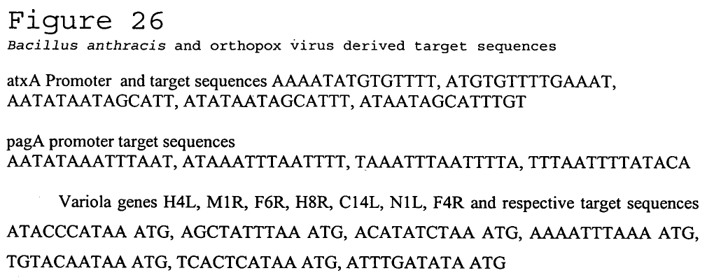

It may be desirable to isolate additional DNA binding proteins that bind Bacillus anthracis derived sequences that may be useful for applications in biodefense or in infectious disease. Such DNA binding protein variants are identified using the methods described above with the target sequences from the atxA and pagA promoters from Bacillus anthracis. Other promoters of interest in biodefense and emerging diseases might be of interest with proteins having such as those derived from the variola H4L, M1R, F6R, H8R, C14L, N1L, F4R genes and their homologs in other orthopox viruses. Examples of such target sequences are given in FIG. 26.

It is also of interest to create bacteriophage vectors that utilize transcriptional activation mechanisms of separator and reporter gene expression to identify new DNA binding protein variants that bind desired DNA sequences.

Linkage of a DNA binding domain to a RNA polymerase localization protein such as a subunit of the polymerase, sigma factor or other protein that locates the RNA polymerase at a desired transcription start region would generate a transactivating regulatory protein. Such a transctivator protein can substitute in the bacteriophage and/or phagemid vectors utilizing the bacteriophage gene VIII fusions described above for the DNA binding repressor protein. Substitution of a low activity promoter in the separator-reporter gene operon of the bacteriophage will result in transcriptional activation of the operon. Use of the bacteriophage coat protein derived separator gene and the cellular reporter gene strategy described above allows the advantageous use of bacteriophage or phagemid encoded libraries of transactivator variants and the genetically neutral screening system.

Such a system can be further modified to create a bacterial two-hybrid system by the physical separation of the coding sequence of a known DNA binding element of the transactivator protein from the RNA polymerase localizing sequences. Fusion of the former domain to a bait protein sequence and fusion of the RNA polymerase localizing sequences to a second “target” protein encoding sequence is performed. Combinatorial mutagenesis of the target sequence fused to the RNA polymerase localizing domain in a phagemid or bacteriophage vector and co-expression with the separator-reporter gene operon can then be used to identify target protein sequences that interact with the bait protein sequence. This is accomplished by separating bacteriophage or phagemid populations that bind to separation materials that interact with the gene VIII-hexahistidine tag bacteriophage coat protein from those that do not bind as readily. Elution of bacteriophage or phagemids from the separation material with buffers containing increasing concentrations of imidazole, for example will yield bacteriophages or phagemids that encode target protein sequences that interact with bait protein sequences to create increasingly better transcriptional transactivators.

Each publication cited is herein incorporated in its entirety by reference. Priority document U.S. No. 60/249,546 entitled “Creation, Identification and use of Proteins with New DNA Binding Specificities” filed Nov. 17, 2000 and PCT/US01/43107 are incorporated by reference in their entirety.

Equivalents

Those skilled in the art will recognize, or be able to ascertain using no more than routine experimentation, many equivalents to the specific embodiments of the invention described specifically herein. These equivalents are included within the scope of the invention.

1. A method for deriving a gene sequence of a DNA binding protein that can bind to a target regulatory sequence, comprising the steps:
a) selecting a starting DNA sequence for a DNA binding protein;
b) mutating the selected sequence of a);
c) providing a mutated DNA sequence from b) to a cell that has at least one genetically neutral transcriptional unit wherein the transcriptional unit comprises at least one promotor, at least one reporter gene or separator gene and at least one copy of the target regulatory sequence, wherein binding between the DNA binding protein encoded by the mutated sequence and the target regulatory sequence regulates the expression of the at least one reporter gene or separator gene; and
d) screening for the regulated expression of a gene from the transcriptional unit.

2. The method of claim 1, wherein the target regulatory sequence of the transcriptional unit is located cis to at least one reporter or separator gene in the transcriptional unit.

3. The method of claim 1, wherein the screening step of d) is carried out with a binding reaction between a probe and a protein that is expressed from a separator gene of the transcriptional unit.

4. The method of claim 1, wherein the screening step of d) is carried out by detection of one or more intracellular components that directly or indirectly form from expression of a reporter gene of the transcriptional unit.

5. The method of claim 1, wherein the transcriptional unit further comprises an operator and wherein the operator is cloned adjacent to a structural gene used for screening and selection, such that binding between the mutated binding protein and the operator sequence regulates expression of the structural gene.

6. The method of claim 1, wherein expression of a reporter in step c) is controlled by binding between the mutated DNA binding protein and the target regulatory sequence.

7. The method of claim 1, wherein expression of a separator gene in step c) is controlled by binding between the mutated DNA binding protein and the target regulatory sequence.

8. The method of claim 1, wherein the DNA binding protein comprises a helix-turn-helix motif structure.

9. The method of claim 1, wherein at least one DNA binding protein is the 434 cro repressor, the NK2 homeodomain or a variant thereof.

10. The method of claim 1, wherein the DNA sequence selection of step a) comprises selecting a DNA sequence that encodes a protein that is known to bind to the target DNA regulatory sequence or to another DNA sequence that has at least a 50% homology to the cognate binding sequence.

11. The method of claim 10, wherein the selected DNA sequence encodes a protein that is known to bind to another DNA sequence that has at least a 70% homology to the cognate binding sequence.

12. The method of claim 11, wherein the selected DNA sequence encodes a protein that is known to bind to another DNA sequence that has at least a 90% homology to the cognate binding sequence.

13. The method of claim 1, wherein the transcriptional unit comprises at least one structural gene encoding a protein selected from the group consisting of lacZ, lacZ′, green fluorescent protein, luciferase, lamB, K88 as pilin, K88ad pilin, TraT, PhoE, OmpA, OmpC, OmpF, OmpF, BtuB, OmpA-lipoprotein fusion, Strep-tag, His-tag, FLAG-Tag epitope, HA epitope, c-myc epitope, AU1 epitope, AU5 epitope, Glu-Glu epitope, KT3 epitope, IRS epitope, BTag epitope, protein kinase C epsilon (Pk) epitope, the Vesicular Stomatitis Virus (VSV) epitope, the M13, fd or f1 gene VIII protein, and the gene III protein.

14. The method of claim 1, wherein at least one reporter gene is lacZ, lacZ′ or a variant thereof.

15. The method of claim 1, wherein at least one reporter gene is β-galactosidase or a variant thereof.

16. The method of claim 1, wherein at least one separator gene encodes a fusion product selected from the group consisting of: ompA, the M13, fd or f1 gene VIII protein; and the M13, fd orf1 gene III protein.

17. The method of claim 1, wherein at least one reporter gene is selected from the group consisting of: OmpA; the M13, fd orf1 gene VIII protein, the M13, and fd orf1 gene III fusion protein, with a peptide selected from the group consisting of: Strept-tag; a hexahistadine-tag; and a hexahistidine flag-tag.

18. The method of claim 1, wherein the cell of step c) is Escherichia coli.

19. The method of claim 1, wherein the target DNA regulatory sequence is selected from the group consisting of: an operator; a regulator sequence of a transcriptional unit containing a reporter gene; and a regulator sequence of a transcriptional unit containing a separator gene.

20. A therapeutic comprising a nucleic acid wherein the nucleic acid comprises a sequence derived by the method of claim 1.

21. A transgenic plant that contains a heterologous gene wherein the heterologous gene comprises a sequence prepared by method comprising the steps:
a) selecting a starting DNA sequence for a DNA binding protein;
b) mutating the selected sequence of a);
c) providing a mutated DNA sequence from b) to a cell that has at least one genetically neutral transcriptional unit wherein the transcriptional unit comprises at least one promotor, at least one reporter gene or separator gene and at least one copy of the target regulatory sequence, wherein binding between the DNA binding protein encoded by the mutated sequence and the target regulatory sequence regulates the expression of the at least one reporter gene or separator gene; and
d) screening for the regulated expression of a gene from the transcriptional unit.

22. A transgenic plant that contains a mutated gene wherein the mutated gene comprises a sequence prepared by method comprising the steps:
a) selecting a starting DNA sequence for a DNA binding protein
b) mutating the selected sequence of a);
c) providing a mutated DNA sequence from b) to a cell that has at least one genetically neutral transcriptional unit wherein the transcriptional unit comprises at least one promotor, at least one reporter gene or separator gene and at least one copy of the target regulatory sequence, wherein binding between the DNA binding protein encoded by the mutated sequence and the target regulatory sequence regulates the expression of the at least one reporter gene or separator gene; and
d) screening for the regulated expression of a gene from the transcriptional unit.

23. A tool for controlling gene expression, comprising a nucleic acid with a sequence obtained by the method of claim 1.

24. A gene having a sequence prepared by the method of claim 1.

25. A vector encoding a gene as described in claim 20.

26. A microorganism that contains a gene as described in claim 24.

27. A library of gene sequences that encode a DNA binding protein, the library prepared by the method of claim 1, wherein the population of cells, bacteriophages or phagemids selected in step d) contains at least 10,000 different DNA binding protein sequences.

28. A library of gene sequences that encode a useful DNA binding protein, the library prepared by the method of claim 1, wherein the population of cells, bacteriophages or phagemids selected in step d) contains at least 1,000,000 different DNA binding protein sequences.

29. A library of gene sequences that encode a useful DNA binding protein, the library prepared by the method of claim 1, wherein the population of cells, bacteriophages or phagemids selected in step d) contains at least 100,000,000 different DNA binding protein sequences.

30. A library of gene sequences of a useful DNA binding protein prepared by the method of claim 1, wherein the population of cells, bacteriophages or phagemids selected in step c) contains at least 10,000,000,000 different DNA binding protein sequences.

31. A method for deriving a gene sequence of a useful DNA binding protein that binds to a target DNA regulatory sequence comprising the steps:
a) selecting a DNA sequence that encodes a protein;
b) mutating the selected sequence of a);
c) providing a mutated DNA sequence from b) to a cell that has at least one genetically neutral transcriptional unit wherein the transcriptional unit comprises at least one promoter and at least one reporter gene or separator gene and at least one copy of the target DNA regulatory sequence, wherein binding between the DNA binding protein encoded by the mutated sequence and the target regulatory sequence regulates the expression of the at least one reporter gene or separator gene; and
d) screening for expression of a gene by the transcriptional unit.
